(19) United States
(12) Patent Application Publication
(10) Pub. No.: US 2023/0132579 A1
(43) Pub. Date: May 4, 2023

(54) PHOTOELECTRIC CONVERSION ELEMENT, IMAGING ELEMENT, OPTICAL SENSOR, AND COMPOUND

(71) Applicant: FUJIFILM Corporation, Tokyo (JP)

(72) Inventors: Yukio TANI, Kanagawa (JP); Hiroki SUGIURA, Kanagawa (JP); Yasunori YONEKUTA, Kanagawa (JP)

(73) Assignee: FUJIFILM Corporation, Tokyo (JP)

(21) Appl. No.: 18/059,397

(22) Filed: Nov. 28, 2022

Related U.S. Application Data
(63) Continuation of application No. PCT/JP2021/020462, filed on May 28, 2021.

(30) Foreign Application Priority Data
May 29, 2020 (JP) 2020-094717
Jul. 30, 2020 (JP) 2020-129186
May 26, 2021 (JP) 2021-088324

(51) Int. Cl. H10K 85/10; H10K 85/20

(52) U.S. Cl. CPC H01L 51/0036; H01L 51/0046; H01L 51/424

(57) ABSTRACT

The present invention is to provide a photoelectric conversion element with an excellent sensitivity, an imaging element, an optical sensor, and a compound. The photoelectric conversion element of the present invention includes, in the following order, a conductive film, a photoelectric conversion film, and a transparent conductive film in which the photoelectric conversion film contains a compound represented by Formula (1) and a coloring agent.

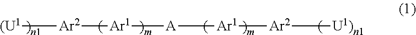

### CROSS-REFERENCE TO RELATED APPLICATIONS

This application is a Continuation of PCT International Application No. PCT/JP2021/020462 filed on May 28, 2021, which claims priority under 35 U.S.C. § 119(a) to Japanese Patent Application No. 2020-094717 filed on May 29, 2020, Japanese Patent Application No. 2020-129186 filed on Jul. 30, 2020 and Japanese Patent Application No. 2021-088324 filed on May 26, 2021. The above applications are hereby expressly incorporated by reference, in its entirety, into the present application.

### BACKGROUND OF THE INVENTION

### 1. Field of the Invention

The present invention relates to a photoelectric conversion element, an imaging element, an optical sensor, and a compound.

### 2. Description of the Related Art

In recent years, the development of an element (for example, an imaging element) having a photoelectric conversion film has been progressing.

For example, a predetermined molecule as an active material of an organic image sensor is disclosed in JP2018-510845A.

### SUMMARY OF THE INVENTION

In recent years, along with the demand for improving the performance of imaging elements, optical sensors, and the like, further improvements are required for various characteristics required for photoelectric conversion elements used therein.

For example, further improvements for sensitivity (photoelectric conversion efficiency) in the photoelectric conversion elements are required.

The present inventors have studied a photoelectric conversion element obtained by using the material disclosed in JP2018-510845A, and have confirmed that there is a room for improving a sensitivity (for example, sensitivity for blue light such as light having a wavelength of 450 nm) in such a photoelectric conversion element.

In view of the above circumstances, an object of the present invention is to provide a photoelectric conversion element with an excellent sensitivity.

Another object of the present invention is to provide an imaging element, an optical sensor, and a compound related to the above-described photoelectric conversion element.

The present inventors have conducted extensive studies on the above-described problems, and as a result, the inventors have found that it is possible to solve the above-described problems by configurations described below and have completed the present invention.

[1]

A photoelectric conversion element comprising, in the following order: a conductive film; a photoelectric conversion film; and a transparent conductive film,

in which the photoelectric conversion film contains a compound represented by Formula (1) and a coloring agent,

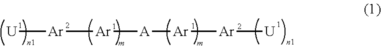

in Formula (1), A is a group represented by any of Formula (2) to Formula (8),

Ar¹ represents an aromatic ring group, where the aromatic ring group represented by Ar¹ may have, as a substituent, a group selected from the group consisting of a halogen atom and an alkyl group having 1 or 2 carbon atoms, which may have a halogen atom,

Ar² represents an aromatic ring group, where the aromatic ring group represented by Ar² may have, as a substituent other than U¹, a group selected from the group consisting of a halogen atom and an alkyl group having 1 or 2 carbon atoms, which may have a halogen atom,

U¹ represents a halogen atom, a cyano group, or an aromatic ring group, where the aromatic ring group represented by U¹ may have, as a substituent, a group selected from the group consisting of a halogen atom, an alkyl group having 1 or 2 carbon atoms, which may have a halogen atom, and a cyano group,

m represents an integer of 0 to 2, and

n1 represents an integer of 0 to 5,

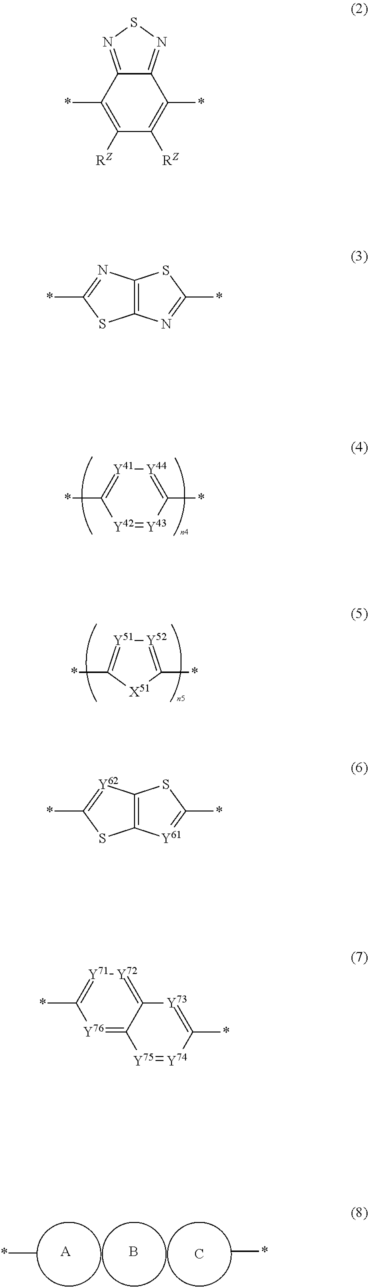

in Formula (2) to Formula (8), * represents a bonding position,

R^{Z} represents a hydrogen atom or a halogen atom,

Y⁴¹ to Y⁴⁴, Y⁵¹, Y⁵², and Y⁷¹ to Y⁷⁶ each independently represent —N═ or —CR═, Y⁶¹ and Y⁶² each independently represent —CR═, R represents a hydrogen atom or a substituent,

X⁵¹ represents a sulfur atom, an oxygen atom, or a selenium atom,

n4 represents 1 or 2,

n5 represents 1 or 2,

A, B, and C each independently represent an aromatic ring group having 5-membered ring or 6-membered ring, A, B, and C are fused with each other to form a fused ring,

in Formula (4), in a case where n4 is 1, at least one of a total of four Y⁴¹ to Y⁴⁴ is —N═ or —CF═, in a case where n4 is 2, at least one of a total of eight Y⁴¹ to Y⁴⁴ is —N═ or —CF═,

in Formula (5), in a case where n5 is 1, at least one of a total of two Y⁵¹ and Y⁵² is —N═ or —CF═, in a case where n5 is 2, at least one of a total of four Y⁵¹ and Y⁵² is —N═ or —CF═,

in Formula (6), at least one of a total of two Y⁶¹ and Y⁶² is —CF═,

in Formula (7), at least one of a total of six Y⁷¹ to Y⁷⁶ is —N═ or —CF═, and

in Formula (8), at least one of ring member atoms constituting the aromatic ring group represented by A, B, and C is —N═ or —CF═.

[2]

The photoelectric conversion element according to [1], in which the compound represented by Formula (1) is a compound represented by any of Formula (1-2) to Formula (1-8),

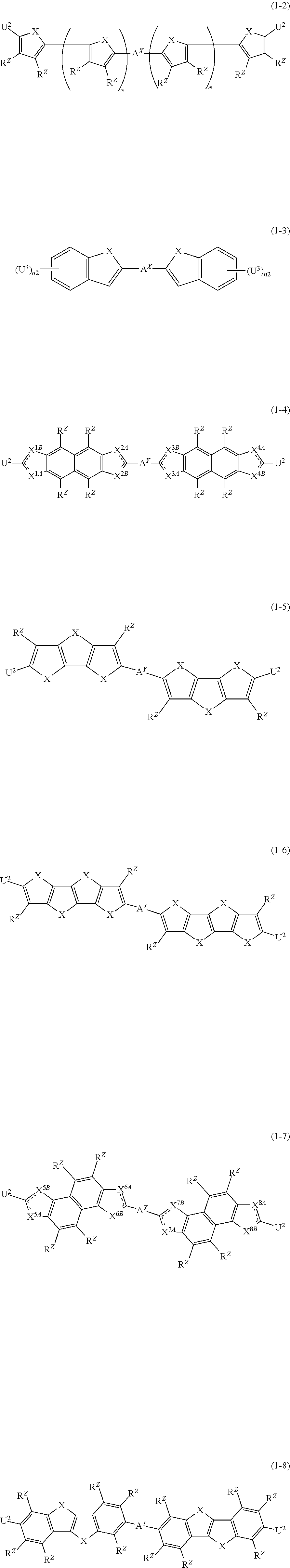

in Formula (1-2) to Formula (1-8), A^{X} is a group represented by Formula (2) or Formula (3),

A^{Y} is a group represented by any of Formula (2) to Formula (8),

U² represents a hydrogen atom, a halogen atom, a cyano group, or an aromatic ring group, where the aromatic ring group represented by U² may have, as a substituent, a group selected from the group consisting of a halogen atom, an alkyl group having 1 or 2 carbon atoms, which may have a halogen atom, and a cyano group,

U³ represents a halogen atom, a cyano group, or an aromatic ring group, where the aromatic ring group represented by U³ may have, as a substituent, a group selected from the group consisting of a halogen atom, an alkyl group having 1 or 2 carbon atoms, which may have a halogen atom, and a cyano group,

R^{Z} represents a hydrogen atom or a halogen atom,

X represents a sulfur atom, an oxygen atom, or a selenium atom,

in Formula (1-4), one of X^{1A} and X^{1B} represents a sulfur atom, an oxygen atom, or a selenium atom, and another of X^{1A} and X^{1B} represents —CR═,

in Formula (1-4), one of X^{2A} and X^{2B} represents a sulfur atom, an oxygen atom, or a selenium atom, and another of X^{2A} and X^{2B} represents —CR═,

in Formula (1-4), one of X^{3A} and X^{3B} represents a sulfur atom, an oxygen atom, or a selenium atom, and another of X^{3A} and X^{3B} represents —CR═,

in Formula (1-4), one of X^{4A} and X^{4B} represents a sulfur atom, an oxygen atom, or a selenium atom, and another of X^{4A} and X^{4B} represents —CR═,

in Formula (1-7), one of X^{5A} and X^{5B} represents a sulfur atom, an oxygen atom, or a selenium atom, and another of X^{5A} and X^{5B} represents —CR═,

in Formula (1-7), one of X^{6A} and X^{6B} represents a sulfur atom, an oxygen atom, or a selenium atom, and another of X^{6A} and X^{6B} represents —CR═,

in Formula (1-7), one of X^{7A} and X^{7B} represents a sulfur atom, an oxygen atom, or a selenium atom, and another of X^{7A} and X^{7B} represents —CR═,

in Formula (1-7), one of X^{8A} and X^{8B} represents a sulfur atom, an oxygen atom, or a selenium atom, and another of X^{8A} and X^{8B} represents —CR═,

R represents a hydrogen atom or a substituent,

m represents an integer of 0 to 2, and

n2 represents 0 or 1.

[3]

The photoelectric conversion element according to [1] or [2],

in which the group represented by Formula (8) is either a group represented by Formula (9) or a group represented by Formula (10),

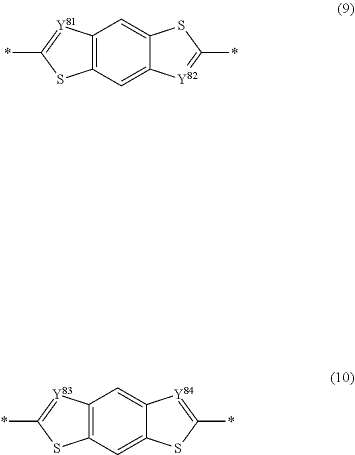

in Formula (9) and Formula (10), * represents a bonding position,

Y⁸¹ to Y⁸⁴ each independently represent —N═ or —CR═, R represents a hydrogen atom or a substituent,

in Formula (9), at least one of a total of two Y⁸¹ and Y⁸² is —N═ or —CF═, and

in Formula (10), at least one of a total of two Y⁸³ and Y⁸⁴ is —N═ or —CF═.

[4]

The photoelectric conversion element according to [1], in which the A is a group represented by any of Formula (3) to Formula (8).

[5]

The photoelectric conversion element according to any one of [1] to [4], in which the compound represented by Formula (1) is a compound represented by any of Formula (1-4) to Formula (1-8).

[6]

The photoelectric conversion element according to any one of [1] to [5], in which the compound represented by Formula (1) has a molecular weight of 400 to 900.

[7]

The photoelectric conversion element according to any one of [1] to [6], in which the photoelectric conversion film is a mixture layer formed in a state where the compound represented by Formula (1) and the coloring agent are mixed.

[8]

The photoelectric conversion element according to any one of [1] to [7], further comprising one or more interlayers between the conductive film and the transparent conductive film, in addition to the photoelectric conversion film.

[9]

The photoelectric conversion element according to any one of [1] to [8], in which the photoelectric conversion film further contains a n-type semiconductor material.

[10]

The photoelectric conversion element according to [9], in which the n-type semiconductor material includes fullerenes selected from the group consisting of a fullerene and a derivative thereof.

[11]

An imaging element comprising the photoelectric conversion element according to any one of [1] to [10].

[12]

An optical sensor comprising the photoelectric conversion element according to any one of [1] to [10].

[13]

A compound represented by Formula (2-2),

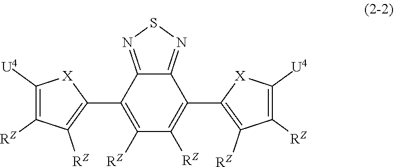

in Formula (2-2), R^{Z} represents a hydrogen atom or a halogen atom,

X represents a sulfur atom, an oxygen atom, or a selenium atom,

U⁴ is a fluorine atom, a group represented by Formula (4-1), or a group represented by Formula (4-2),

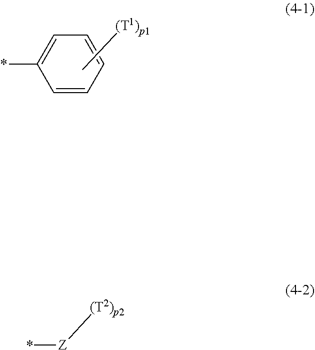

in Formula (4-1) and Formula (4-2), * represents a bonding position,

p1 represents an integer of 1 to 5,

in a case where p1 represents 1, T¹ represents a cyano group,

in a case where p1 represents an integer of 2 to 5, T¹ represents a halogen atom,

Z represents a nitrogen-containing aromatic ring group,

p2 represents an integer of 0 to 4, and

T² represents a halogen atom or a cyano group.

[14]

A compound represented by any of Formula (3-2) to Formula (3-6),

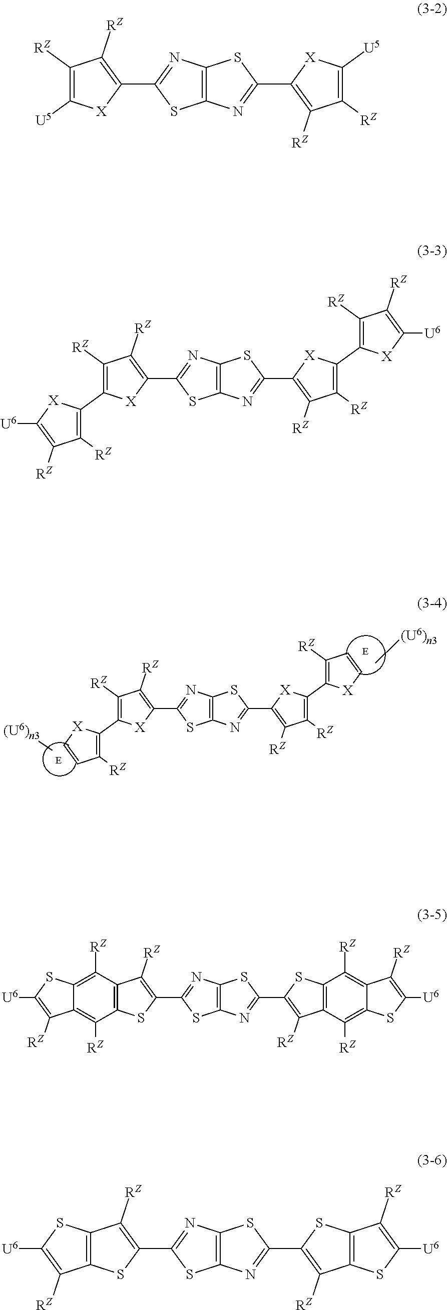

in Formula (3-2) to Formula (3-6), R^{Z} represents a hydrogen atom or a halogen atom,

X represents a sulfur atom, an oxygen atom, or a selenium atom,

E represents an aromatic ring, where the aromatic ring represented by E may have a halogen atom as a substituent other than U⁶,

n3 represents an integer of 0 to 4,

U⁵ is a fluorine atom, a cyano group, a group represented by Formula (4-3), or a group represented by Formula (4-4), and

U⁶ is a fluorine atom, a cyano group, a group represented by Formula (4-5), or a group represented by Formula (4-6),

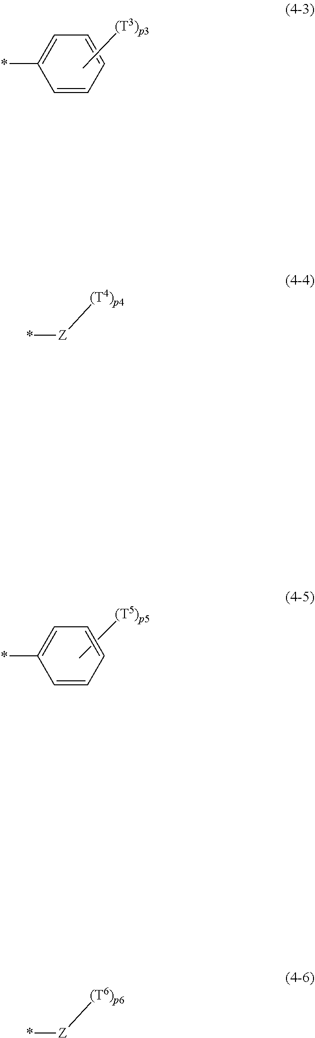

in Formula (4-3) to Formula (4-6), * represents a bonding position,

Z represents a nitrogen-containing aromatic ring group,

p3 represents an integer of 1 to 5,

T³ represents a halogen atom or a cyano group,

p4 represents an integer of 0 to 4,

T⁴ represents a halogen atom, an alkyl group having 1 or 2 carbon atoms, which may have a halogen atom, or a cyano group,

p5 represents an integer of 0 to 5,

T⁵ represents a halogen atom, an alkyl group having 1 or 2 carbon atoms, which may have a halogen atom, or a cyano group,

p6 represents an integer of 0 to 4, and

T⁶ represents a halogen atom, an alkyl group having 1 or 2 carbon atoms, which may have a halogen atom, or a cyano group.

[15]

A compound represented by Formula (1-4),

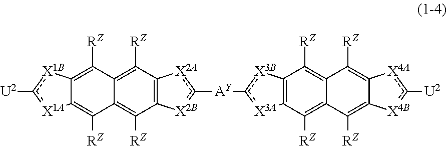

in Formula (1-4), A^{Y} is a group represented by any of Formula (2) to Formula (8),

U² represents a hydrogen atom, a halogen atom, a cyano group, or an aromatic ring group, where the aromatic ring group represented by U² may have, as a substituent, a group selected from the group consisting of a halogen atom, an alkyl group having 1 or 2 carbon atoms, which may have a halogen atom, and a cyano group,

R^{Z} represents a hydrogen atom or a halogen atom,

in Formula (1-4), one of X^{1A} and X^{1B} represents a sulfur atom, an oxygen atom, or a selenium atom, and another of X^{1A} and X^{1B} represents —CR═,

in Formula (1-4), one of X^{2A} and X^{2B} represents a sulfur atom, an oxygen atom, or a selenium atom, and another of X^{2A} and X^{2B} represents —CR═,

in Formula (1-4), one of X^{3A} and X^{3B} represents a sulfur atom, an oxygen atom, or a selenium atom, and another of X^{3A} and X^{3B} represents —CR═,

in Formula (1-4), one of X^{4A} and X^{4B} represents a sulfur atom, an oxygen atom, or a selenium atom, and another of X^{4A} and X^{4B} represents —CR═, and

R represents a hydrogen atom or a substituent,

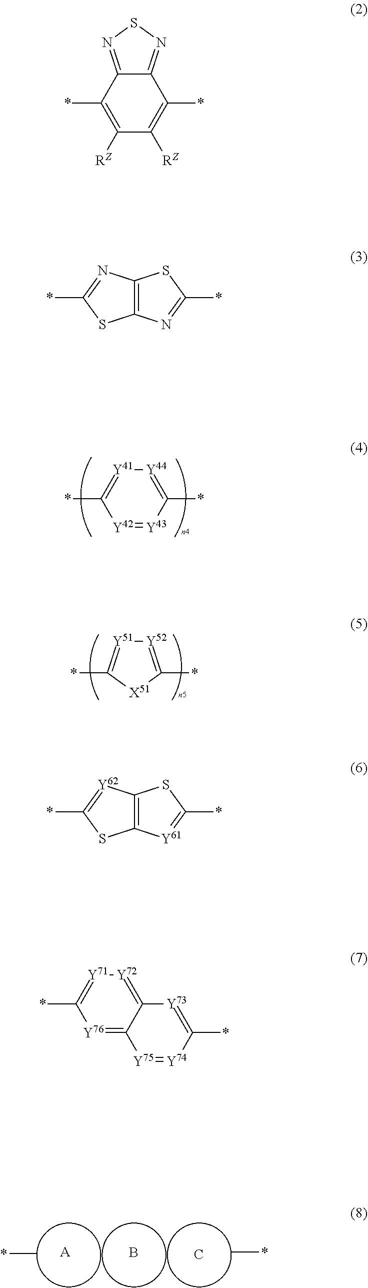

in Formula (2) to Formula (8), * represents a bonding position,

R^{Z} represents a hydrogen atom or a halogen atom,

Y⁴¹ to Y⁴⁴, Y⁵¹, Y⁵², and Y⁷¹ to Y⁷⁶ each independently represent —N═ or —CR═, Y⁶¹ and Y⁶² each independently represent —CR═, R represents a hydrogen atom or a substituent,

X⁵¹ represents a sulfur atom, an oxygen atom, or a selenium atom,

n4 represents 1 or 2,

n5 represents 1 or 2,

A, B, and C each independently represent an aromatic ring group having 5-membered ring or 6-membered ring, A, B, and C are fused with each other to form a fused ring,

in Formula (4), in a case where n4 is 1, at least one of a total of four Y⁴¹ to Y⁴⁴ is —N═ or —CF═, in a case where n4 is 2, at least one of a total of eight Y⁴¹ to Y⁴⁴ is —N═ or —CF═,

in Formula (5), in a case where n5 is 1, at least one of a total of two Y⁵¹ and Y⁵² is —N═ or —CF═, in a case where n5 is 2, at least one of a total of four Y⁵¹ and Y⁵² is —N═ or —CF═,

in Formula (6), at least one of a total of two Y⁶¹ and Y⁶² is —CF═,

in Formula (7), at least one of a total of six Y⁷¹ to Y⁷⁶ is —N═ or —CF═, and

in Formula (8), at least one of ring member atoms constituting the aromatic ring group represented by A, B, and C is —N═ or —CF═.

[16]

A compound represented by Formula (1-5),

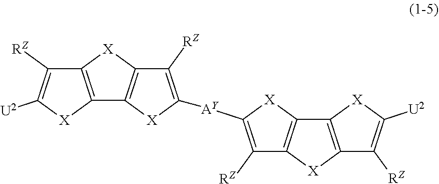

in Formula (1-5), A^{Y} is a group represented by any of Formula (2) to Formula (8),

U² represents a hydrogen atom, a halogen atom, a cyano group, or an aromatic ring group, where the aromatic ring group represented by U² may have, as a substituent, a group selected from the group consisting of a halogen atom, an alkyl group having 1 or 2 carbon atoms, which may have a halogen atom, and a cyano group,

R^{Z} represents a hydrogen atom or a halogen atom, and

X represents a sulfur atom, an oxygen atom, or a selenium atom,

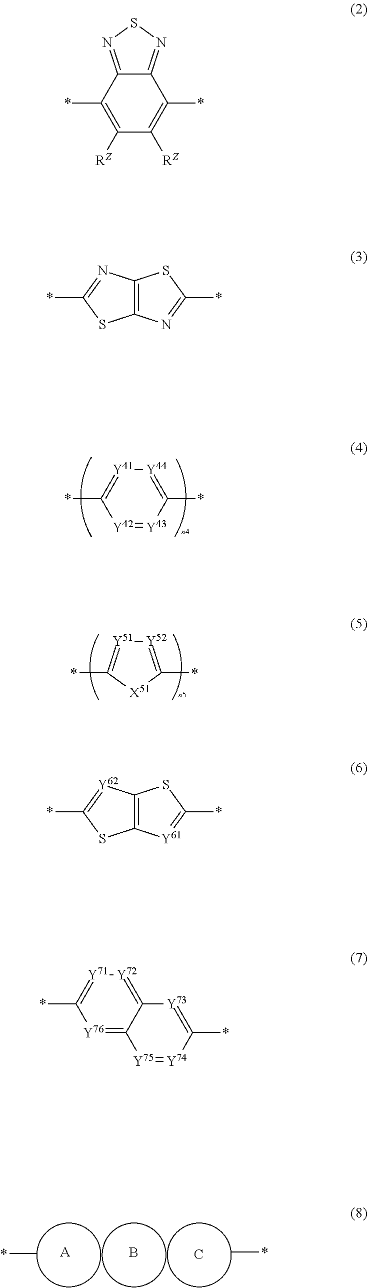

in Formula (2) to Formula (8), * represents a bonding position,

R^{Z} represents a hydrogen atom or a halogen atom,

Y⁴¹ to Y⁴⁴, Y⁵¹, Y⁵², and Y⁷¹ to Y⁷⁶ each independently represent —N═ or —CR═, Y⁶¹ and Y⁶² each independently represent —CR═, R represents a hydrogen atom or a substituent,

X⁵¹ represents a sulfur atom, an oxygen atom, or a selenium atom,

n4 represents 1 or 2,

n5 represents 1 or 2,

A, B, and C each independently represent an aromatic ring group having 5-membered ring or 6-membered ring, A, B, and C are fused with each other to form a fused ring,

in Formula (4), in a case where n4 is 1, at least one of a total of four Y⁴¹ to Y⁴⁴ is —N═ or —CF═, in a case where n4 is 2, at least one of a total of eight Y⁴¹ to Y⁴⁴ is —N═ or —CF═,

in Formula (5), in a case where n5 is 1, at least one of a total of two Y⁵¹ and Y⁵² is —N═ or —CF═, in a case where n5 is 2, at least one of a total of four Y⁵¹ and Y⁵² is —N═ or —CF═,

in Formula (6), at least one of a total of two Y⁶¹ and Y⁶² is —CF═,

in Formula (7), at least one of a total of six Y⁷¹ to Y⁷⁶ is —N═ or —CF═, and

in Formula (8), at least one of ring member atoms constituting the aromatic ring group represented by A, B, and C is —N═ or —CF═.

[17]

A compound represented by Formula (1-6),

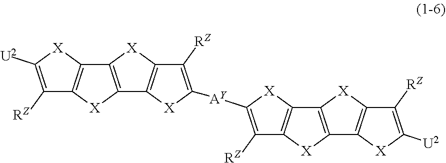

in Formula (1-6), A^{Y} is a group represented by any of Formula (2) to Formula (8),

U² represents a hydrogen atom, a halogen atom, a cyano group, or an aromatic ring group, where the aromatic ring group represented by U² may have, as a substituent, a group selected from the group consisting of a halogen atom, an alkyl group having 1 or 2 carbon atoms, which may have a halogen atom, and a cyano group,

R^{Z} represents a hydrogen atom or a halogen atom, and

X represents a sulfur atom, an oxygen atom, or a selenium atom,

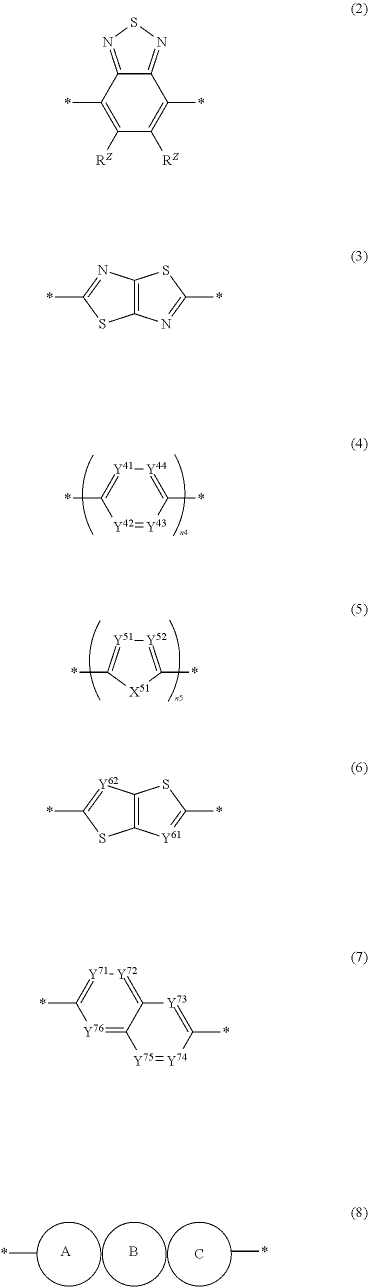

in Formula (2) to Formula (8), * represents a bonding position,

R^{Z} represents a hydrogen atom or a halogen atom,

Y⁴¹ to Y⁴⁴, Y⁵¹, Y⁵², and Y⁷¹ to Y⁷⁶ each independently represent —N═ or —CR═, Y⁶¹ and Y⁶² each independently represent —CR═, R represents a hydrogen atom or a substituent,

X⁵¹ represents a sulfur atom, an oxygen atom, or a selenium atom,

n4 represents 1 or 2,

n5 represents 1 or 2,

A, B, and C each independently represent an aromatic ring group having 5-membered ring or 6-membered ring, A, B, and C are fused with each other to form a fused ring,

in Formula (4), in a case where n4 is 1, at least one of a total of four Y⁴¹ to Y⁴⁴ is —N═ or —CF═, in a case where n4 is 2, at least one of a total of eight Y⁴¹ to Y⁴⁴ is —N═ or —CF═,

in Formula (5), in a case where n5 is 1, at least one of a total of two Y⁵¹ and Y⁵² is —N═ or —CF═, in a case where n5 is 2, at least one of a total of four Y⁵¹ and Y⁵² is —N═ or —CF═,

in Formula (6), at least one of a total of two Y⁶¹ and Y⁶² is —CF═,

in Formula (7), at least one of a total of six Y⁷¹ to Y⁷⁶ is —N═ or —CF═, and

in Formula (8), at least one of ring member atoms constituting the aromatic ring group represented by A, B, and C is —N═ or —CF═.

[18]

A compound represented by Formula (1-7),

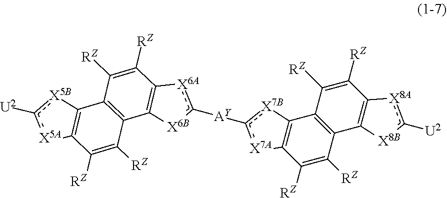

in Formula (1-7), A^{Y} is a group represented by any of Formula (2) to Formula (8),

U² represents a hydrogen atom, a halogen atom, a cyano group, or an aromatic ring group, where the aromatic ring group represented by U² may have, as a substituent, a group selected from the group consisting of a halogen atom, an alkyl group having 1 or 2 carbon atoms, which may have a halogen atom, and a cyano group,

R^{Z} represents a hydrogen atom or a halogen atom,

in Formula (1-7), one of X^{5A} and X^{5B} represents a sulfur atom, an oxygen atom, or a selenium atom, and another of X^{5A} and X^{5B} represents —CR═,

in Formula (1-7), one of X^{6A} and X^{6B} represents a sulfur atom, an oxygen atom, or a selenium atom, and another of X^{6A} and X^{6B} represents —CR═,

in Formula (1-7), one of X^{7A} and X^{7B} represents a sulfur atom, an oxygen atom, or a selenium atom, and another of X^{7A} and X^{7B} represents —CR═,

in Formula (1-7), one of X^{8A} and X^{8B} represents a sulfur atom, an oxygen atom, or a selenium atom, and another of X^{8A} and X^{8B} represents —CR═, and

R represents a hydrogen atom or a substituent,

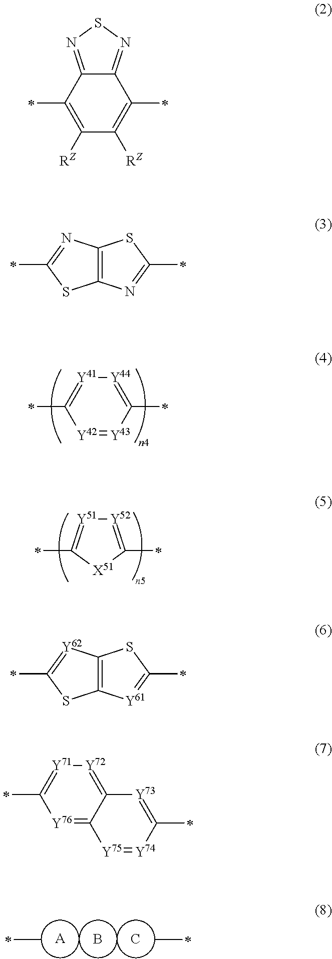

in Formula (2) to Formula (8), * represents a bonding position,

R^{Z} represents a hydrogen atom or a halogen atom,

Y⁴¹ to Y⁴⁴, Y⁵¹, Y⁵², and Y⁷¹ to Y⁷⁶ each independently represent —N═ or —CR═, Y⁶¹ and Y⁶² each independently represent —CR═, R represents a hydrogen atom or a substituent,

X⁵¹ represents a sulfur atom, an oxygen atom, or a selenium atom,

A, B, and C each independently represent an aromatic ring group having 5-membered ring or 6-membered ring, A, B, and C are fused with each other to form a fused ring, n4 represents 1 or 2,

n5 represents 1 or 2,

in Formula (4), in a case where n4 is 1, at least one of a total of four Y⁴¹ to Y⁴⁴ is —N═ or —CF═, in a case where n4 is 2, at least one of a total of eight Y⁴¹ to Y⁴⁴ is —N═ or —CF═,

in Formula (5), in a case where n5 is 1, at least one of a total of two Y⁵¹ and Y⁵² is —N═ or —CF═, in a case where n5 is 2, at least one of a total of four Y⁵¹ and Y⁵² is —N═ or —CF═,

in Formula (6), at least one of a total of two Y⁶¹ and Y⁶² is —CF═,

in Formula (7), at least one of a total of six Y⁷¹ to Y⁷⁶ is —N═ or —CF═, and

in Formula (8), at least one of ring member atoms constituting the aromatic ring group represented by A, B, and C is —N═ or —CF═.

[19]

A compound represented by Formula (1-8),

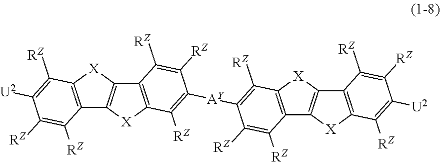

in Formula (1-8), A^{Y} is a group represented by any of Formula (2) to Formula (8),

U² represents a hydrogen atom, a halogen atom, a cyano group, or an aromatic ring group, where the aromatic ring group represented by U² may have, as a substituent, a group selected from the group consisting of a halogen atom, an alkyl group having 1 or 2 carbon atoms, which may have a halogen atom, and a cyano group,

R^{Z} represents a hydrogen atom or a halogen atom, and

X represents a sulfur atom, an oxygen atom, or a selenium atom,

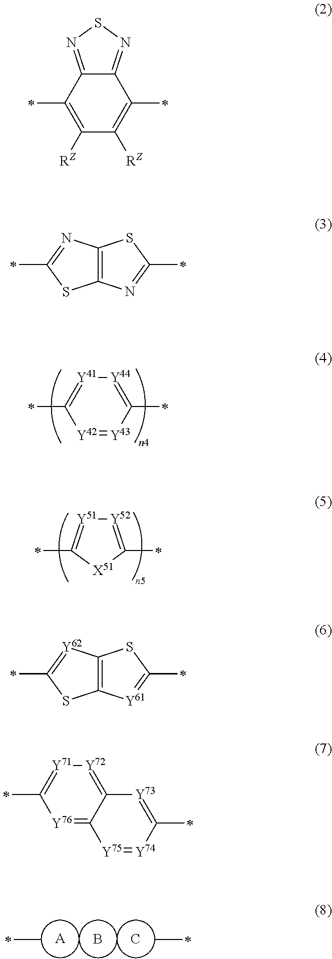

in Formula (2) to Formula (8), * represents a bonding position,

R^{Z} represents a hydrogen atom or a halogen atom,

Y⁴¹ to Y⁴⁴, Y⁵¹, Y⁵², and Y⁷¹ to Y⁷⁶ each independently represent —N═ or —CR═, Y⁶¹ and Y⁶² each independently represent —CR═, R represents a hydrogen atom or a substituent,

X⁵¹ represents a sulfur atom, an oxygen atom, or a selenium atom,

n4 represents 1 or 2,

n5 represents 1 or 2,

A, B, and C each independently represent an aromatic ring group having 5-membered ring or 6-membered ring, A, B, and C are fused with each other to form a fused ring,

in Formula (4), in a case where n4 is 1, at least one of a total of four Y⁴¹ to Y⁴⁴ is —N═ or —CF═, in a case where n4 is 2, at least one of a total of eight Y⁴¹ to Y⁴⁴ is —N═ or —CF═,

in Formula (5), in a case where n5 is 1, at least one of a total of two Y⁵¹ and Y⁵² is —N═ or —CF═, in a case where n5 is 2, at least one of a total of four Y⁵¹ and Y⁵² is —N═ or —CF═,

in Formula (6), at least one of a total of two Y⁶¹ and Y⁶² is —CF═,

in Formula (7), at least one of a total of six Y⁷¹ to Y⁷⁶ is —N═ or —CF═, and

in Formula (8), at least one of ring member atoms constituting the aromatic ring group represented by A, B, and C is —N═ or —CF═.

[20]

The compound according to any one of [13] to [19], in which the compound has a molecular weight of 400 to 900.

According to the present invention, it is possible to provide the photoelectric conversion element with an excellent sensitivity.

In addition, according to the present invention, it is possible to provide the imaging element, the optical sensor, and the compound related to the photoelectric conversion element.

### BRIEF DESCRIPTION OF THE DRAWINGS

FIG. 1 is a schematic cross-sectional view illustrating a configuration example of a photoelectric conversion element.

FIG. 2 is a schematic cross-sectional view illustrating a configuration example of the photoelectric conversion element.

### DESCRIPTION OF THE PREFERRED EMBODIMENTS

Hereinafter, suitable embodiments of a photoelectric conversion element of the present invention will be described.

In the present specification, examples of the halogen atom include a fluorine atom, a chlorine atom, a bromine atom, and an iodine atom.

In the present specification, an aromatic ring group may be monocyclic or polycyclic (for example, with 2 to 6 rings).

The number of ring member atoms of the aromatic ring group is preferably 5 to 15.

The aromatic ring group may be an aromatic hydrocarbon ring group or an aromatic heterocyclic group. The aromatic heterocyclic group may be a nitrogen-containing aromatic ring group described below.

In a case where the aromatic ring group is an aromatic heterocyclic group, the number of heteroatoms included as ring member atoms is, for example, 1 to 10. Examples of the heteroatoms include a nitrogen atom, a sulfur atom, an oxygen atom, a selenium atom, a tellurium atom, a phosphorus atom, a silicon atom, and a boron atom.

Examples of the aromatic hydrocarbon ring group include a benzene ring group, a naphthalene ring group, an anthracene ring group, and a phenanthrene ring group. Examples of the aromatic heterocyclic ring group include a pyridine ring group, a pyrimidine ring group, a pyridazine ring group, a pyrazine ring group, a triazine ring group (1,2,3-triazine ring group, 1,2,4-triazine ring group, 1,3,5-triazine ring group, or the like), and a tetrazine ring group (1,2,4,5-tetrazine ring or the like), a quinoxaline ring group, a pyrrole ring group, a furan ring group, a thiophene ring group, an imidazole ring group, an oxazole ring group, a benzothiophene ring group, a benzothiadiazole ring group, a benzodithiophene ring group (benzo[1,2-b:4,5-b′]dithiophene ring group or the like), a thienothiophene ring group (thieno[3,2-b]thiophene ring group or the like), a thiazolothiazole ring group (thiazolo[5,4-d]thiazol ring group or the like), a naphthodithiophene ring group (naphtho[2,3-b:6,7-b′]dithiophene ring group, a naphtho[2,1-b:6,5-b′]dithiophene ring group, a naphtho[1,2-b:5,6-b′]dithiophene ring group, a 1,8-dithiadicyclopenta[b,g]naphthalene ring group, or the like), a benzothienobenzothiophene ring group, a dithieno[3,2-b:2′,3′-d]thiophene ring group, and a 3,4,7,8-tetrathiadicyclopenta[a,e]pentalene ring group.

In the present specification, in a case where the aromatic ring is simply referred to as an aromatic ring, an example thereof includes an aromatic ring constituting the aromatic ring group.

In the present specification, a nitrogen-containing aromatic ring group is an aromatic ring group having at least one (for example, 1 to 5) nitrogen atom as a ring member atom.

The nitrogen-containing aromatic ring group may be monocyclic or polycyclic (for example, with 2 to 6 rings).

The number of ring member atoms of the nitrogen-containing aromatic ring group is preferably 5 to 15.

The nitrogen-containing aromatic ring group may have a heteroatom other than a nitrogen atom as a ring member atom. The number of heteroatoms other than a nitrogen atom, which are included as ring member atoms, is 0 to 10, for example. Examples of the heteroatoms other than a nitrogen atom include a sulfur atom, an oxygen atom, a selenium atom, a tellurium atom, a phosphorus atom, a silicon atom, and a boron atom.

Examples of the nitrogen-containing aromatic ring group include a pyridine ring group, a pyrimidine ring group, a pyridazine ring group, a pyrazine ring group, a triazine ring group (1,2,3-triazine ring group, 1,2,4-triazine ring group, 1,3,5-triazine ring group, or the like), and a tetrazine ring group (1,2,4,5-tetrazine ring or the like), a quinoxaline ring group, a pyrrole ring group, an imidazole ring group, an oxazole ring group, a benzothiadiazole ring group, and a thiazolothiazole ring group (thiazolo[5,4-d]thiazol ring group or the like).

Examples of the alkyl group having 1 or 2 carbon atoms, which may have a halogen atom, include an unsubstituted methyl group, an unsubstituted ethyl group, a methyl group having 1 to 3 halogen atoms, and an ethyl group having 1 to 5 halogen atoms.

The alkyl group having 1 or 2 carbon atoms, which may have a halogen atom, may be a trifluoromethyl group, for example.

In the present specification, in a case where a plurality of identical symbols indicating a kind or the number of groups are present in Formula (General Formula), which indicates a chemical structure, contents of these plurality of identical symbols indicating a kind or the number of groups are independent of each other, and the contents of the identical symbols may be the same or different from each other unless otherwise specified.

In addition, in the present specification, the numerical range represented by “to” means a range including numerical values denoted before and after “to” as a lower limit value and an upper limit value.

In the present specification, a hydrogen atom may be a light hydrogen atom (an ordinary hydrogen atom) or a deuterium atom (a double hydrogen atom and the like).

[Photoelectric Conversion Element]

The photoelectric conversion element according to an embodiment of the present invention includes a conductive film, a photoelectric conversion film, and a transparent conductive film in this order, in which the photoelectric conversion film contains a compound represented by Formula (1) (hereinafter, referred to as a “specific compound”) and a coloring agent.

The mechanism capable of solving the above problems by adopting such a configuration of the photoelectric conversion element according to the embodiment of the present invention is not always clear, but the present inventors have presumed as follows.

That is, a group represented by A acts as an acceptor, and a moiety represented by “—(Ar¹)ₘ—Ar²—(U¹)ₙ₁” acts as a donor in the molecule of the specific compound. That is, the specific compound has the form in which an acceptor is sandwiched between donors. In such a specific compound, from the viewpoint that the highest occupied molecular orbitals (HOMOs) are likely to be present at both ends, and there is a high probability that the donors are adjacent to each other in the photoelectric conversion film, it is considered that the hole-transportation in the photoelectric conversion film becomes smooth to contribute to the improvement of the sensitivity of the photoelectric conversion element. On the other hand, from the viewpoint that the lowest unoccupied molecular orbital (LUMO) tends to be present at the center of the specific compound due to the acceptor, it is considered that the favorable sensitivity can be obtained. This is because electron migration to an n-type semiconductor material or the like, which may be adjacent to the acceptor, is difficult, but the specific compound has only specific substituents that are less likely to interfere with electron migration. In addition, it is also considered that the fact that the acceptor and the donor are selected from the groups having appropriate electron accepting properties or electron donating properties, respectively, also contributes to the improvement of the sensitivity.

In addition, the photoelectric conversion element according to the embodiment of the present invention has the favorable responsiveness and also has the favorable preventing properties of variation in response.

Hereinafter, the case where the sensitivity, responsiveness, and/or preventing properties of variation in response of the photoelectric conversion element are more excellent is simply referred to as an “effect of the present invention is more excellent”.

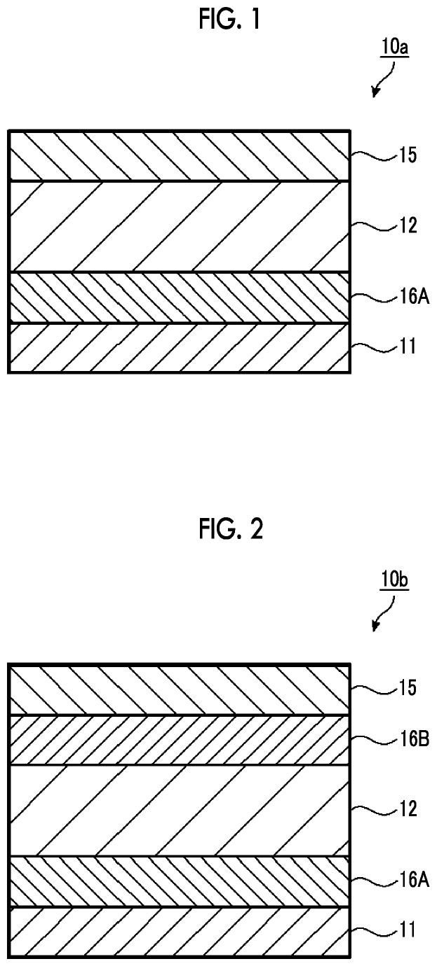

FIG. 1 is a schematic cross-sectional view of one embodiment of a photoelectric conversion element of the present invention.

A photoelectric conversion element 10a illustrated in FIG. 1 has a configuration in which a conductive film (hereinafter, also referred to as a lower electrode) 11 functioning as a lower electrode, an electron blocking film 16A, a photoelectric conversion film 12 containing the specific compound described later, and a transparent conductive film (hereinafter, also referred to as an upper electrode) 15 functioning as an upper electrode are laminated in this order.

FIG. 2 illustrates a configuration example of another photoelectric conversion element. A photoelectric conversion element 10b illustrated in FIG. 2 has a configuration in which the electron blocking film 16A, the photoelectric conversion film 12, a positive hole blocking film 16B, and the upper electrode 15 are laminated on the lower electrode 11 in this order. The lamination order of the electron blocking film 16A, the photoelectric conversion film 12, and the positive hole blocking film 16B in FIGS. 1 and 2 may be appropriately changed according to the application and the characteristics.

In the photoelectric conversion element 10a (or 10b), it is preferable that light is incident on the photoelectric conversion film 12 through the upper electrode 15.

In a case where the photoelectric conversion element 10a (or 10b) is used, a voltage can be applied. In this case, it is preferable that the lower electrode 11 and the upper electrode 15 form a pair of electrodes, and a voltage of 1×10⁻⁵ to 1×10⁷ V/cm is applied between the pair of electrodes. From the viewpoint of the performance and power consumption, the applied voltage is more preferably 1×10⁻⁴ to 1×10⁷ V/cm, and still more preferably 1×10⁻³ to 5×10⁶ V/cm.

Regarding a voltage application method, in FIGS. 1 and 2, it is preferable that the voltage is applied such that the electron blocking film 16A side is a cathode and the photoelectric conversion film 12 side is an anode. In a case where the photoelectric conversion element 10a (or 10b) is used as an optical sensor, or also in a case where the photoelectric conversion element 10a (or 10b) is incorporated in an imaging element, the voltage can be applied by the same method.

As described in detail below, the photoelectric conversion element 10a (or 10b) can be suitably applied to applications of the imaging element.

Hereinafter, the form of each layer constituting the photoelectric conversion element according to the embodiment of the present invention will be described in detail.

[Photoelectric Conversion Film]

The photoelectric conversion film is a film containing a specific compound.

Hereinafter, the specific compound will be described in detail.

<Compound (Specific Compound) Represented by Formula (1)>

The specific compound is a compound represented by Formula (1) described below.

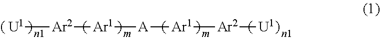

In Formula (1), A is a group represented by any of Formula (2) to Formula (8).

A is preferably a group represented by any of Formula (3) to Formula (8).

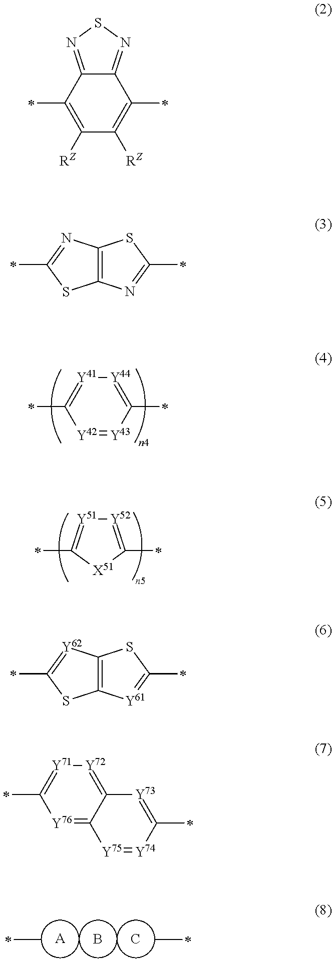

In Formula (2) to Formula (8), * represents a bonding position.

In Formula (2), R^{Z} represents a hydrogen atom or a halogen atom, and a hydrogen atom is preferable.

In Formula (4), n4 represents 1 or 2.

Y⁴¹ to Y⁴⁴ each independently represent —N═ or —CR═.

R represents a hydrogen atom or a substituent (halogen atom or the like).

In a case where n4 is 1, at least one (for example, one to four) of a total of four Y⁴¹ to Y⁴⁴ is —N═ or —CF═.

In a case where n4 is 1, it is preferable that Y⁴¹ and Y⁴⁴ are the same as each other, and Y⁴² and Y⁴³ are the same as each other, and/or it is preferable that Y⁴¹ and Y⁴³ are the same as each other, and Y⁴² and Y⁴⁴ are the same as each other.

In a case where n4 is 2, at least one (for example, one to eight) of a total of eight Y⁴¹ to Y⁴⁴ is —N═ or —CF═.

In a case where n4 is 2, it is preferable that two aromatic rings containing Y⁴¹ to Y⁴⁴ each have one to four Y⁴¹ to Y⁴⁴ of —N═ or —CF═.

In a case where n4 is 2, it is preferable that the two aromatic rings containing Y⁴¹ to Y⁴⁴ are formed in a symmetrical structure. For example, in the following Formula (4P) and/or the following Formula (4Q) showing Formula (4) having the form in which n4 is 2, it is preferable that Y⁴¹ in the left aromatic ring and Y⁴⁴ in the right aromatic ring are the same as each other, Y⁴² in the left aromatic ring and Y⁴³ in the right aromatic ring are the same as each other, Y⁴³ in the left aromatic ring and Y⁴² in the right aromatic ring are the same as each other, and Y⁴⁴ in the left aromatic ring and Y⁴¹ in the right aromatic ring are the same as each other.

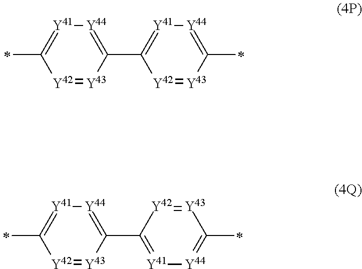

In Formula (5), n5 represents 1 or 2.

Y⁵¹ and Y⁵² each independently represent —N═ or —CR═.

R represents a hydrogen atom or a substituent (halogen atom or the like).

X⁵¹ represents a sulfur atom, an oxygen atom, or a selenium atom.

In Formula (5), in a case where n5 is 1, at least one (one or two) of a total of two Y⁵¹ and Y⁵² is —N═ or —CF═.

In a case where n5 is 1, it is preferable that Y⁵¹ and Y⁵² are the same as each other.

In a case where n5 is 2, at least one (for example, one to four) of a total of four Y⁵¹ and Y⁵² is —N═ or —CF═.

In a case where n5 is 2, it is preferable that two aromatic rings containing Y⁵¹, Y⁵², and X⁵¹ each have one or two Y⁵¹ and Y⁵² of —N═ or —CF═.

In a case where n5 is 2, it is preferable that two aromatic rings containing Y⁵¹, Y⁵², and X⁵¹ are formed in a symmetrical structure. For example, in the following Formula (5P) showing Formula (5) having the form in which n5 is 2, it is preferable that Y⁵¹ in the left aromatic ring and Y⁵² in the right aromatic ring are the same as each other, Y⁵² in the left aromatic ring and Y⁵¹ in the right aromatic ring are the same as each other, and X⁵¹ in the left aromatic ring and X⁵¹ in the right aromatic ring are the same as each other.

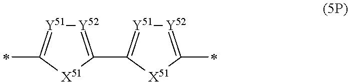

In Formula (6), Y⁶¹ and Y⁶² each independently represent —CR═.

R represents a hydrogen atom or a substituent (halogen atom or the like).

At least one (one or two) of a total of two Y⁶¹ and Y⁶² is —CF═. In addition, it is preferable that Y⁶¹ and Y⁶² are the same as each other.

In Formula (7), Y⁷¹ to Y⁷⁶ each independently represent —N═ or —CR═.

R represents a hydrogen atom or a substituent (halogen atom or the like).

At least one (preferably, one or two) of a total of six Y⁷¹ to Y⁷⁶ is —N═ or —CF═. In addition, it is preferable that Y⁷¹ and Y⁷⁴ each independently represent —CF═, or it is preferable that Y⁷³ and Y⁷⁶ each independently represent —CF═.

In Formula (8), A, B, and C each independently represent an aromatic ring group having 5-membered ring or 6-membered ring.

Examples of the above-described aromatic ring group having 5-membered ring or 6-membered ring include aromatic ring groups having 5-membered ring such as a furan ring group, a pyrrole ring group, a thiophene ring group, an imidazole ring group, a pyrazole ring group, an oxazole ring group, an isoxazole ring group, a thiazole ring group, and an isothiazole ring group; aromatic ring groups having 6-membered ring such as a benzene ring group, a pyridine ring group, a pyrazine ring group, a pyrimidine ring group, a pyridazine ring group, and a triazine ring group. Examples of the above-described aromatic ring group include a thiazole ring group, an isothiazole ring group, a benzene ring group, and a pyridine ring group, or a pyridine ring group is preferable.

A, B, and C are fused with each other to form a fused ring.

“A, B, and C are fused with each other to form a fused ring” means “an aromatic ring group having 5 or 6-membered ring represented by A, an aromatic ring group having 5 or 6-membered ring represented by B, and an aromatic ring group having 5 or 6-membered ring represented by C are fused with one another to form a fused ring”. Specifically, in a case where A, B, and C each represent a benzene ring group, the fused ring formed by A, B, and C being fused is an anthracene ring group.

In Formula (8), at least one (preferably, one or two) of ring member atoms constituting the aromatic ring group represented by A, B, and C is —N═ or —CF═.

As a group represented by Formula (8), any one of a group represented by Formula (9) or a group represented by Formula (10) is preferable.

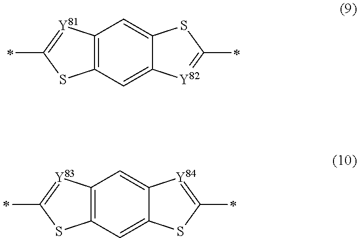

In Formula (9) and Formula (10), * represents a bonding position.

In Formula (9), Y⁸¹ and Y⁸² each independently represent —N═ or —CR═.

R represents a hydrogen atom or a substituent (halogen atom or the like).

At least one (one or two) of a total of two Y⁸¹ and Y⁸² is —N═ or —CF═. In addition, Y⁸¹ and Y⁸² are each preferably —N═.

In Formula (10), Y⁸³ and Y⁸⁴ each independently represent —N═ or —CR═.

R represents a hydrogen atom or a substituent (halogen atom or the like).

At least one (one or two) of a total of two Y⁸³ and Y⁸⁴ is —N═ or —CF═. In addition, Y⁸³ and Y⁸⁴ are each preferably —N═.

In Formula (1), Ar¹ represents an aromatic ring group.

The aromatic ring group represented by Ar¹ may have, as a substituent, a group selected from the group consisting of a halogen atom and an alkyl group having 1 or 2 carbon atoms, which may have a halogen atom.

In a case where the aromatic ring group represented by Ar¹ has the above-described substituent, the number thereof is, for example, 1 to 4.

It is also preferable that the aromatic ring group represented by Ar¹ does not have a substituent.

In Formula (1), Ar² represents an aromatic ring group.

The aromatic ring group represented by Ar² may have, as a substituent other than U¹, a group selected from the group consisting of a halogen atom and an alkyl group having 1 or 2 carbon atoms, which may have a halogen atom.

The above-described substituent (substituent other than U¹), which can be contained in the aromatic ring group represented by Ar², is preferably an alkyl group having 1 or 2 carbon atoms, which may have a halogen atom.

In a case where the aromatic ring group represented by Ar² has the above-described substituent, the number thereof is, for example, 1 to 4.

It is also preferable that the aromatic ring group represented by Ar² does not have the above-described substituent.

In Formula (1), U¹ represents a halogen atom, a cyano group, or an aromatic ring group.

The aromatic ring group represented by U¹ may have, as a substituent, a group selected from the group consisting of a halogen atom, an alkyl group having 1 or 2 carbon atoms, which may have a halogen atom, and a cyano group.

In a case where the aromatic ring group represented by U¹ has a substituent, the number thereof is, for example, 1 to 5.

Examples of the aromatic ring group represented by U¹ include an unsubstituted aromatic ring group, an aromatic ring group having a halogen atom, an aromatic ring group having cyano, and an aromatic ring group having an alkyl group having 1 or 2 carbon atoms, which may have a halogen atom.

In the aromatic ring group having a halogen atom, in a case where the aromatic ring group has a plurality of substituents, at least one (preferably all) of the plurality of substituents may be a halogen atom (fluorine atom and the like). The same applies to the aromatic ring group having cyano and the aromatic ring group having an alkyl group having 1 or 2 carbon atoms, which may have a halogen atom.

Specific examples of the aromatic ring group represented by U¹ include a phenyl group, a fluorophenyl group (4-fluorophenyl group and the like), a difluorophenyl group (3,5-difluorophenyl group and the like), a trifluorophenyl group (3,4,5-difluorophenyl group and the like), a tetrafluorophenyl group, a pentafluorophenyl group, a (trifluoromethyl)phenyl group (4-(trifluoromethyl)phenyl group and the like), a cyanophenyl group (4-cyanophenyl group and the like), and a nitrogen-containing aromatic ring group.

In Formula (1), m represents an integer of 0 to 2.

In Formula (1), n1 represents an integer of 0 to 5, and an integer of 1 to 5 is preferable.

In a case where n1 is 0, there is no U¹ with n1 of 0. For example, in a case where n1 on the left side in Formula (1) is 0, U¹ on the left side does not exist, and Ar¹ on the left side is not bonded to U¹.

(Formula (1-2))

The specific compound is also preferably a compound represented by Formula (1-2).

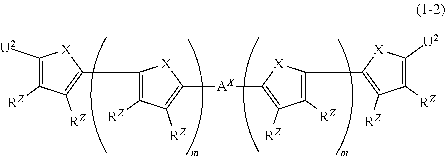

In Formula (1-2), A^{X} is a group represented by Formula (2) or Formula (3).

In Formula (1-2), U² represents a hydrogen atom, a halogen atom, a cyano group, or an aromatic ring group.

The aromatic ring group represented by U² may have, as a substituent, a group selected from the group consisting of a halogen atom, an alkyl group having 1 or 2 carbon atoms, which may have a halogen atom, and a cyano group.

In a case where the aromatic ring group represented by U² has a substituent, the number thereof is, for example, 1 to 4.

Examples of the aromatic ring group represented by U² include the groups described as examples of the aromatic ring group represented by U¹ in Formula (1).

U² is preferably a hydrogen atom or an aromatic ring group.

In Formula (1-2), R^{Z} represents a hydrogen atom or a halogen atom, and a hydrogen atom is preferable.

In Formula (1-2), X represents a sulfur atom, an oxygen atom, or a selenium atom.

In Formula (1-2), m represents an integer of 0 to 2.

(Formula (1-3))

The specific compound is also preferably a compound represented by Formula (1-3).

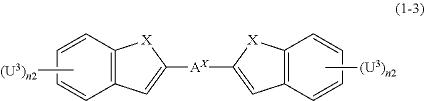

In Formula (1-3), A^{X} is a group represented by Formula (2) or Formula (3).

In Formula (1-3), U³ represents a halogen atom, a cyano group, or an aromatic ring group.

The aromatic ring group represented by U³ may have, as a substituent, a group selected from the group consisting of a halogen atom, an alkyl group having 1 or 2 carbon atoms, which may have a halogen atom, and a cyano group.

In a case where the aromatic ring group represented by U³ has a substituent, the number thereof is, for example, 1 to 5.

Examples of the aromatic ring group represented by U³ include the groups described as examples of the aromatic ring group represented by U¹ in Formula (1).

In Formula (1-3), X represents a sulfur atom, an oxygen atom, or a selenium atom.

In Formula (1-3), n2 represents 0 or 1.

In a case where n2 is 0, there is no U³ with n2 of 0. For example, in a case where n2 on the left side in Formula (1-3) is 0, U³ on the left side does not exist.

(Formula (2-2))

The specific compound is also preferably a compound represented by Formula (2-2).

The compound represented by Formula (2-2) can be referred to as a suitable form of the compound represented by Formula (1-2).

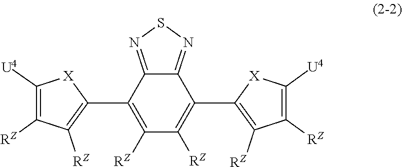

In Formula (2-2), R^{Z} represents a hydrogen atom or a halogen atom, and a hydrogen atom is preferable.

In Formula (2-2), X represents a sulfur atom, an oxygen atom, or a selenium atom.

In Formula (2-2), U⁴ is a fluorine atom, a group represented by Formula (4-1), or a group represented by Formula (4-2).

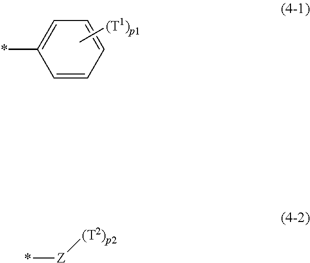

In Formula (4-1), * represents a bonding position.

In Formula (4-1), p1 represents an integer of 1 to 5.

In a case where p1 represents 1, T¹ represents a cyano group. A bonding position of the cyano group may be any one of an ortho-position, a meta-position, or a para-position.

In a case where p1 represents an integer of 2 to 5, T¹ represents a halogen atom. In a case where p1 is within a range of an integer of 2 to 5, p1 is preferably 2 to 4, more preferably 3 to 4, and still more preferably 3.

Specific examples of the group represented by Formula (4-1) include a difluorophenyl group (3,5-difluorophenyl group and the like), a trifluorophenyl group (3,4,5-trifluorophenyl group and the like), and a tetrafluorophenyl group, a pentafluorophenyl group, and a cyanophenyl group (4-cyanophenyl group and the like).

In Formula (4-2), * represents a bonding position.

In Formula (4-2), Z represents a nitrogen-containing aromatic ring group.

In Formula (4-2), p2 represents an integer of 0 to 4.

In a case where p2 is 0, T² does not exist.

In Formula (4-2), T² represents a halogen atom or a cyano group.

In Formula (2-2), U⁴ is preferably a group represented by Formula (4-1) having T¹ that is a cyano group, or preferably a group represented by Formula (4-2).

(Formula (3-2))

The specific compound is also preferably a compound represented by Formula (3-2).

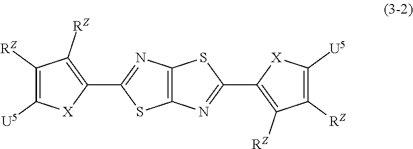

In Formula (3-2), R^{Z} represents a hydrogen atom or a halogen atom, and a hydrogen atom is preferable.

In Formula (3-2), X represents a sulfur atom, an oxygen atom, or a selenium atom.

In Formula (3-2), U⁵ is a fluorine atom, a cyano group, a group represented by Formula (4-3), or a group represented by Formula (4-4).

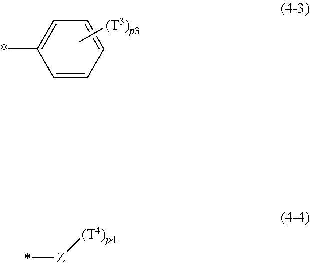

In Formula (4-3), * represents a bonding position.

In Formula (4-3), p3 represents an integer of 1 to 5.

In Formula (4-3), T³ represents a halogen atom or a cyano group.

Examples of the group represented by Formula (4-3) include a group represented by Formula (4-3) having T³ that is a fluorine atom and a group represented by Formula (4-3) having T³ that is a cyano group.

In the group represented by Formula (4-3) having T³ that is a fluorine atom, in a case where a plurality of T³'s exist, at least one (preferably all) of the plurality of T³'s may be a fluorine atom. The same applies to the group represented by Formula (4-3) having T³ that is a cyano group.

Specific examples of the group represented by Formula (4-3) include a fluorophenyl group (4-fluorophenyl group and the like), a difluorophenyl group (3,5-difluorophenyl group and the like), a trifluorophenyl group (3,4,5-difluorophenyl group and the like), and a tetrafluorophenyl group, a pentafluorophenyl group, and a cyanophenyl group (4-cyanophenyl group and the like).

In Formula (4-4), * represents a bonding position.

In Formula (4-4), Z represents a nitrogen-containing aromatic ring group.

In Formula (4-4), p4 represents an integer of 0 to 4.

In a case where p4 is 0, T⁴ does not exist.

In Formula (4-4), T⁴ represents a halogen atom, an alkyl group having 1 or 2 carbon atoms, which may have a halogen atom, or a cyano group.

In Formula (3-2), U⁵ is preferably a group represented by Formula (4-3) or a group represented by Formula (4-4), and a group represented by Formula (4-4) is more preferable.

(Formula (3-3))

The specific compound is also preferably a compound represented by Formula (3-3).

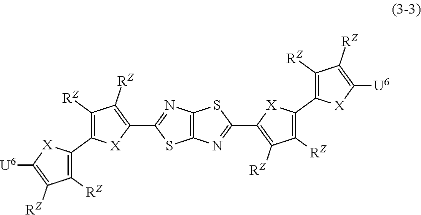

In Formula (3-3), R^{Z} represents a hydrogen atom or a halogen atom, and a hydrogen atom is preferable.

In Formula (3-3), X represents a sulfur atom, an oxygen atom, or a selenium atom.

In Formula (3-3), U⁶ is a fluorine atom, a cyano group, a group represented by Formula (4-5), or a group represented by Formula (4-6).

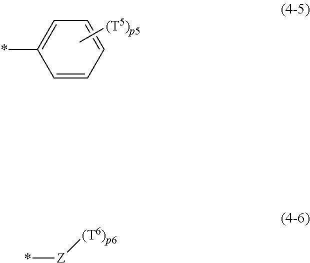

In Formula (4-5), * represents a bonding position.

In Formula (4-5), p5 represents an integer of 0 to 5.

In Formula (4-5), T⁵ represents a halogen atom, an alkyl group having 1 or 2 carbon atoms, which may have a halogen atom, or a cyano group.

Examples of the group represented by Formula (4-5) include a group represented by Formula (4-5) having T⁵ that is a fluorine atom, a group represented by Formula (4-5) having T⁵ that is a cyano group, a group represented by Formula (4-5) having T⁵ of an alkyl group having 1 or 2 carbon atoms, which may have a halogen atom.

In the group represented by Formula (4-5) having T⁵ that is a fluorine atom, in a case where a plurality of T⁵'s exist, at least one (preferably all) of the plurality of T⁵'s may be a fluorine atom. The same applies to a group represented by Formula (4-5) having T⁵ that is a cyano group, and a group represented by Formula (4-5) having T⁵ of an alkyl group having 1 or 2 carbon atoms, which may have a halogen atom.

Specific examples of the group represented by Formula (4-5) include a phenyl group, a fluorophenyl group (4-fluorophenyl group and the like), a difluorophenyl group (3,5-difluorophenyl group and the like), a trifluorophenyl group (3,4,5-difluorophenyl group and the like), a tetrafluorophenyl group, a pentafluorophenyl group, a trifluoromethylphenyl group (4-(trifluoromethyl)phenyl group and the like), and a cyanophenyl group (4-cyanophenyl group and the like).

In Formula (4-6), * represents a bonding position.

In Formula (4-6), Z represents a nitrogen-containing aromatic ring group.

In Formula (4-6), p4 represents an integer of 0 to 4.

In a case where p4 is 0, T⁶ does not exist.

In Formula (4-6), T⁶ represents a halogen atom, an alkyl group having 1 or 2 carbon atoms, which may have a halogen atom, or a cyano group.

In Formula (3-3), U⁶ is preferably a cyano group, a group represented by Formula (4-5) having T⁵ that is a fluorine atom, a group represented by Formula (4-5) having T⁵ that is a cyano group, or a group represented by Formula (4-6), more preferably a group represented by Formula (4-5) having T⁵ that is a fluorine atom, a group represented by Formula (4-5) having T⁵ that is a cyano group, or a group represented by Formula (4-6), and still more preferably a group represented by Formula (4-5) having T⁵ that is a cyano group, or a group represented by Formula (4-6).

(Formula (3-4))

The specific compound is also preferably a compound represented by Formula (3-4).

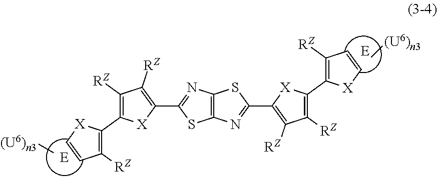

In Formula (3-4), R^{Z} represents a hydrogen atom or a halogen atom, and a hydrogen atom is preferable.

In Formula (3-4), X represents a sulfur atom, an oxygen atom, or a selenium atom.

In Formula (3-4), E represents an aromatic ring.

The aromatic ring represented by E is fused with a five-membered ring bonded to one R^{Z}, and shares the five-membered ring and two carbon atoms as ring member atoms.

The aromatic ring represented by E may have a halogen atom as a substituent other than U⁶.

The above-described substituent (substituent other than U⁶) that can be contained in the aromatic ring represented by E is preferably a halogen atom other than a fluorine atom.

In a case where the aromatic ring represented by E has the above-described substituent, the number thereof is, for example, 1 to 3.

It is also preferable that the aromatic ring represented by E does not have the above-described substituent.

In Formula (3-4), n3 represents an integer of 0 to 4.

In a case where n3 is 0, there is no U⁶ with n3 of 0. For example, in a case where n3 on the left side in Formula (3-4) is 0, U⁶ on the left side does not exist, and E on the left side is not bonded to U⁶.

In Formula (3-4), U⁶ is a fluorine atom, a cyano group, a group represented by Formula (4-5), or a group represented by Formula (4-6). Formula (4-5) and Formula (4-6) are as described above.

(Formula (3-5))

The specific compound is also preferably a compound represented by Formula (3-5).

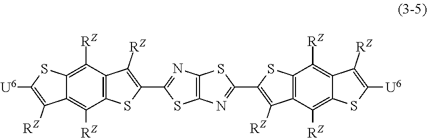

In Formula (3-5), R^{Z} represents a hydrogen atom or a halogen atom, and a hydrogen atom is preferable.

In Formula (3-5), U⁶ is a fluorine atom, a cyano group, a group represented by Formula (4-5), or a group represented by Formula (4-6). Formula (4-5) and Formula (4-6) are as described above.

(Formula (3-6))

The specific compound is also preferably a compound represented by Formula (3-6).

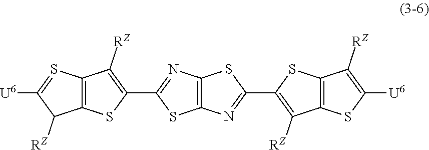

In Formula (3-6), R^{Z} represents a hydrogen atom or a halogen atom, and a hydrogen atom is preferable.

In Formula (3-6), U⁶ is a fluorine atom, a cyano group, a group represented by Formula (4-5), or a group represented by Formula (4-6). Formula (4-5) and Formula (4-6) are as described above.

In Formula (3-6), U⁶ is preferably a group represented by Formula (4-5) having T⁵ that is a fluorine atom, a group represented by Formula (4-5) having T⁵ that is a cyano group, or a group represented by Formula (4-6), and more preferably a group represented by Formula (4-6).

(Formula (1-4))

The specific compound is also preferably a compound represented by Formula (1-4).

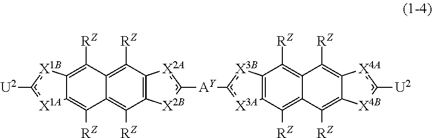

In Formula (1-4), A^{Y} is a group represented by any of Formula (2) to Formula (8).

In Formula (1-4), U² represents a hydrogen atom, a halogen atom, a cyano group, or an aromatic ring group.

The aromatic ring group represented by U² may have, as a substituent, a group selected from the group consisting of a halogen atom, an alkyl group having 1 or 2 carbon atoms, which may have a halogen atom, and a cyano group.

In a case where the aromatic ring group represented by U² has a substituent, the number thereof is, for example, 1 to 4.

Examples of the aromatic ring group represented by U² include the groups described as examples of the aromatic ring group represented by U¹ in Formula (1).

In Formula (1-4), R^{Z} represents a hydrogen atom or a halogen atom, and a hydrogen atom is preferable.

In Formula (1-4), one of X^{1A} and X^{1B} represents a sulfur atom, an oxygen atom, or a selenium atom, and another of X^{1A} and X^{1B} represents —CR═.

In Formula (1-4), one of X^{2A} and X^{2B} represents a sulfur atom, an oxygen atom, or a selenium atom, and another of X^{2A} and X^{2B} represents —CR═.

In Formula (1-4), one of X^{3A} and X^{3B} represents a sulfur atom, an oxygen atom, or a selenium atom, and another of X^{3A} and X^{3B} represents —CR═.

In Formula (1-4), one of X^{4A} and X^{4B} represents a sulfur atom, an oxygen atom, or a selenium atom, and another of X^{4A} and X^{4B} represents —CR═.

R represents a hydrogen atom or a substituent (halogen atom or the like).

(Formula (1-5))

The specific compound is also preferably a compound represented by Formula (1-5).

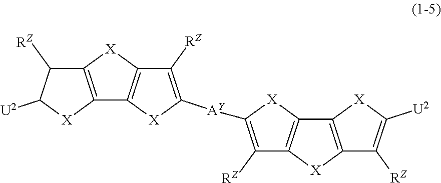

In Formula (1-5), A^{Y} is a group represented by any of Formula (2) to Formula (8).

In Formula (1-5), U² represents a hydrogen atom, a halogen atom, a cyano group, or an aromatic ring group.

The aromatic ring group represented by U² may have, as a substituent, a group selected from the group consisting of a halogen atom, an alkyl group having 1 or 2 carbon atoms, which may have a halogen atom, and a cyano group.

In a case where the aromatic ring group represented by U² has a substituent, the number thereof is, for example, 1 to 4.

Examples of the aromatic ring group represented by U² include the groups described as examples of the aromatic ring group represented by U¹ in Formula (1).

In Formula (1-5), R^{Z} represents a hydrogen atom or a halogen atom, and a hydrogen atom is preferable.

In Formula (1-5), X represents a sulfur atom, an oxygen atom, or a selenium atom.

(Formula (1-6))

The specific compound is also preferably a compound represented by Formula (1-6).

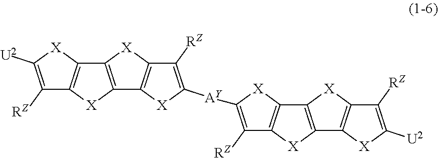

In Formula (1-6), A^{Y} is a group represented by any of Formula (2) to Formula (8).

In Formula (1-6), U² represents a hydrogen atom, a halogen atom, a cyano group, or an aromatic ring group.

The aromatic ring group represented by U² may have, as a substituent, a group selected from the group consisting of a halogen atom, an alkyl group having 1 or 2 carbon atoms, which may have a halogen atom, and a cyano group.

In a case where the aromatic ring group represented by U² has a substituent, the number thereof is, for example, 1 to 4.

Examples of the aromatic ring group represented by U² include the groups described as examples of the aromatic ring group represented by U¹ in Formula (1).

In Formula (1-6), R^{Z} represents a hydrogen atom or a halogen atom, and a hydrogen atom is preferable.

In Formula (1-6), X represents a sulfur atom, an oxygen atom, or a selenium atom.

(Formula (1-7))

The specific compound is also preferably a compound represented by Formula (1-7).

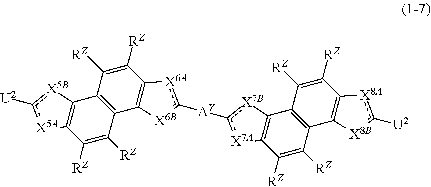

In Formula (1-7), A^{Y} is a group represented by any of Formula (2) to Formula (8).

In Formula (1-7), U² represents a hydrogen atom, a halogen atom, a cyano group, or an aromatic ring group.

The aromatic ring group represented by U² may have, as a substituent, a group selected from the group consisting of a halogen atom, an alkyl group having 1 or 2 carbon atoms, which may have a halogen atom, and a cyano group.

In a case where the aromatic ring group represented by U² has a substituent, the number thereof is, for example, 1 to 4.

Examples of the aromatic ring group represented by U² include the groups described as examples of the aromatic ring group represented by U¹ in Formula (1).

In Formula (1-7), R^{Z} represents a hydrogen atom or a halogen atom, and a hydrogen atom is preferable.

In Formula (1-7), one of X^{5A} and X^{5B} represents a sulfur atom, an oxygen atom, or a selenium atom, and another of X^{5A} and X^{5B} represents —CR═.

In Formula (1-7), one of X^{6A} and X^{6B} represents a sulfur atom, an oxygen atom, or a selenium atom, and another of X^{6A} and X^{6B} represents —CR═.

In Formula (1-7), one of X^{7A} and X^{7B} represents a sulfur atom, an oxygen atom, or a selenium atom, and another of X^{7A} and X^{7B} represents —CR═.

In Formula (1-7), one of X^{8A} and X^{8B} represents a sulfur atom, an oxygen atom, or a selenium atom, and another of X^{8A} and X^{8B} represents —CR═.

R represents a hydrogen atom or a substituent (halogen atom or the like).

(Formula (1-8))

The specific compound is also preferably a compound represented by Formula (1-8).

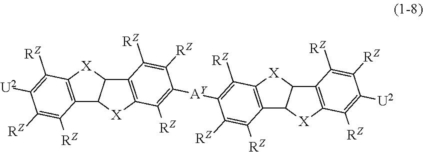

In Formula (1-8), A^{Y} is a group represented by any of Formula (2) to Formula (8).

In Formula (1-8), U² represents a hydrogen atom, a halogen atom, a cyano group, or an aromatic ring group.

The aromatic ring group represented by U² may have, as a substituent, a group selected from the group consisting of a halogen atom, an alkyl group having 1 or 2 carbon atoms, which may have a halogen atom, and a cyano group.

In a case where the aromatic ring group represented by U² has a substituent, the number thereof is, for example, 1 to 4.

Examples of the aromatic ring group represented by U² include the groups described as examples of the aromatic ring group represented by U¹ in Formula (1).

In Formula (1-8), R^{Z} represents a hydrogen atom or a halogen atom, and a hydrogen atom is preferable.

In Formula (1-8), X represents a sulfur atom, an oxygen atom, or a selenium atom.

It is also preferable that the specific compound is formed in a bilaterally symmetrical structure.

In the specific compound, for example, it is also preferable that two groups represented by “—(Ar¹)ₘ—Ar²—(U¹)ₙ₁” existing in Formula (1) have the same structure.

Specific examples of the specific compound will be described below.

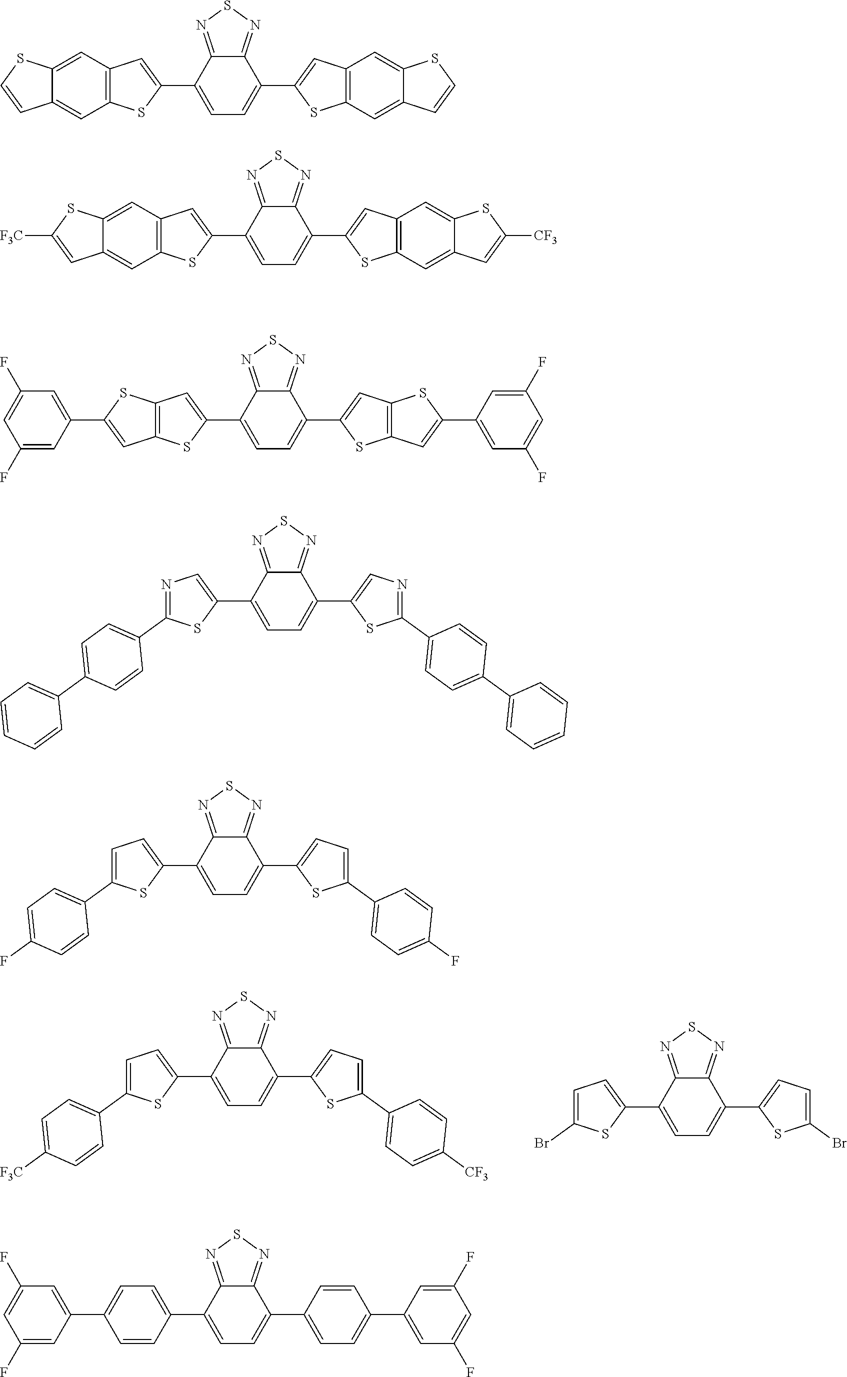

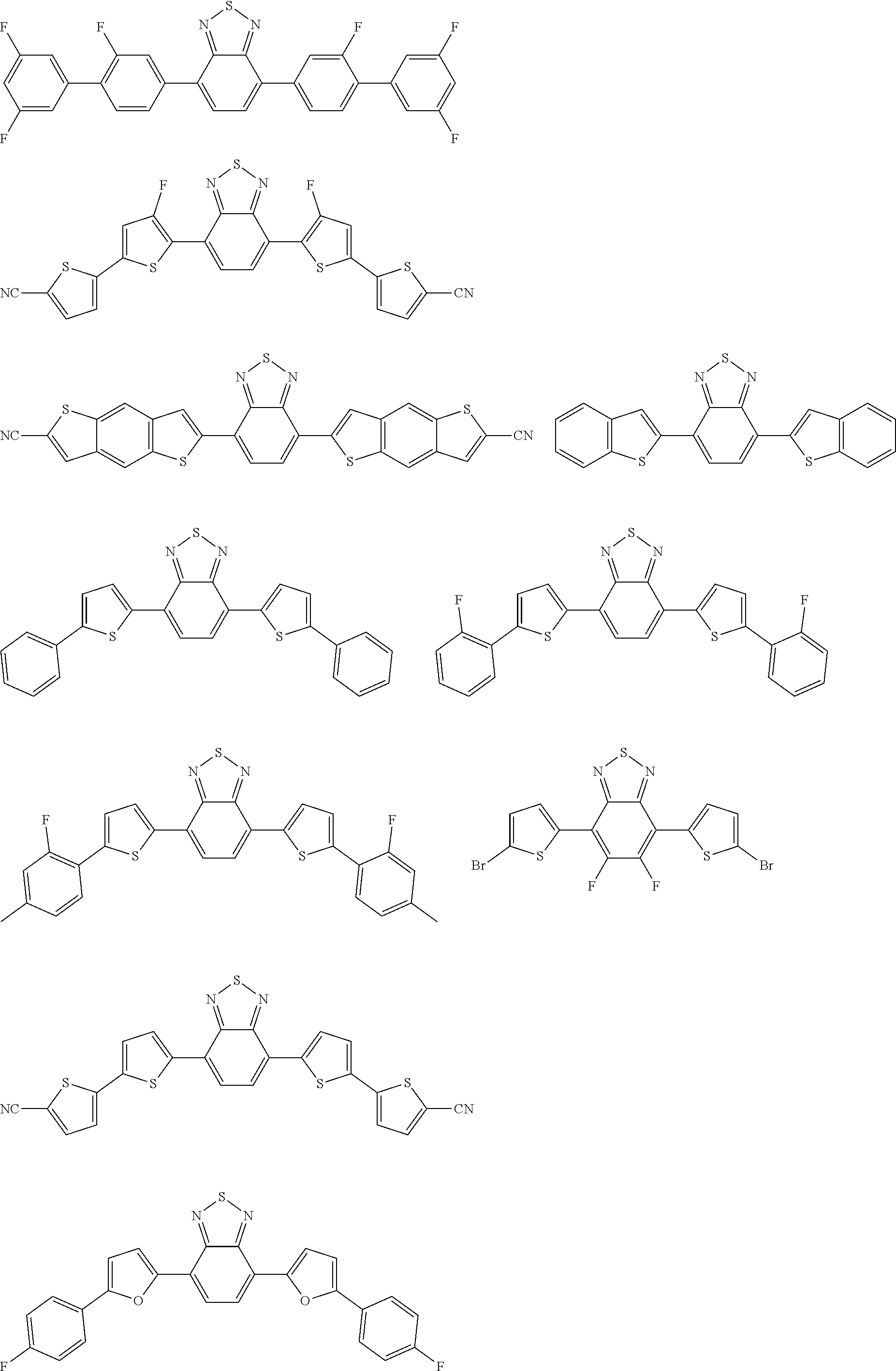

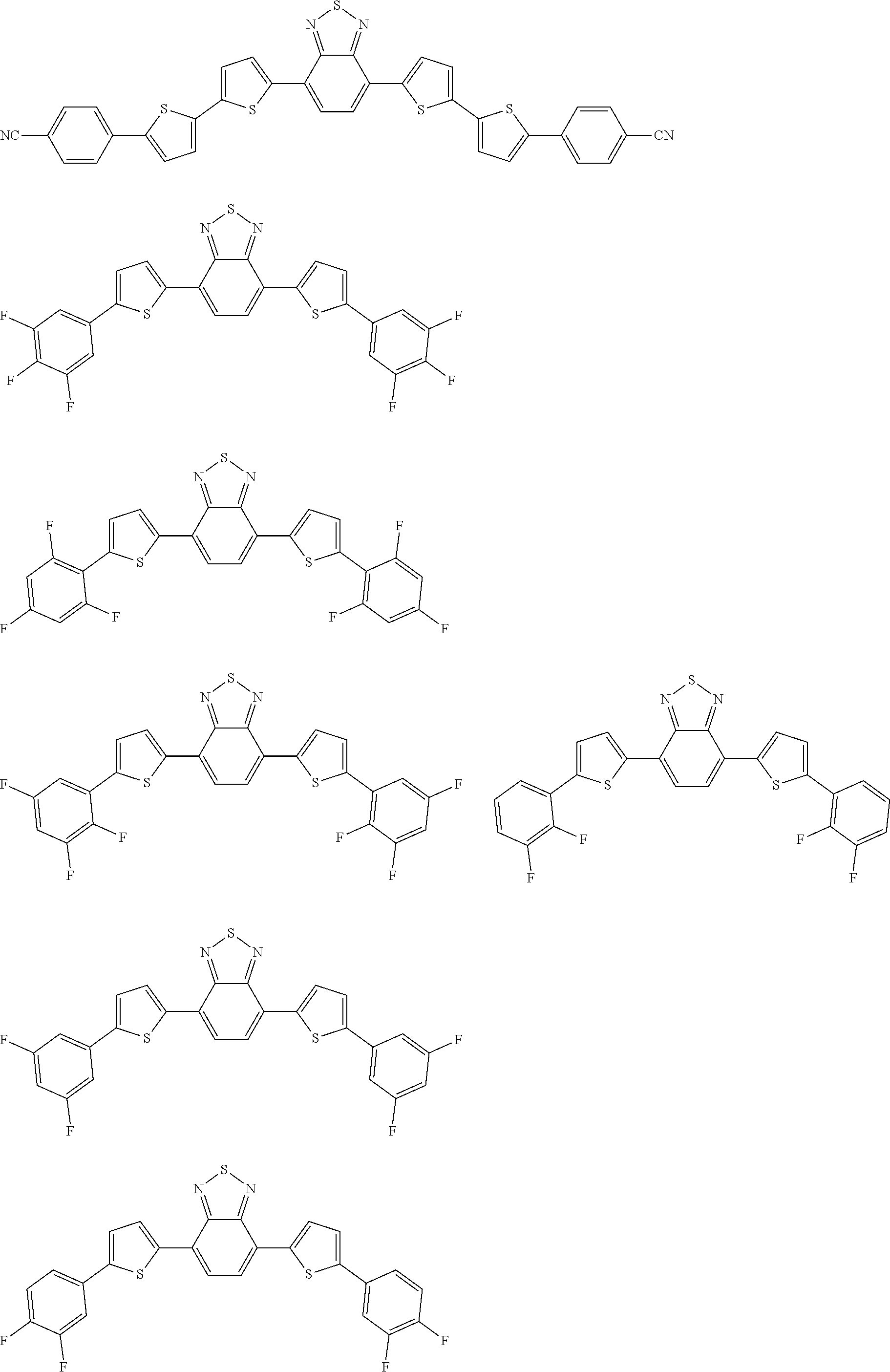

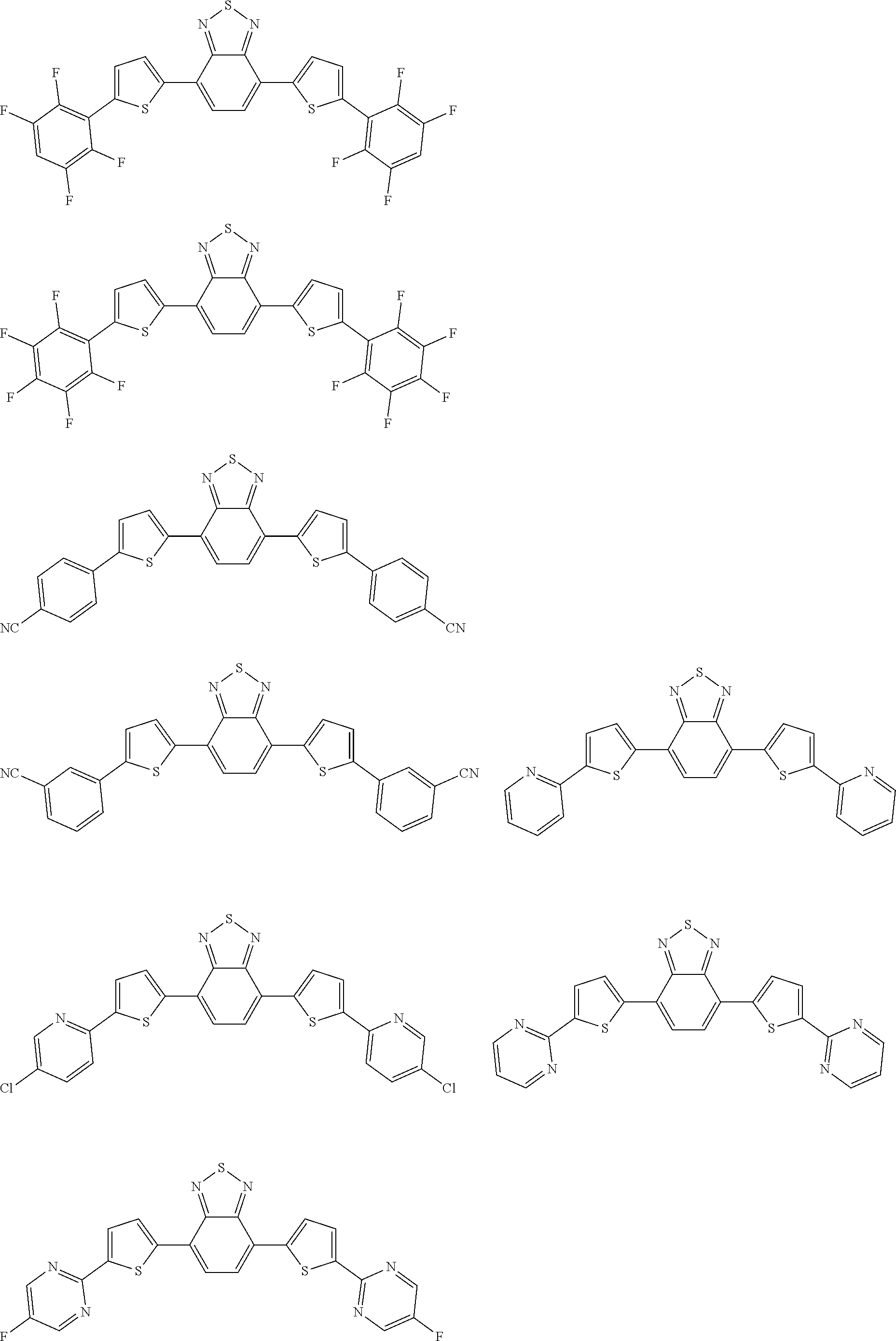

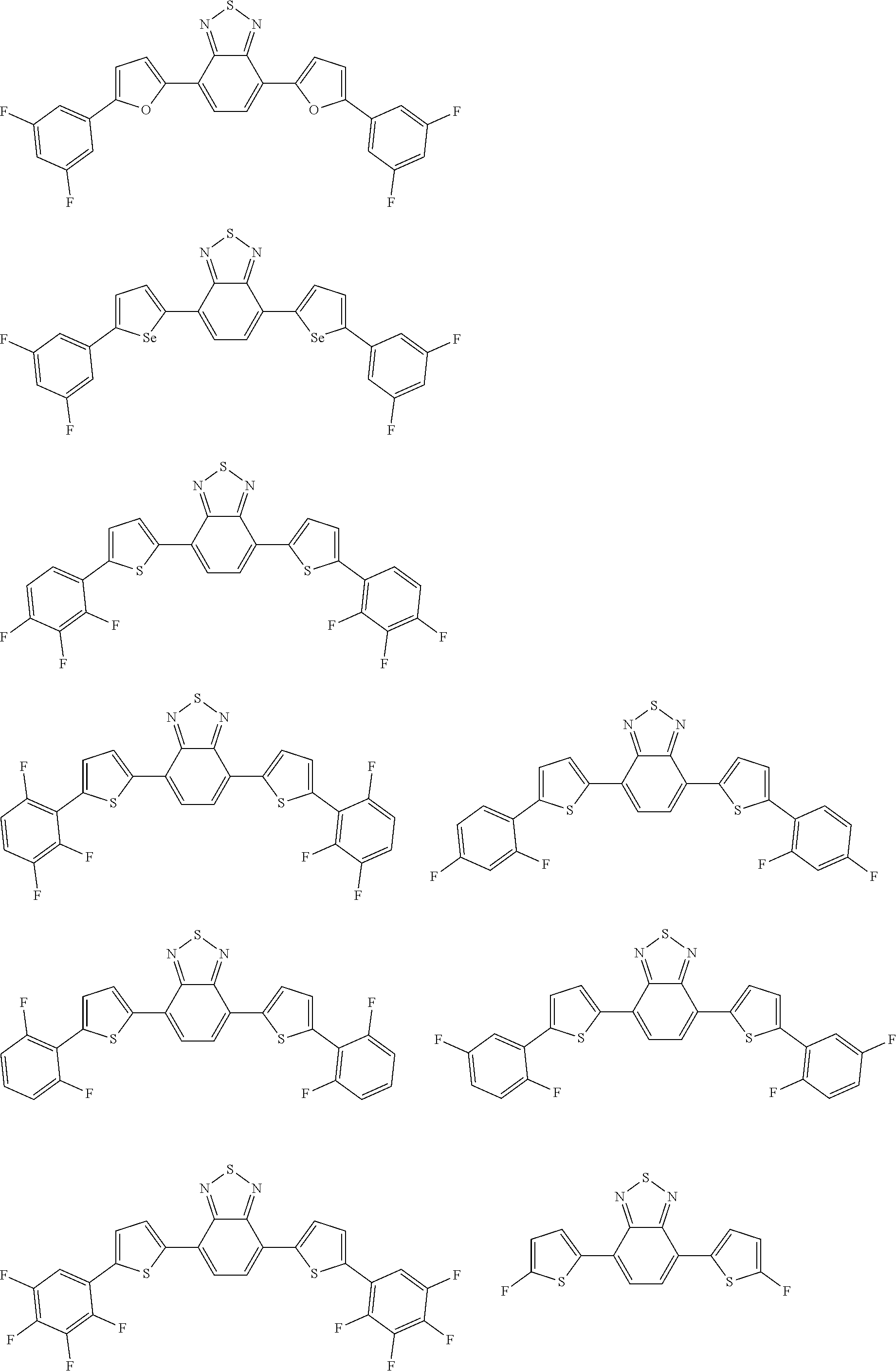

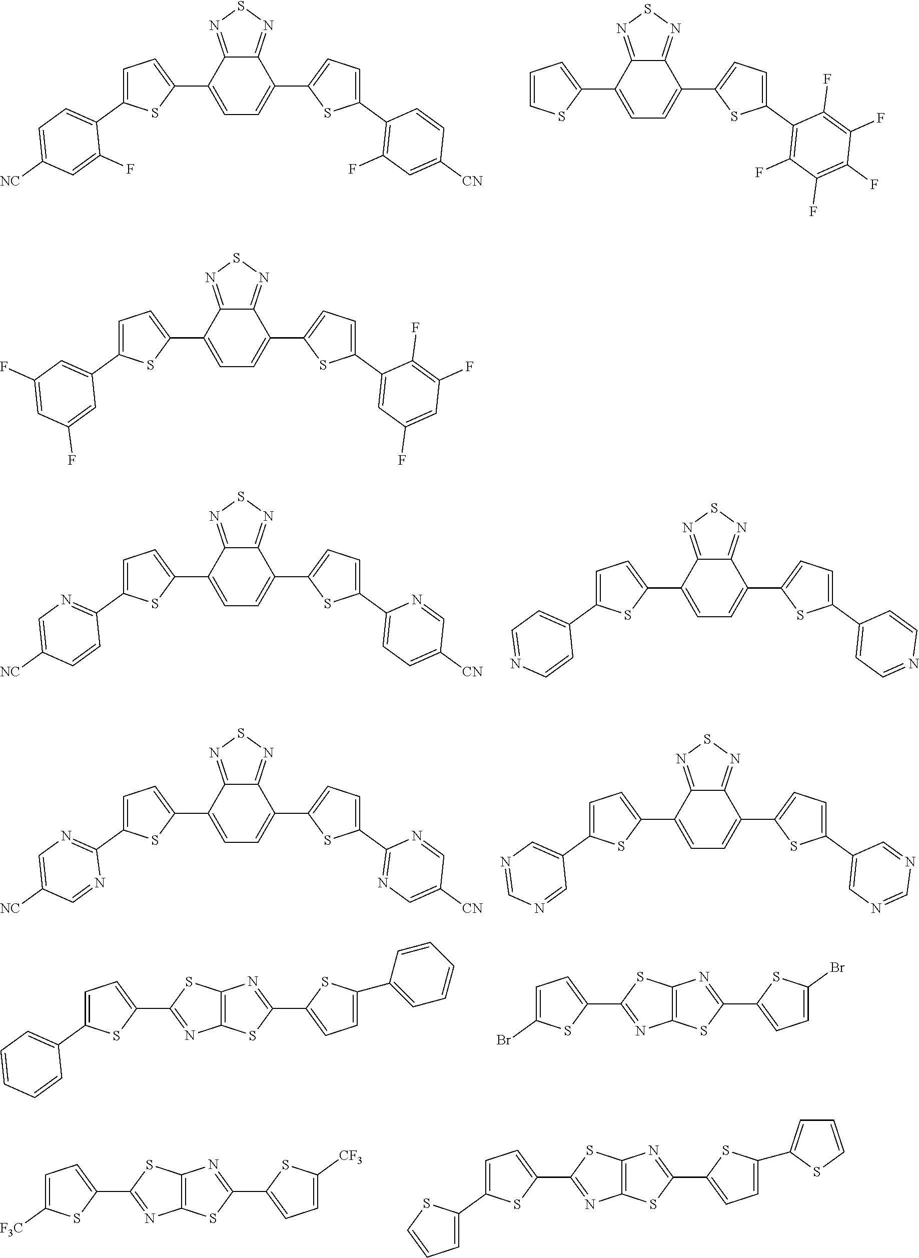

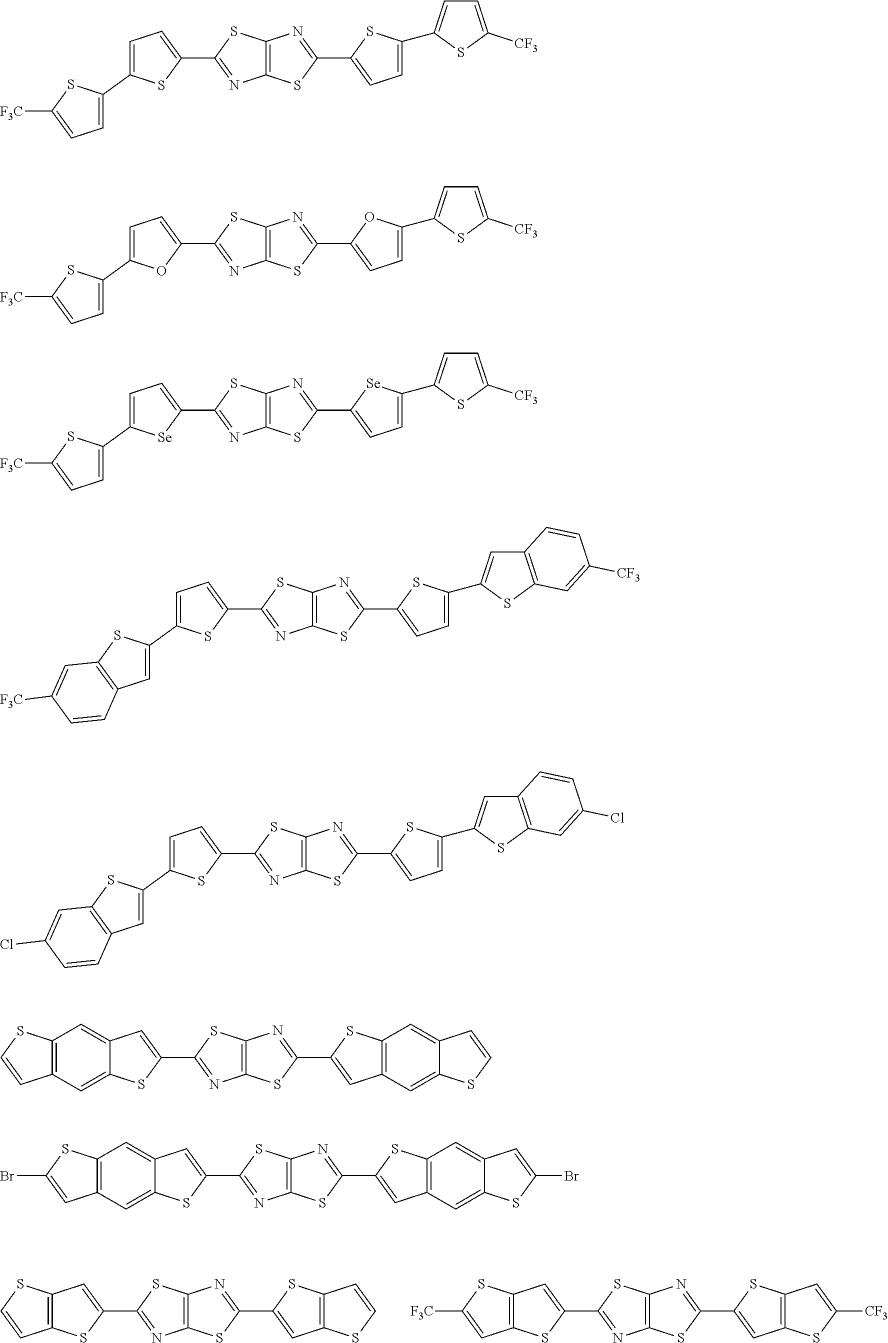

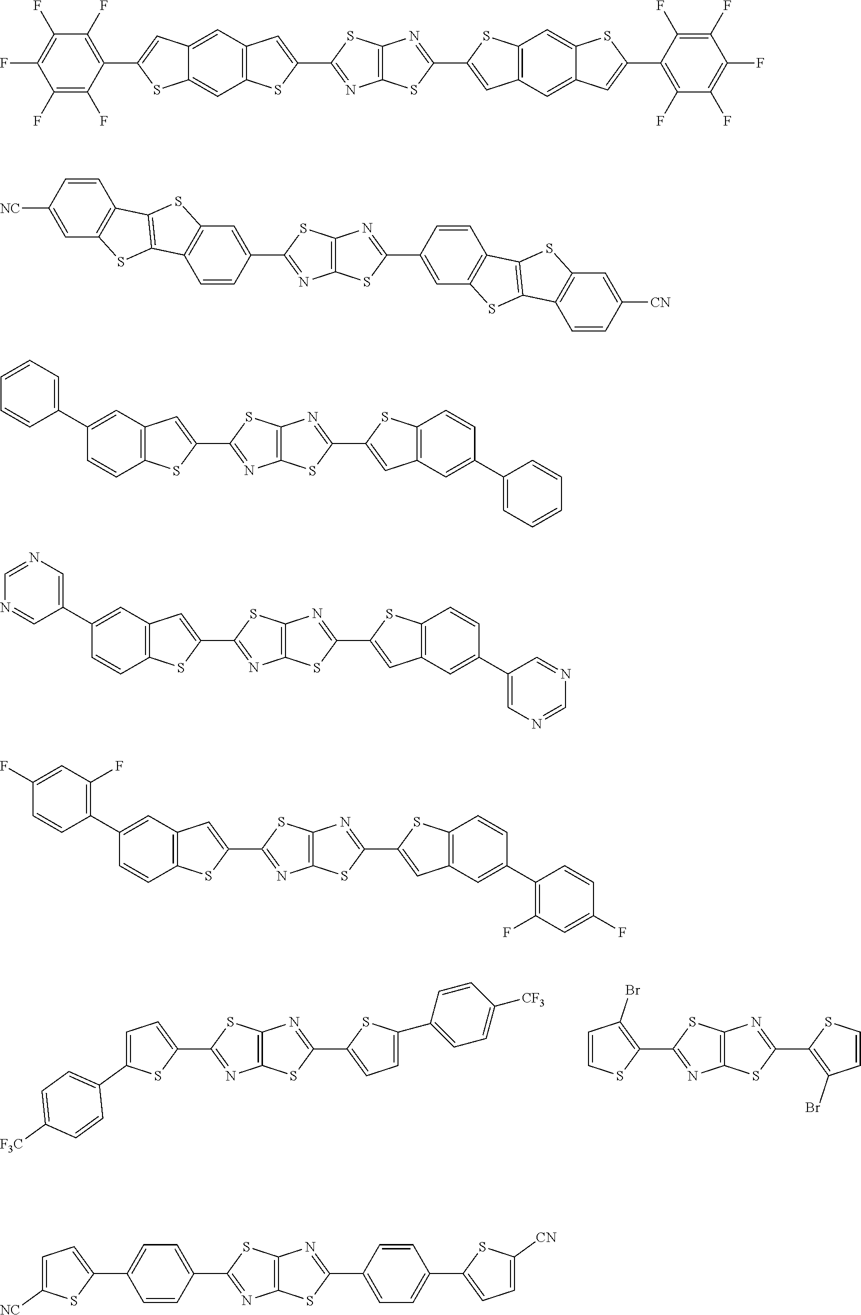

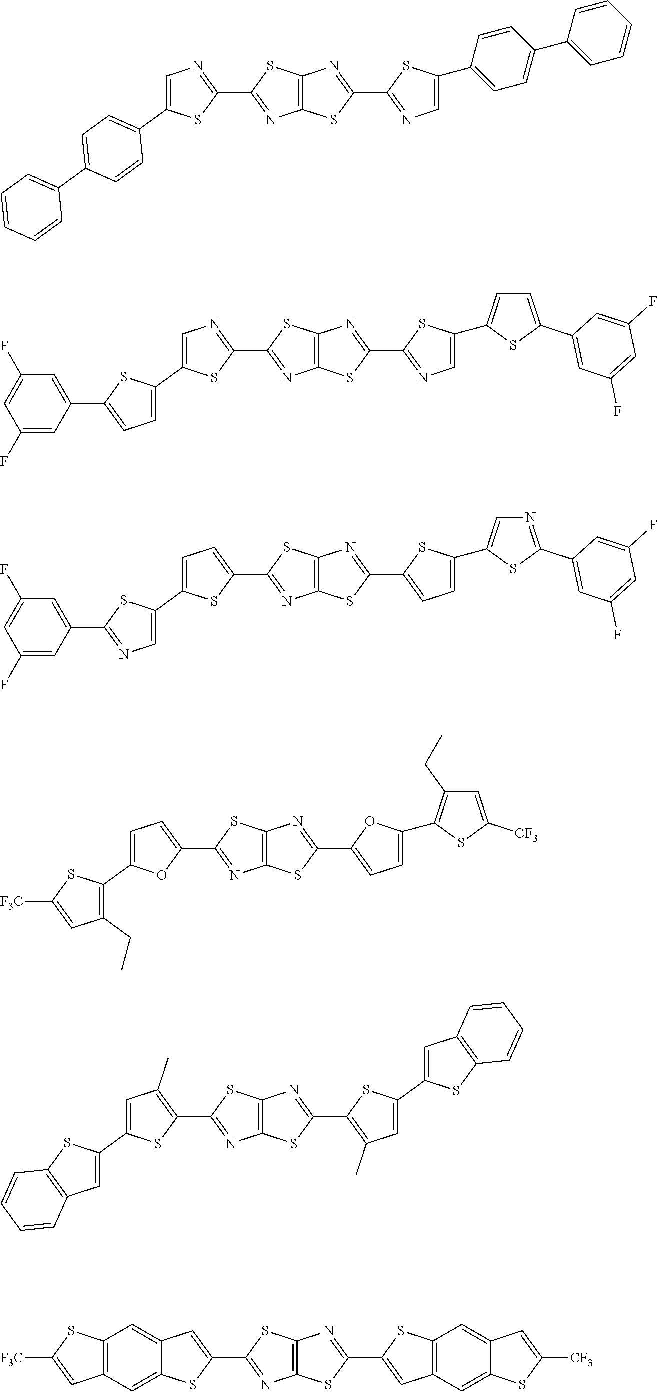 [SMILES: c1ccc(-c2ccc(-c3cnc(-c4nc5sc(-c6ncc(-c7ccc(-c8ccccc8)cc7)s6)nc5s4)s3)cc2)cc1 ; Fc1cc(F)cc(-c2ccc(-c3cnc(-c4nc5sc(-c6ncc(-c7ccc(-c8cc(F)cc(F)c8)s7)s6)nc5s4)s3)s2)c1 ; Fc1cc(F)cc(-c2ncc(-c3ccc(-c4nc5sc(-c6ccc(-c7cnc(-c8cc(F)cc(F)c8)s7)s6)nc5s4)s3)s2)c1 ; CCc1cc(C(F)(F)F)sc1-c1ccc(-c2nc3sc(-c4ccc(-c5sc(C(F)(F)F)cc5CC)o4)nc3s2)o1 ; Cc1cc(-c2cc3ccccc3s2)sc1-c1nc2sc(-c3sc(-c4cc5ccccc5s4)cc3C)nc2s1 ; FC(F)(F)c1cc2cc3sc(-c4nc5sc(-c6cc7cc8sc(C(F)(F)F)cc8cc7s6)nc5s4)cc3cc2s1]

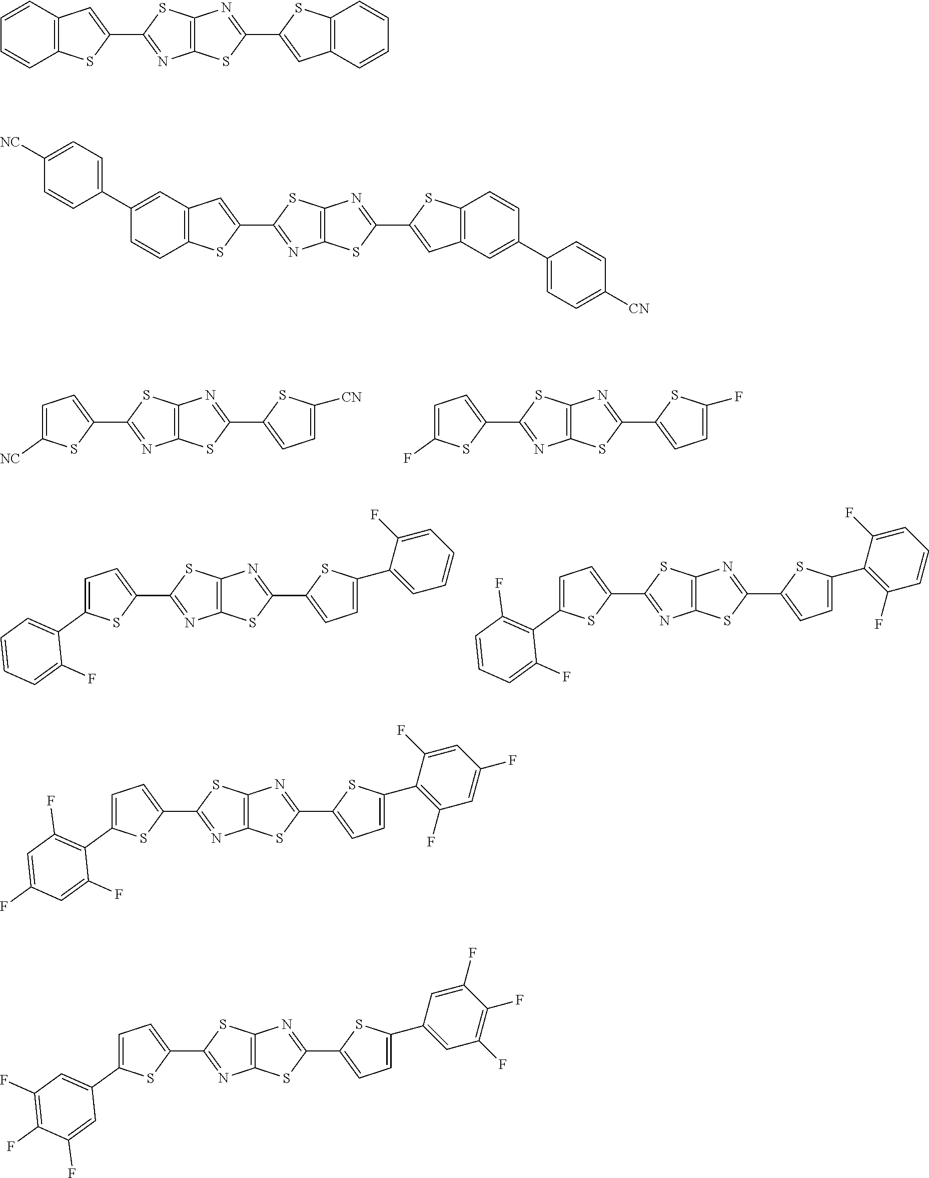

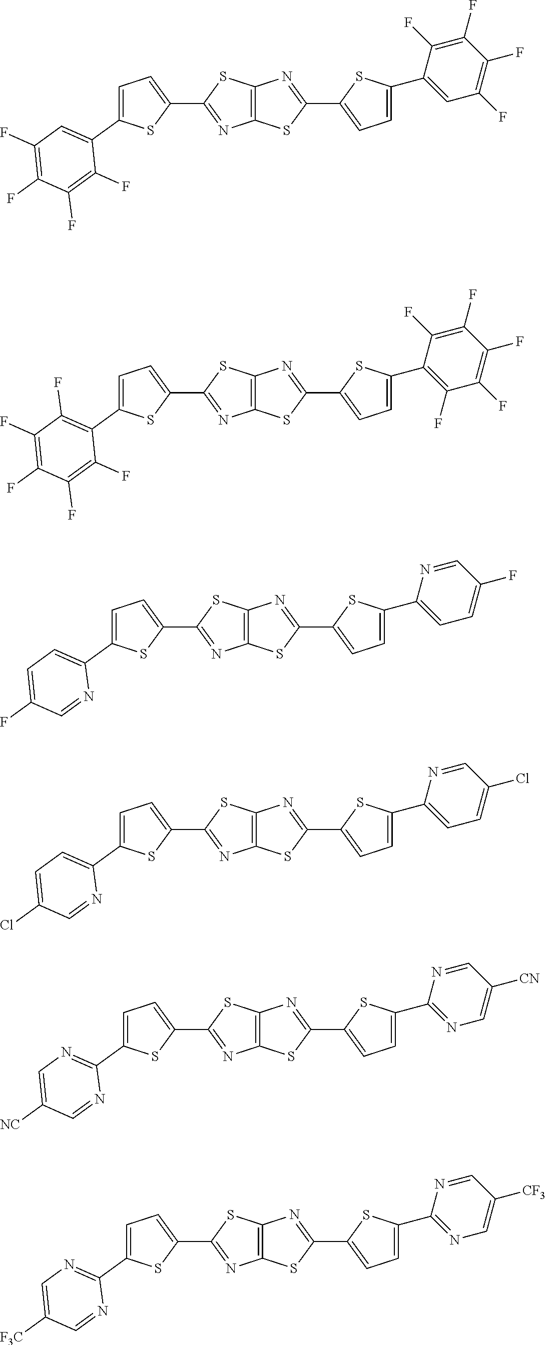 [SMILES: Fc1cc(-c2ccc(-c3nc4sc(-c5ccc(-c6cc(F)c(F)c(F)c6F)s5)nc4s3)s2)c(F)c(F)c1F ; Fc1c(F)c(F)c(-c2ccc(-c3nc4sc(-c5ccc(-c6c(F)c(F)c(F)c(F)c6F)s5)nc4s3)s2)c(F)c1F ; Fc1ccc(-c2ccc(-c3nc4sc(-c5ccc(-c6ccc(F)cn6)s5)nc4s3)s2)nc1 ; Clc1ccc(-c2ccc(-c3nc4sc(-c5ccc(-c6ccc(Cl)cn6)s5)nc4s3)s2)nc1 ; N#Cc1cnc(-c2ccc(-c3nc4sc(-c5ccc(-c6ncc(C#N)cn6)s5)nc4s3)s2)nc1 ; FC(F)(F)c1cnc(-c2ccc(-c3nc4sc(-c5ccc(-c6ncc(C(F)(F)F)cn6)s5)nc4s3)s2)nc1]

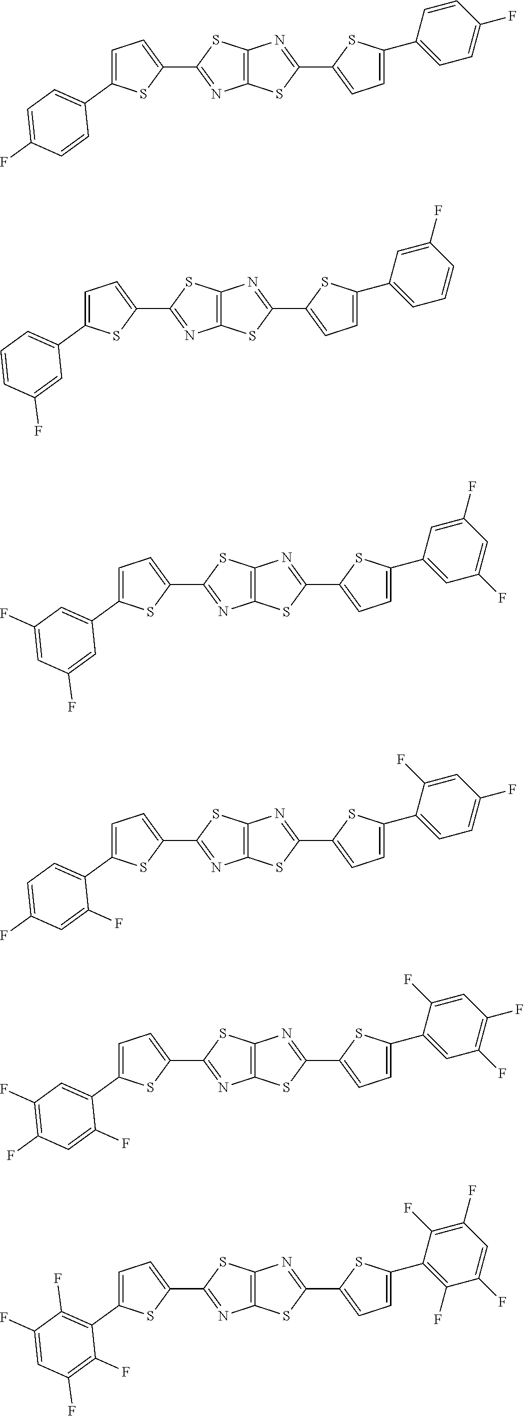 [SMILES: Fc1ccc(-c2ccc(-c3nc4sc(-c5ccc(-c6ccc(F)cc6)s5)nc4s3)s2)cc1 ; Fc1cccc(-c2ccc(-c3nc4sc(-c5ccc(-c6cccc(F)c6)s5)nc4s3)s2)c1 ; Fc1cc(F)cc(-c2ccc(-c3nc4sc(-c5ccc(-c6cc(F)cc(F)c6)s5)nc4s3)s2)c1 ; Fc1ccc(-c2ccc(-c3nc4sc(-c5ccc(-c6ccc(F)cc6F)s5)nc4s3)s2)c(F)c1 ; Fc1cc(F)c(-c2ccc(-c3nc4sc(-c5ccc(-c6cc(F)c(F)cc6F)s5)nc4s3)s2)cc1F ; Fc1cc(F)c(F)c(-c2ccc(-c3nc4sc(-c5ccc(-c6c(F)c(F)cc(F)c6F)s5)nc4s3)s2)c1F]

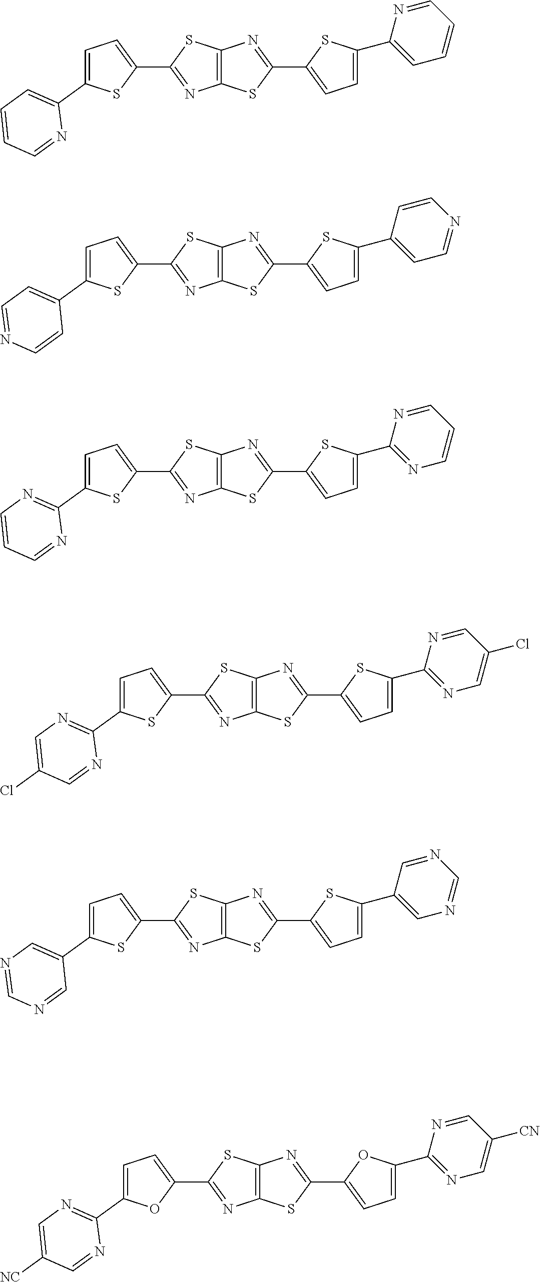 [SMILES: c1ccc(-c2ccc(-c3nc4sc(-c5ccc(-c6ccccn6)s5)nc4s3)s2)nc1 ; c1cc(-c2ccc(-c3nc4sc(-c5ccc(-c6ccncc6)s5)nc4s3)s2)ccn1 ; c1cnc(-c2ccc(-c3nc4sc(-c5ccc(-c6ncccn6)s5)nc4s3)s2)nc1 ; Clc1cnc(-c2ccc(-c3nc4sc(-c5ccc(-c6ncc(Cl)cn6)s5)nc4s3)s2)nc1 ; c1ncc(-c2ccc(-c3nc4sc(-c5ccc(-c6cncnc6)s5)nc4s3)s2)cn1 ; N#Cc1cnc(-c2ccc(-c3nc4sc(-c5ccc(-c6ncc(C#N)cn6)o5)nc4s3)o2)nc1]

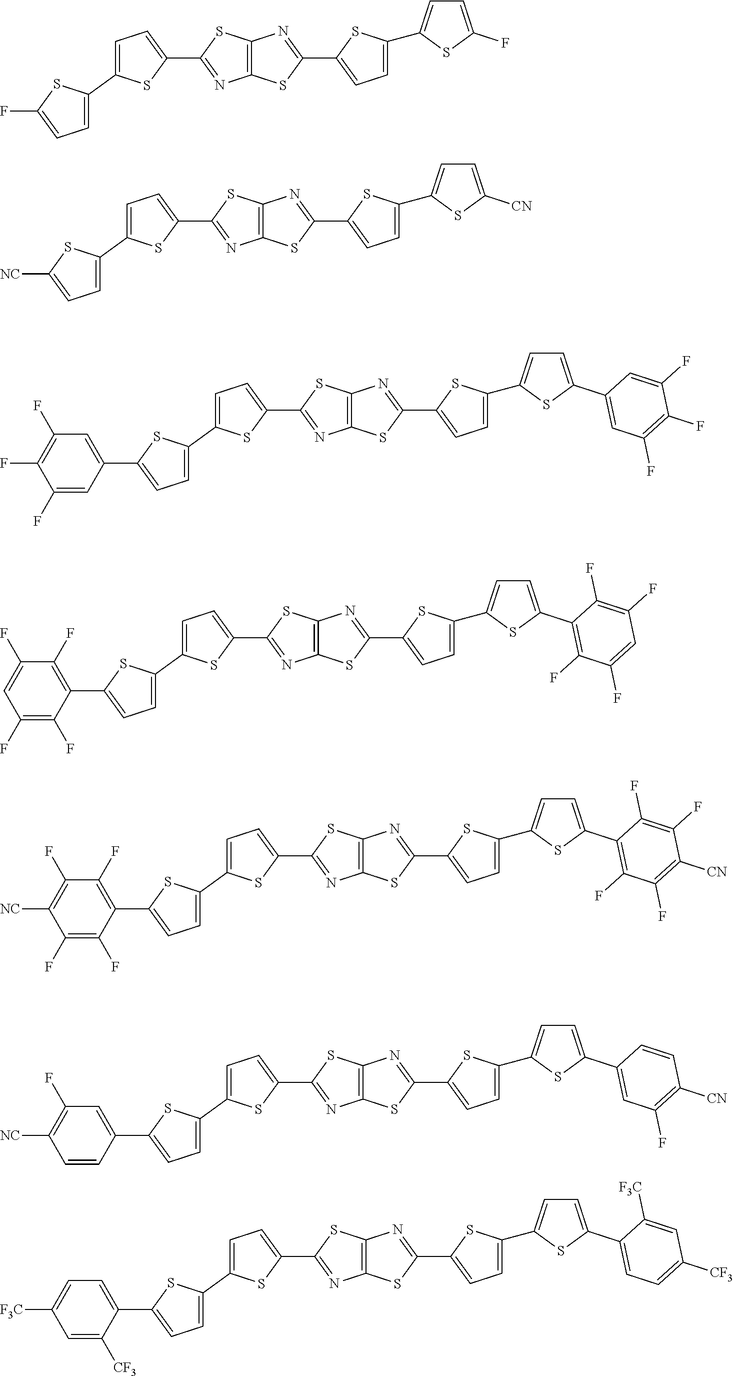

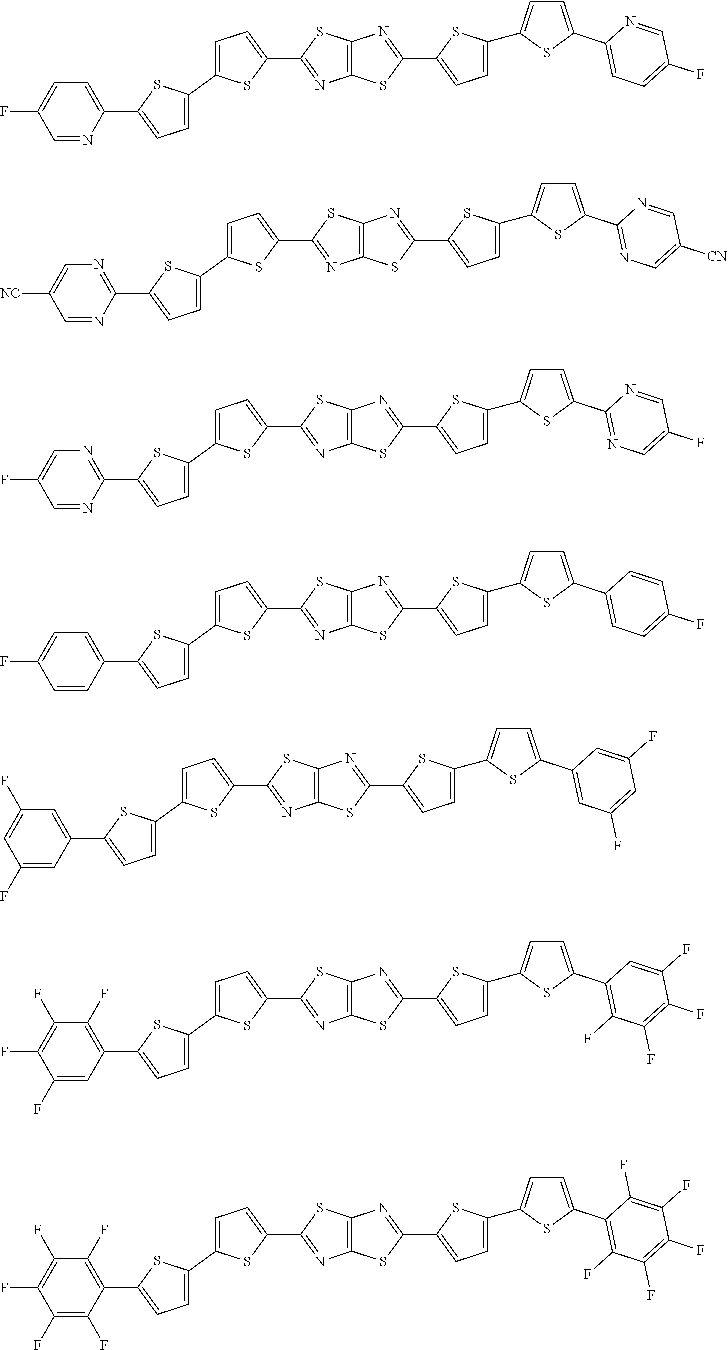

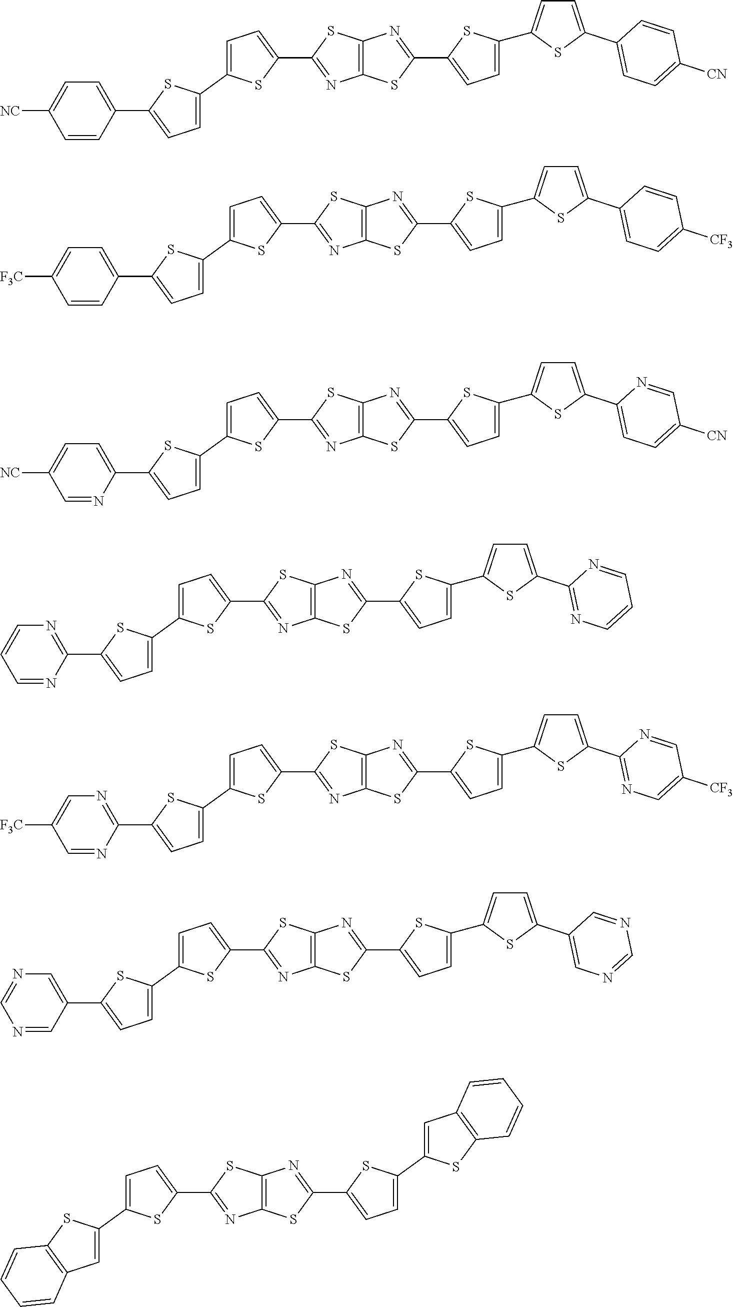

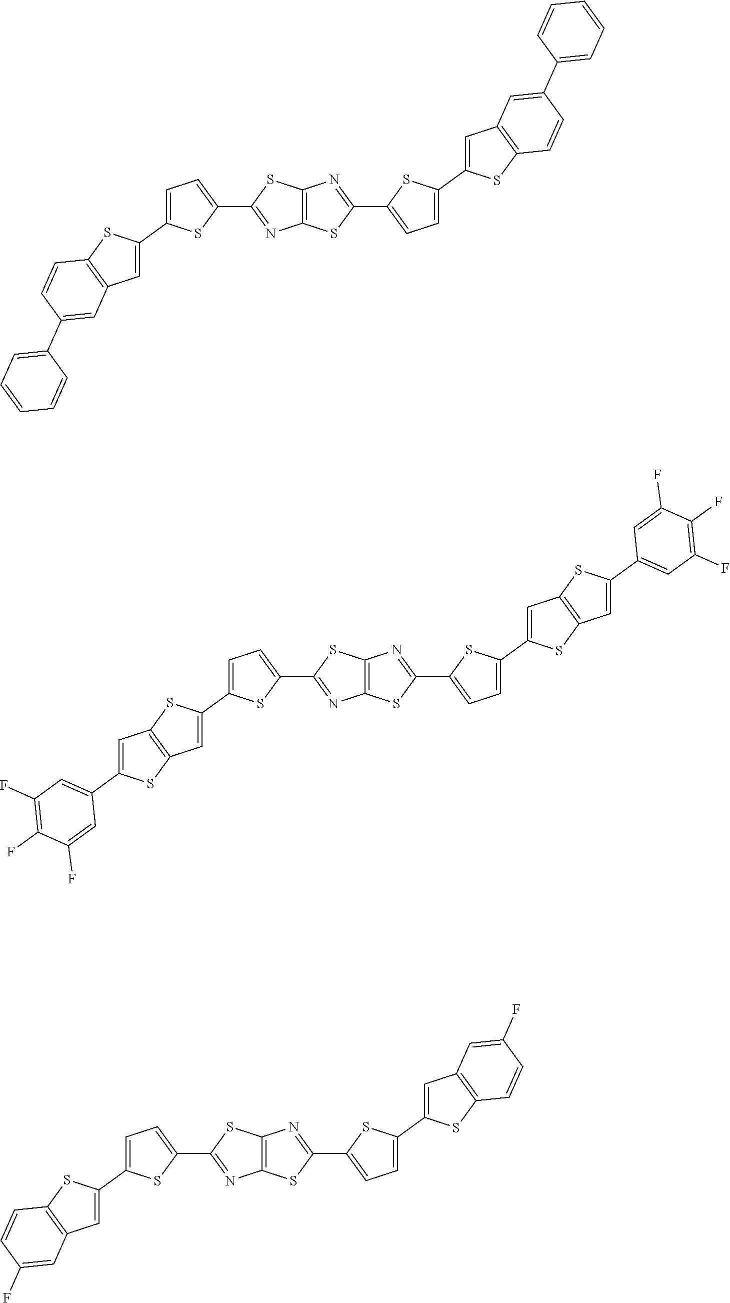 [SMILES: c1ccc(-c2ccc3sc(-c4ccc(-c5nc6sc(-c7ccc(-c8cc9cc(-c%10ccccc%10)ccc9s8)s7)nc6s5)s4)cc3c2)cc1 ; Fc1cc(-c2cc3sc(-c4ccc(-c5nc6sc(-c7ccc(-c8cc9sc(-c%10cc(F)c(F)c(F)c%10)cc9s8)s7)nc6s5)s4)cc3s2)cc(F)c1F ; Fc1ccc2sc(-c3ccc(-c4nc5sc(-c6ccc(-c7cc8cc(F)ccc8s7)s6)nc5s4)s3)cc2c1]

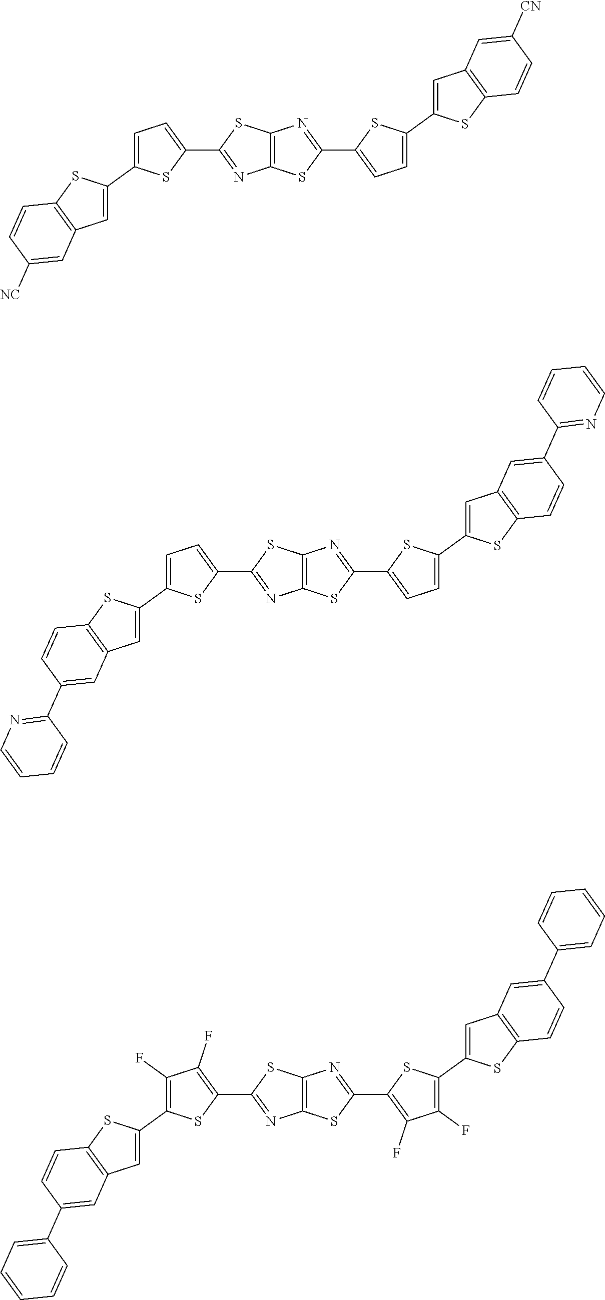 [SMILES: N#Cc1ccc2sc(-c3ccc(-c4nc5sc(-c6ccc(-c7cc8cc(C#N)ccc8s7)s6)nc5s4)s3)cc2c1 ; c1ccc(-c2ccc3sc(-c4ccc(-c5nc6sc(-c7ccc(-c8cc9cc(-c%10ccccn%10)ccc9s8)s7)nc6s5)s4)cc3c2)nc1 ; Fc1c(-c2cc3cc(-c4ccccc4)ccc3s2)sc(-c2nc3sc(-c4sc(-c5cc6cc(-c7ccccc7)ccc6s5)c(F)c4F)nc3s2)c1F]

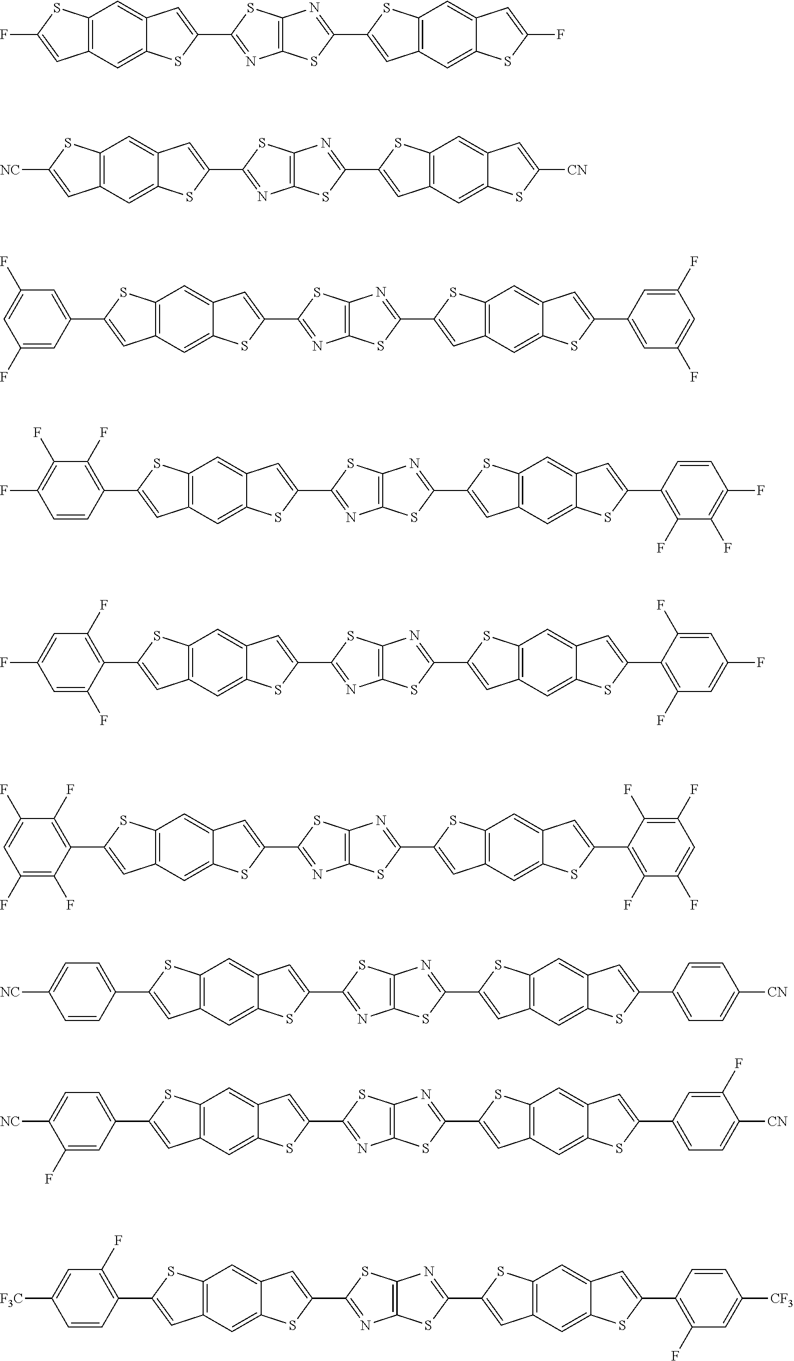

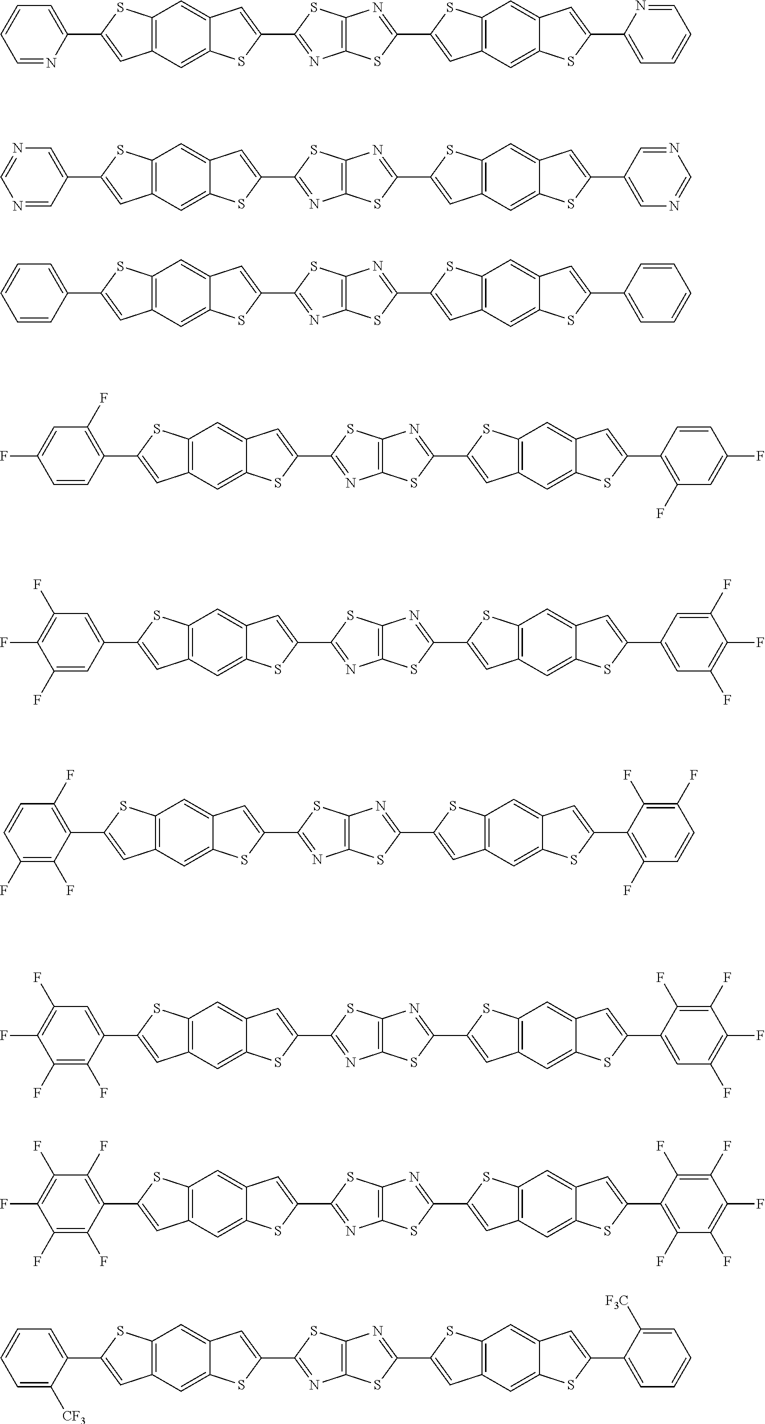

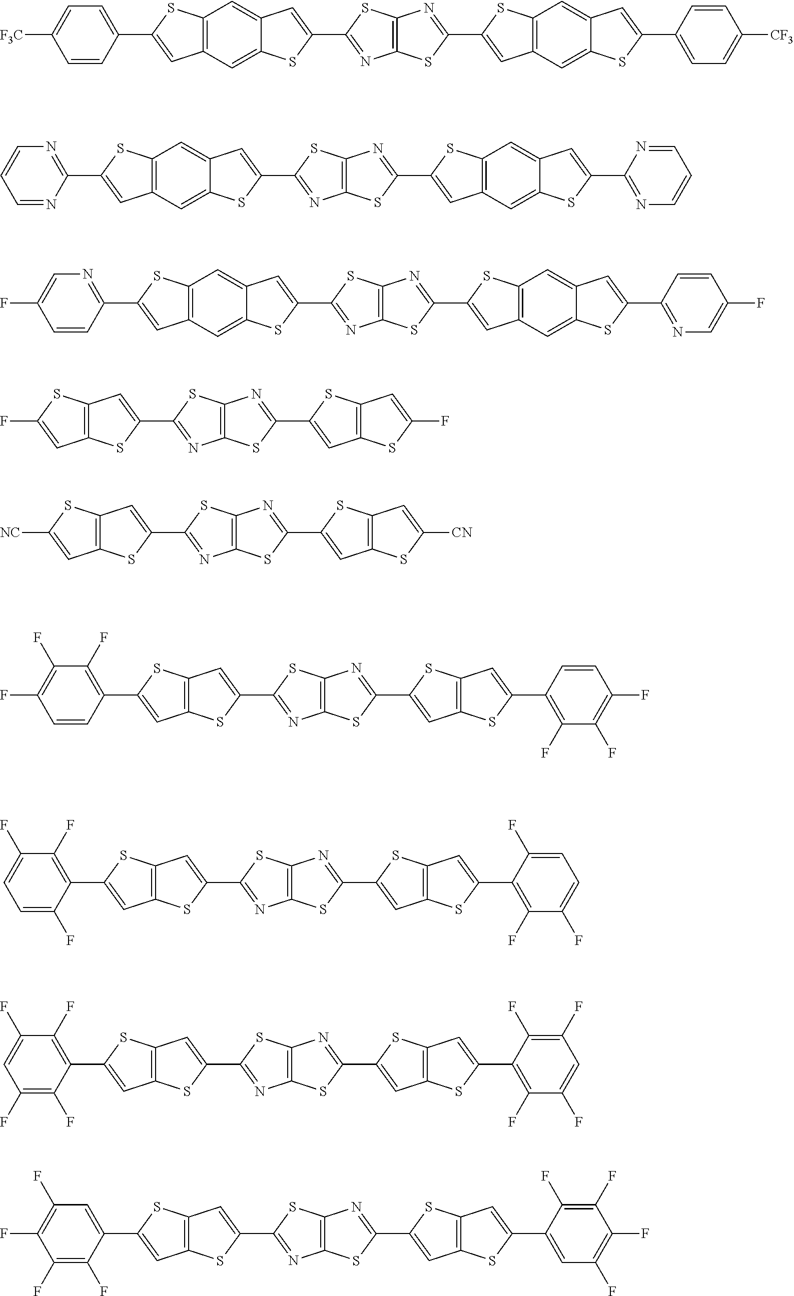

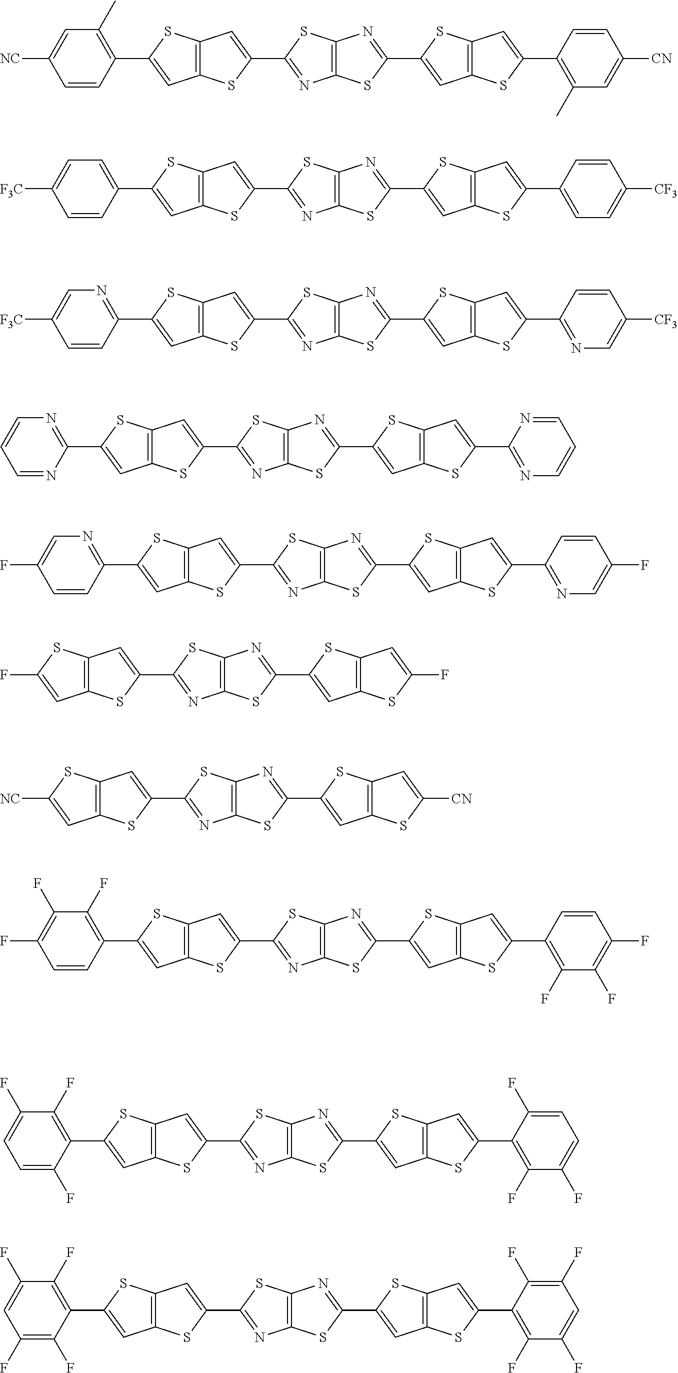

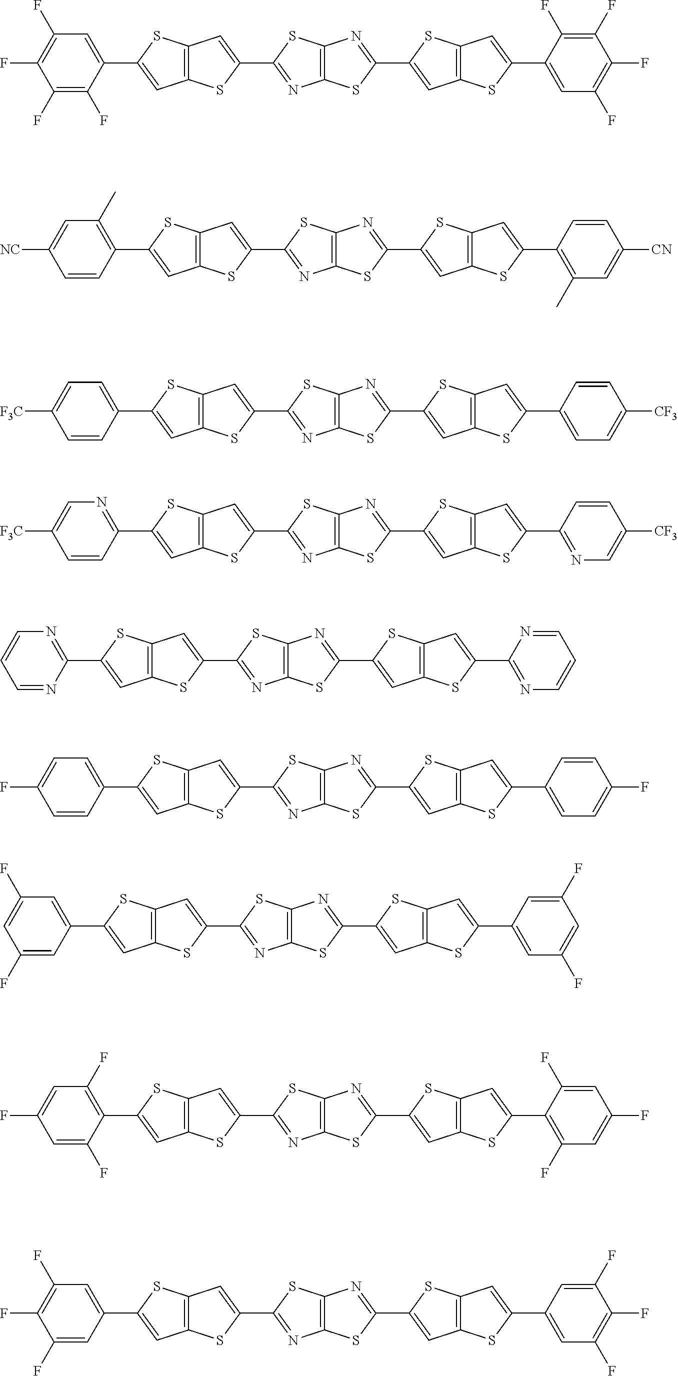

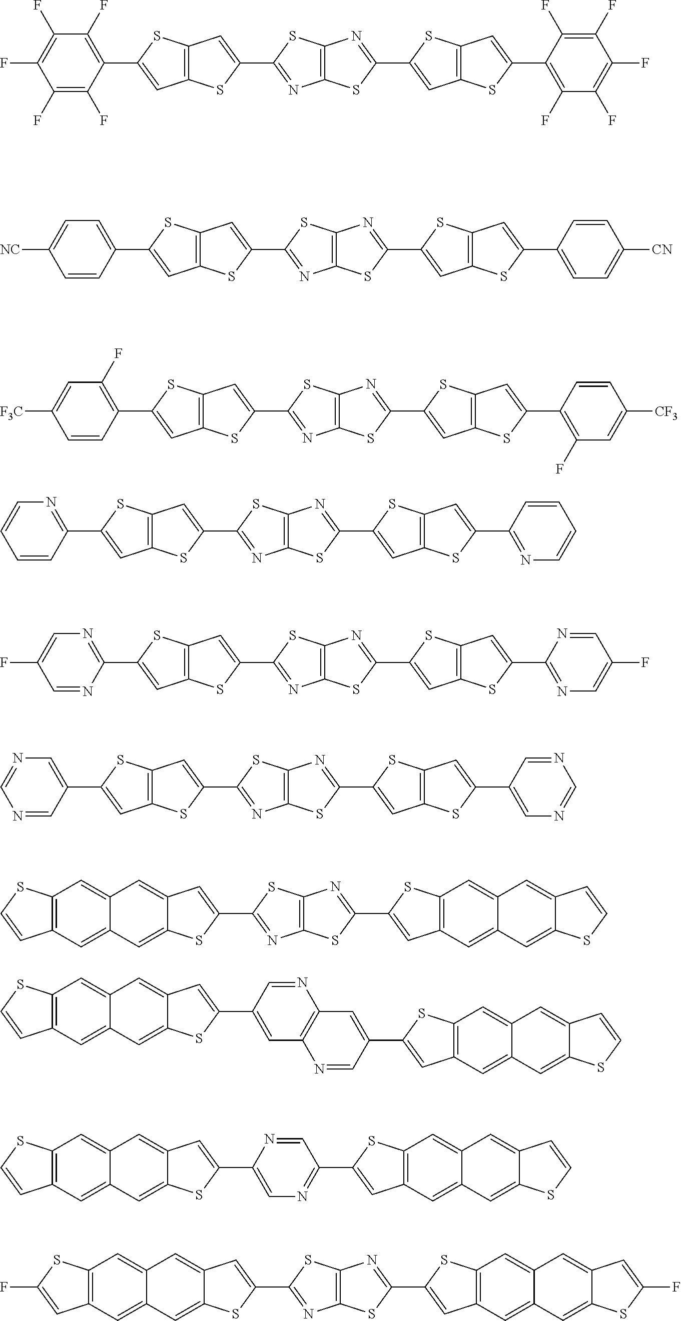

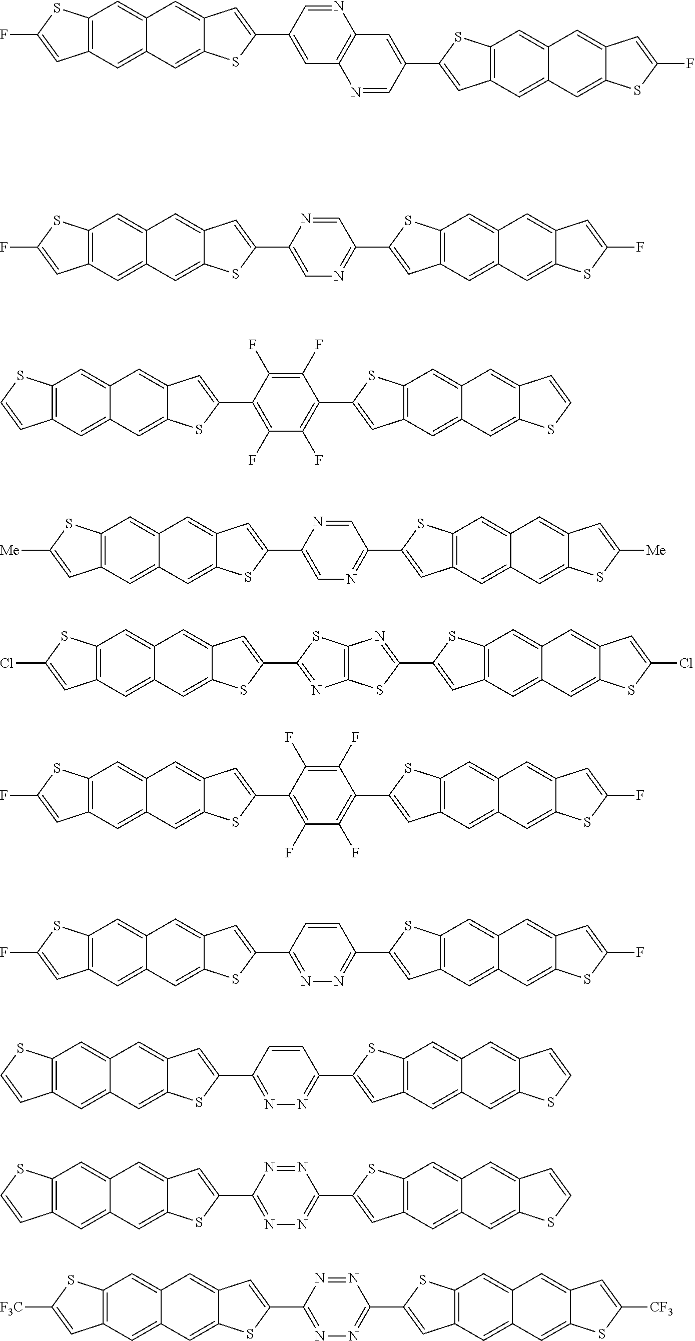

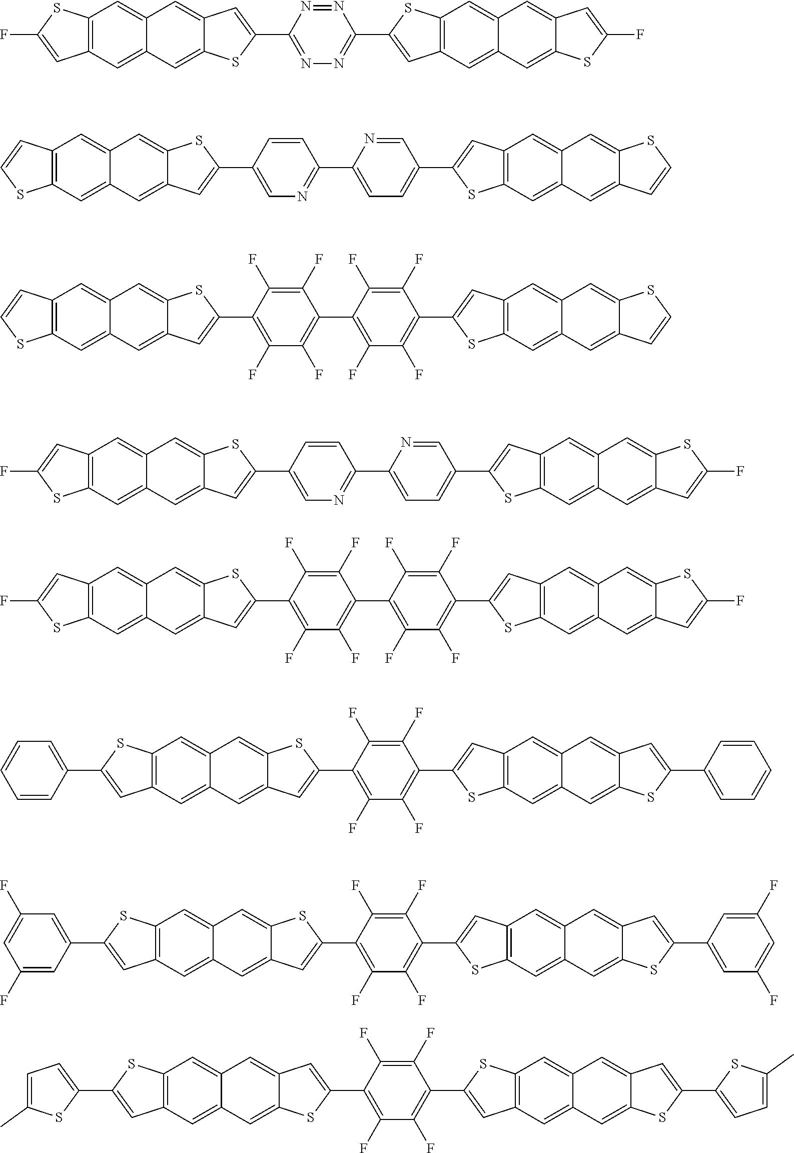

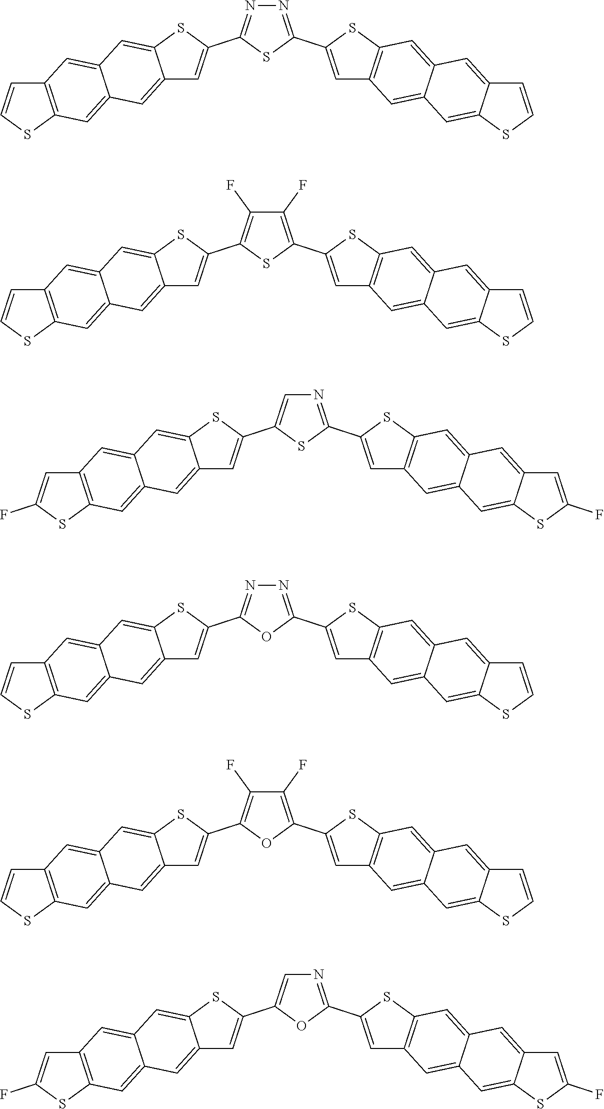 [SMILES: c1cc2cc3cc4sc(-c5nnc(-c6cc7cc8cc9sccc9cc8cc7s6)s5)cc4cc3cc2s1 ; Fc1c(-c2cc3cc4cc5sccc5cc4cc3s2)sc(-c2cc3cc4cc5sccc5cc4cc3s2)c1F ; Fc1cc2cc3cc4sc(-c5cnc(-c6cc7cc8cc9sc(F)cc9cc8cc7s6)s5)cc4cc3cc2s1 ; c1cc2cc3cc4sc(-c5nnc(-c6cc7cc8cc9sccc9cc8cc7s6)o5)cc4cc3cc2s1 ; Fc1c(-c2cc3cc4cc5sccc5cc4cc3s2)oc(-c2cc3cc4cc5sccc5cc4cc3s2)c1F ; Fc1cc2cc3cc4sc(-c5cnc(-c6cc7cc8cc9sc(F)cc9cc8cc7s6)o5)cc4cc3cc2s1]

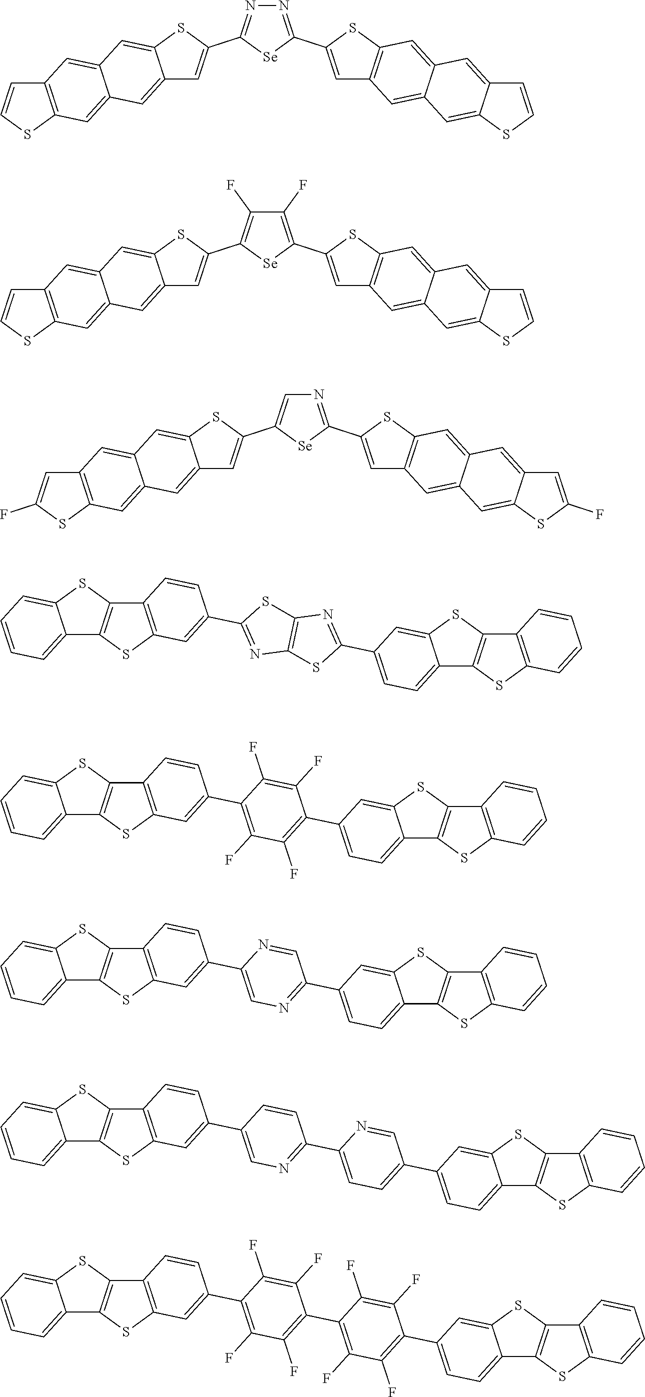

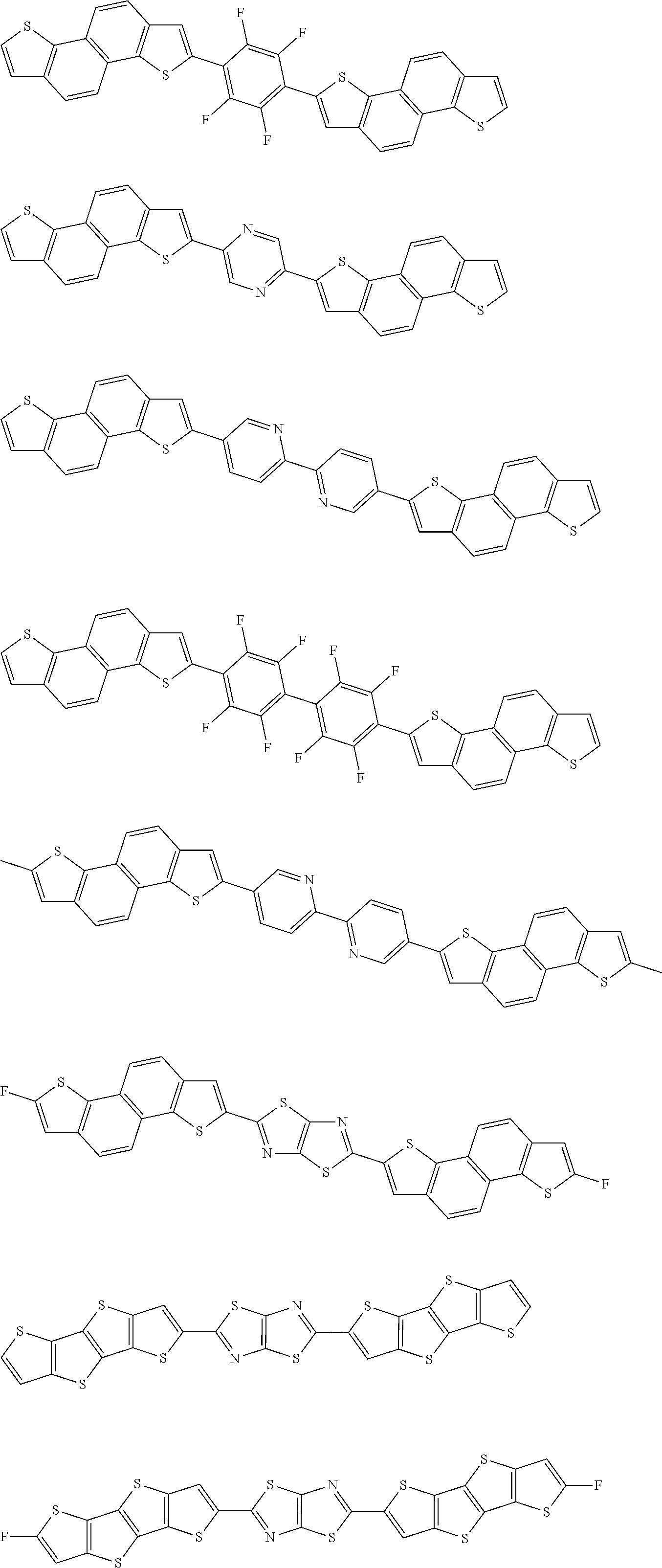

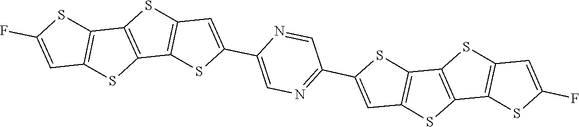 [SMILES: Fc1cc2sc3c4sc(-c5cnc(-c6cc7sc8c9sc(F)cc9sc8c7s6)cn5)cc4sc3c2s1]

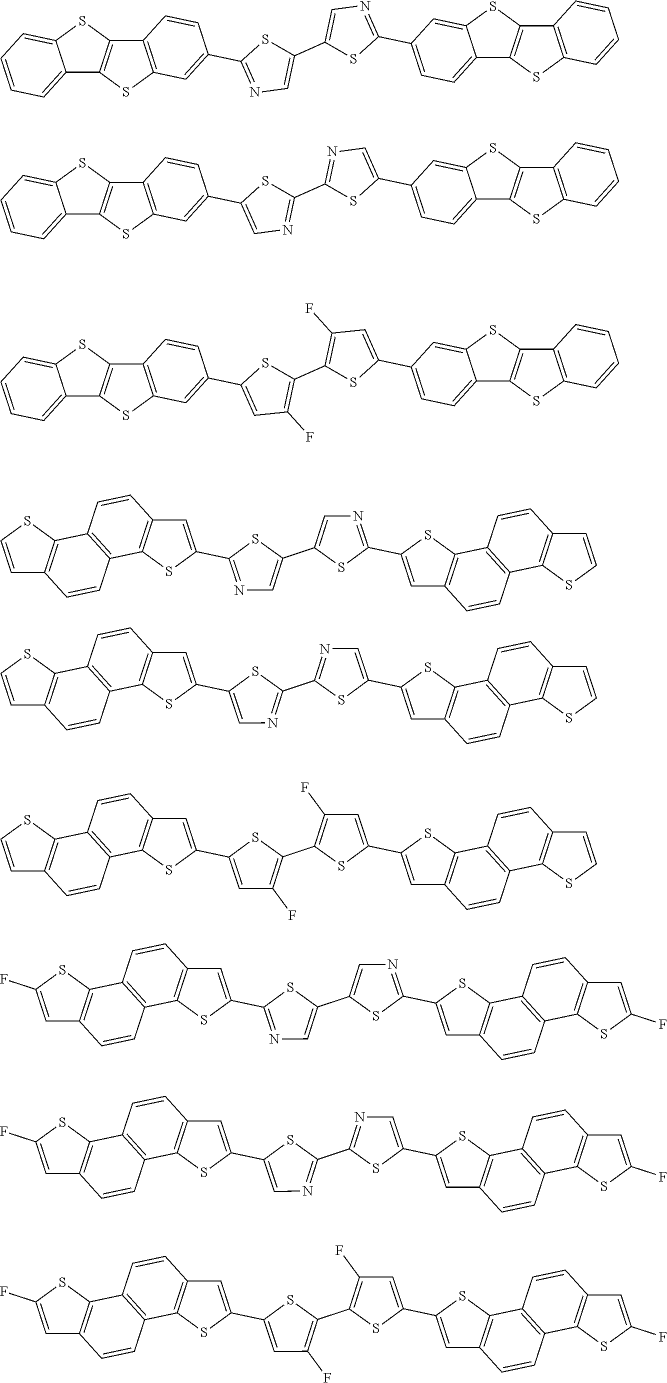

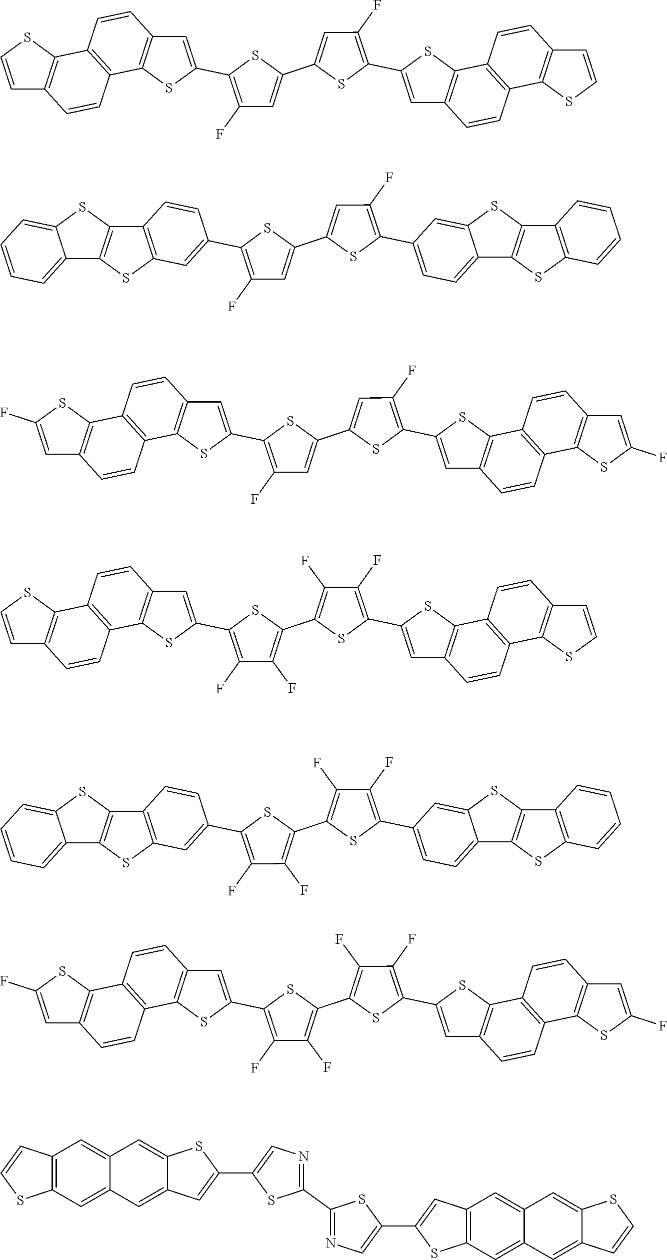

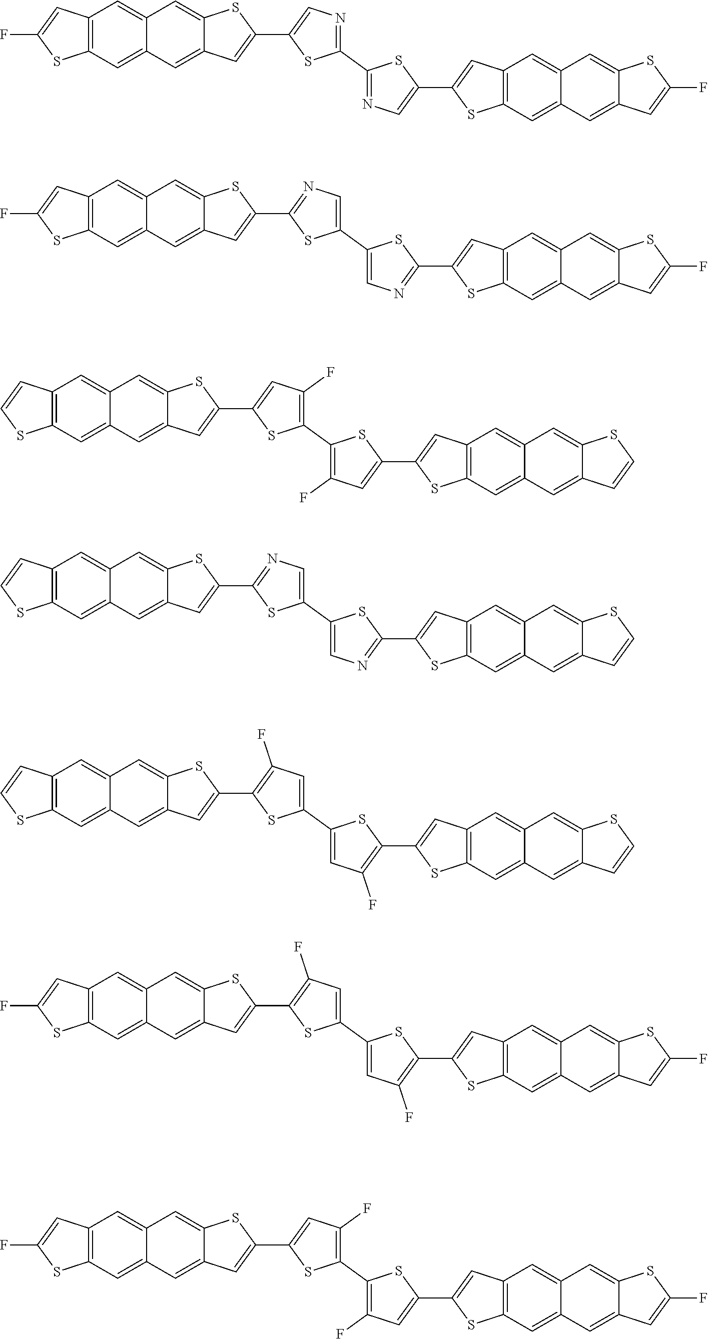

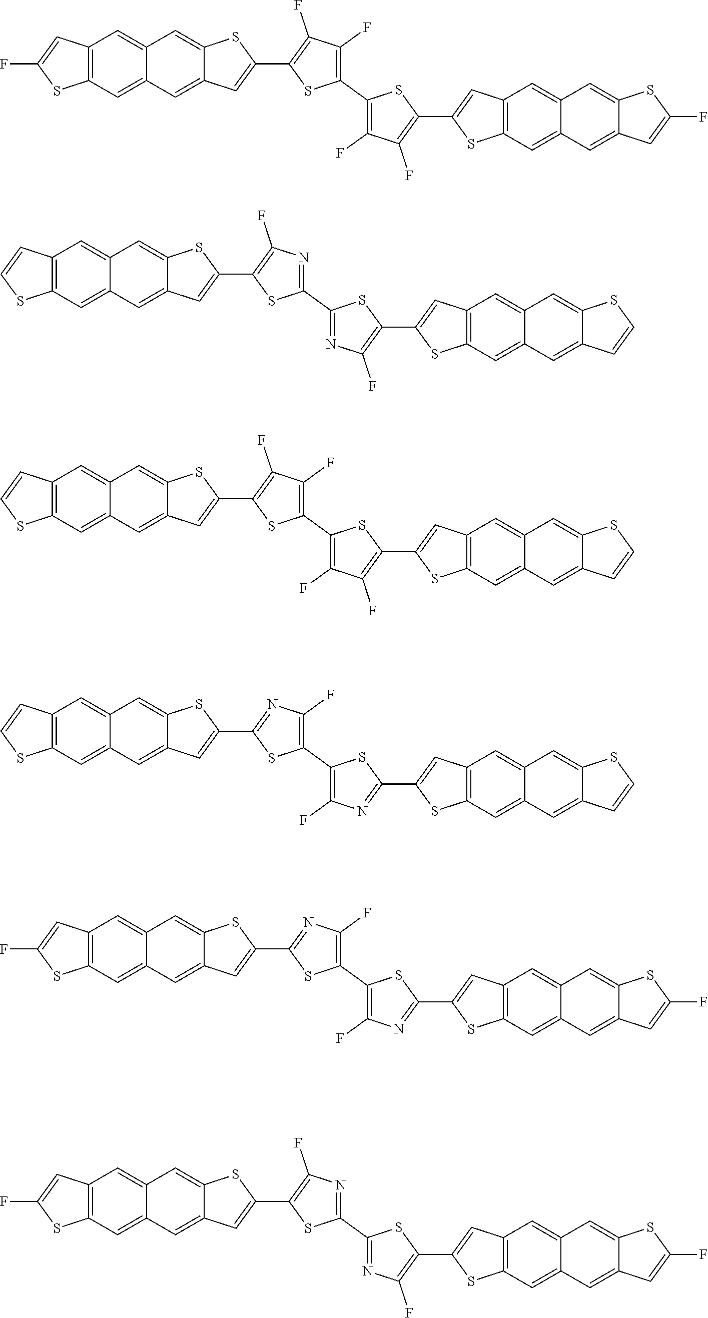 [SMILES: Fc1cc2cc3cc4sc(-c5sc(-c6sc(-c7cc8cc9cc%10sc(F)cc%10cc9cc8s7)c(F)c6F)c(F)c5F)cc4cc3cc2s1 ; Fc1nc(-c2nc(F)c(-c3cc4cc5cc6sccc6cc5cc4s3)s2)sc1-c1cc2cc3cc4sccc4cc3cc2s1 ; Fc1c(-c2cc3cc4cc5sccc5cc4cc3s2)sc(-c2sc(-c3cc4cc5cc6sccc6cc5cc4s3)c(F)c2F)c1F ; Fc1nc(-c2cc3cc4cc5sccc5cc4cc3s2)sc1-c1sc(-c2cc3cc4cc5sccc5cc4cc3s2)nc1F ; Fc1cc2cc3cc4sc(-c5nc(F)c(-c6sc(-c7cc8cc9cc%10sc(F)cc%10cc9cc8s7)nc6F)s5)cc4cc3cc2s1 ; Fc1cc2cc3cc4sc(-c5sc(-c6nc(F)c(-c7cc8cc9cc%10sc(F)cc%10cc9cc8s7)s6)nc5F)cc4cc3cc2s1]

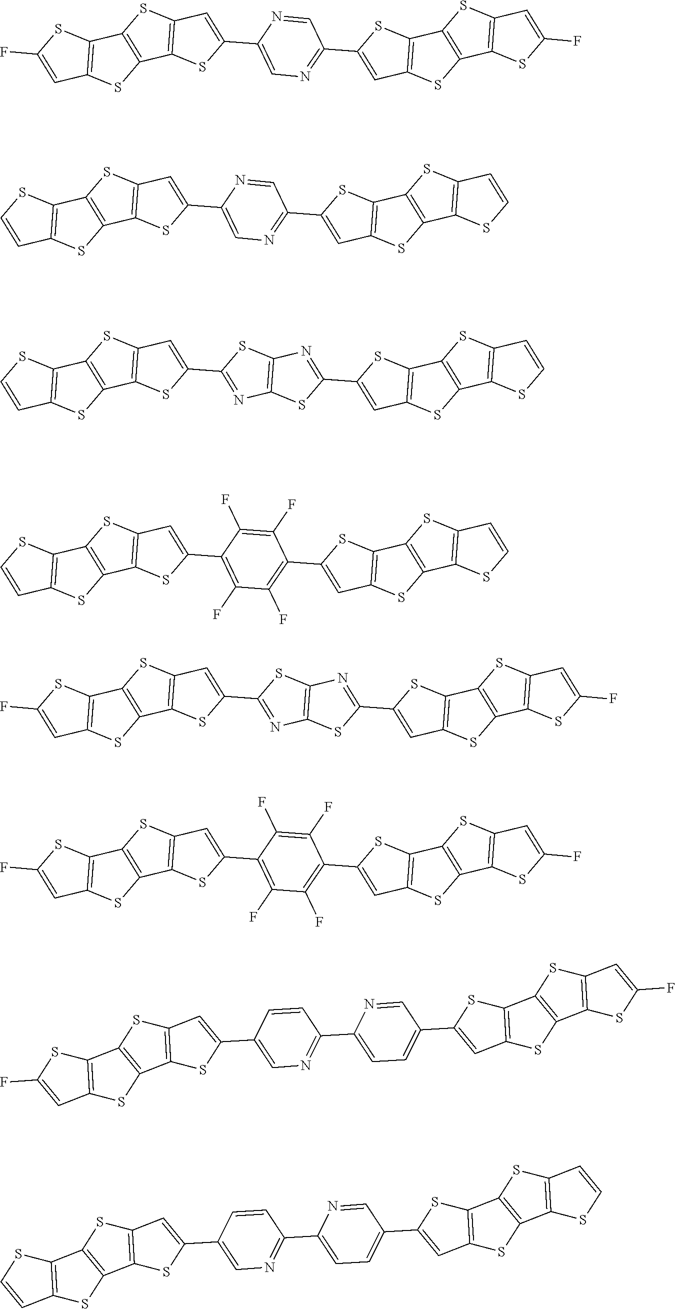

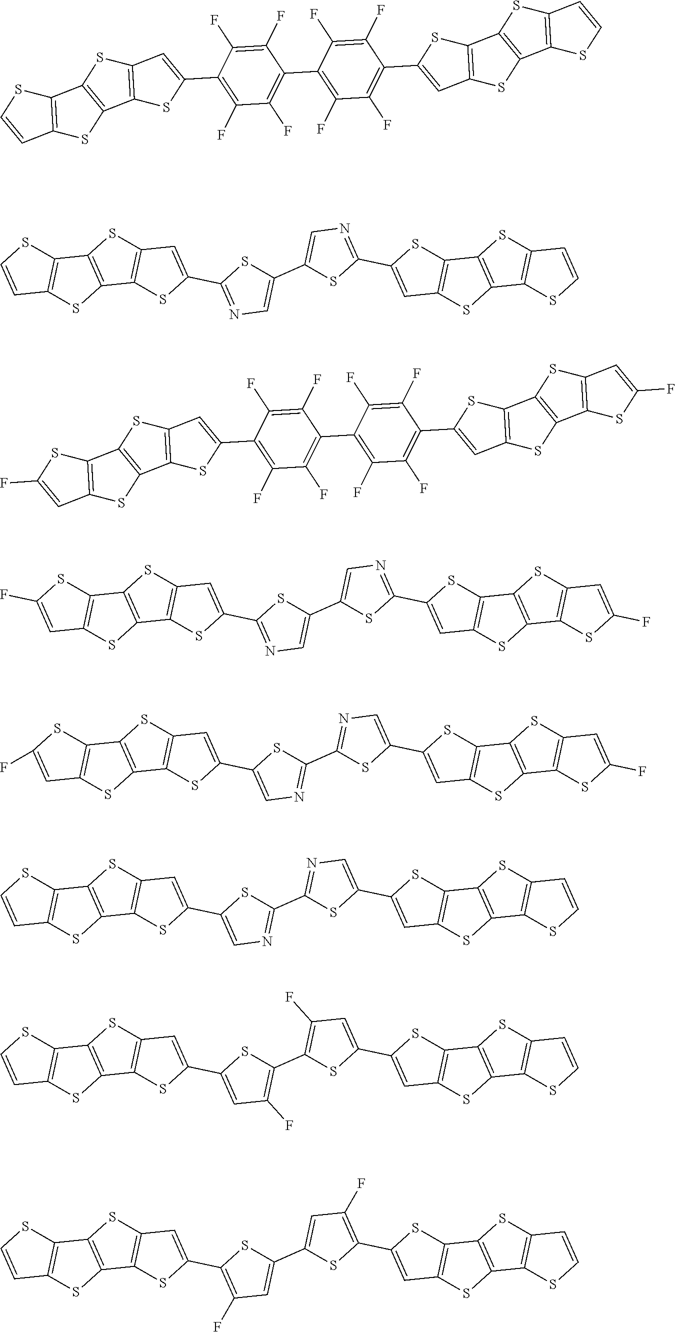

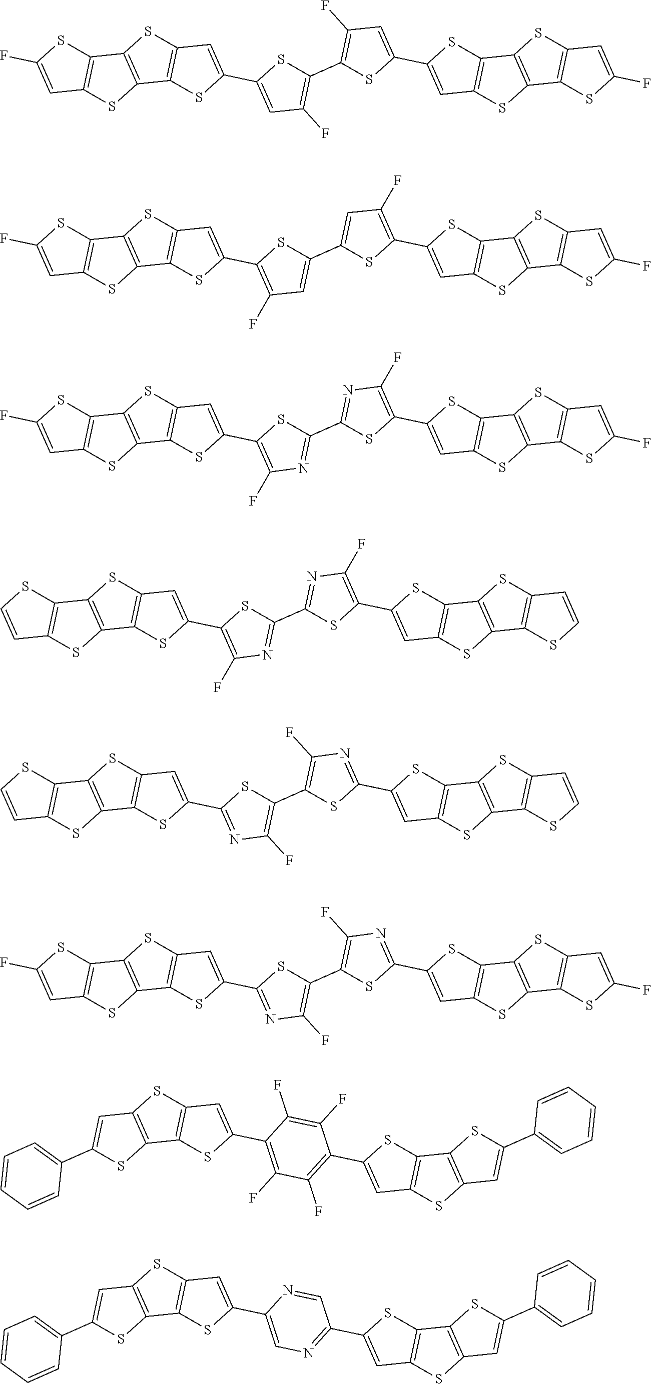

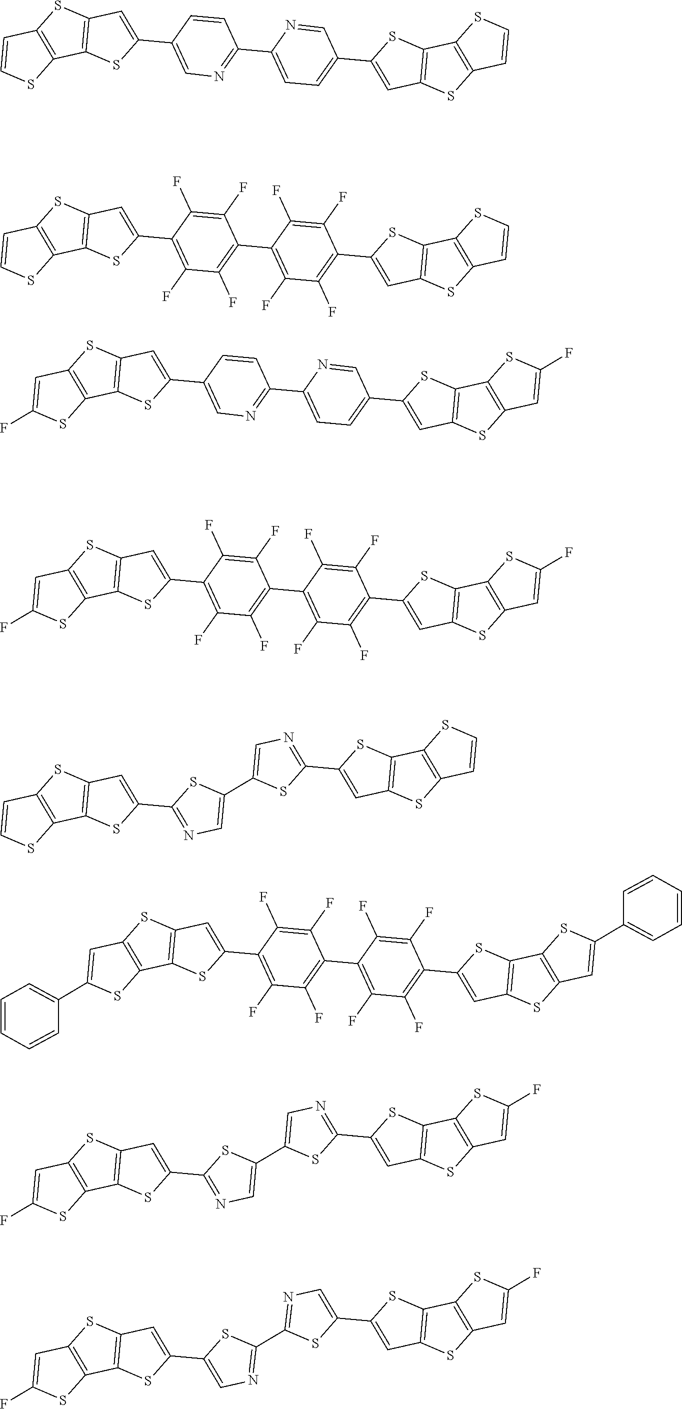

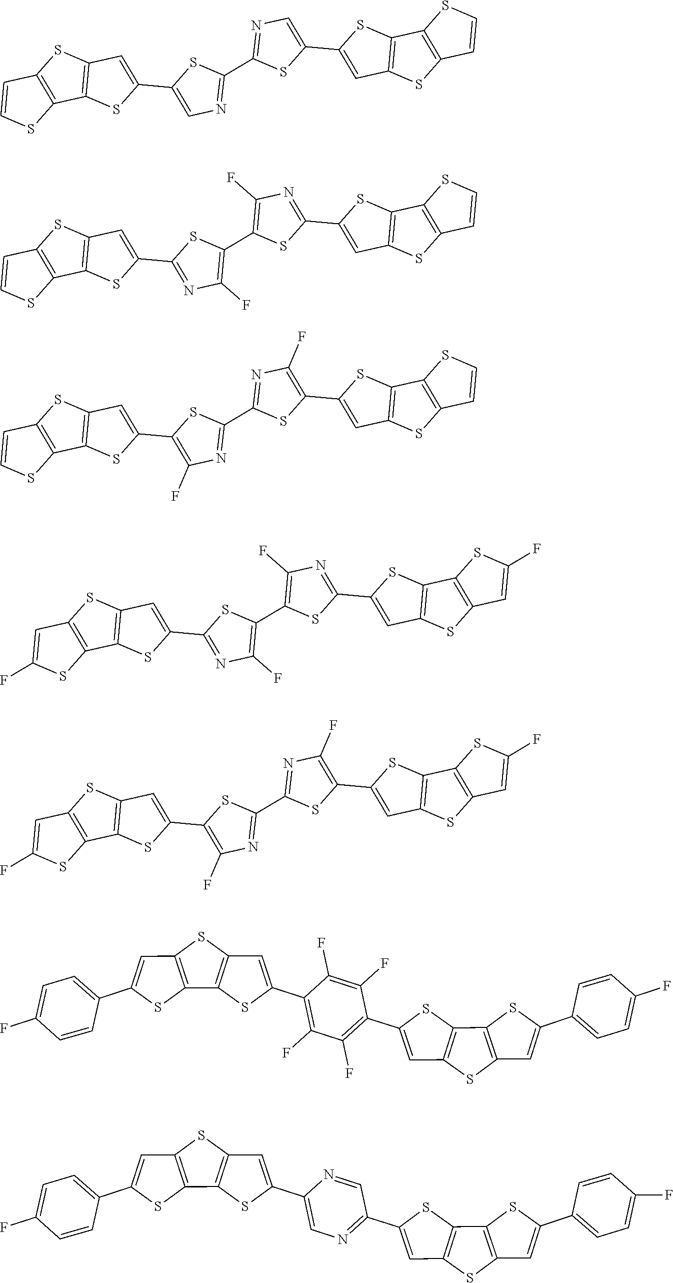

A molecular weight of the specific compound is not particularly limited, but is preferably 400 to 1200, and more preferably 400 to 900. In a case where the molecular weight is 1200 or less, a vapor deposition temperature is not increased, and the compound is not easily decomposed. In a case where the molecular weight is 400 or more, a glass transition point of a vapor deposition film is not lowered, and the heat resistance of the photoelectric conversion element is improved.

The specific compound is particularly useful as a material of the photoelectric conversion film used for the imaging element, the optical sensor, or a photoelectric cell. The specific compound can also be used as a coloring material, a liquid crystal material, an organic semiconductor material, a charge transport material, a pharmaceutical material, and a fluorescent diagnostic material.

The specific compound is preferably a compound in which an ionization potential in a single film is −5.0 to −6.0 eV from the viewpoints of matching of energy levels between the compound and the n-type semiconductor material described later.

The maximum absorption wavelength of the specific compound is not particularly limited and is, for example, preferably within a range of 350 to 550 nm and more preferably within a range of 400 to 550 nm.

The maximum absorption wavelength is a value measured in a solution state (solvent: chloroform) by an absorption spectrum of the specific compound being adjusted to a concentration having an absorbance of about 0.5 to 1. However, in a case where the specific compound is not soluble in chloroform, a value measured by using the specific compound in which the specific compound is vapor-deposited and formed into a film state is defined as a maximum absorption wavelength of the specific compound.

The maximum absorption wavelength of the photoelectric conversion film is not particularly limited and is, for example, preferably within a range of 300 to 700 nm and more preferably within a range of 400 to 700 nm.

From the viewpoint of the responsiveness of the photoelectric conversion element, a content of the specific compound in the photoelectric conversion film (=(film thickness of specific compound in terms of single layer/film thickness of photoelectric conversion film)×100) is preferably 15% to 75% by volume, more preferably 20% to 60% by volume, and still more preferably 25% to 40% by volume.

The specific compound may be used alone, or two or more thereof may be used in combination.

<Coloring Agent>

The photoelectric conversion film contains a coloring agent as another component in addition to the specific compound described above.

The coloring agent is preferably an organic coloring agent.

Examples of the coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, a quinoxaline coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, a metal complex coloring agent, compounds disclosed in paragraphs [0083] to [0089] of JP2014-82483A, compounds disclosed in paragraphs [0029] to [0033] of JP2009-167348A, compounds disclosed in paragraphs [0197] to [0227] of JP2012-77064A, compounds disclosed in paragraphs [0035] to [0038] of WO2018-105269A, compounds disclosed in paragraphs [0041] to [0043] of WO2018-186389A, compounds disclosed in paragraphs [0059] to [0062] of WO2018-186397A, compounds disclosed in paragraphs [0078] to [0083] of WO2019-009249A, compounds disclosed in paragraphs [0054] to [0056] of WO2019-049946A, compounds disclosed in paragraphs [0059] to [0063] of WO2019-054327A, compounds disclosed in paragraphs [0086] to [0087] of WO2019-098161A, and compounds disclosed in paragraphs [0085] to [0114] of WO2020-013246.

A content of the coloring agent with respect to the total content of the specific compound and the coloring agent in the photoelectric conversion film (=(film thickness of coloring agent in terms of single layer/(film thickness of specific compound in terms of single layer+film thickness of coloring agent in terms of single layer)×100)) is preferably 15% to 75% by volume, more preferably 20% to 60% by volume, and still more preferably 25% to 50% by volume.

The coloring agent may be used alone, or two or more thereof may be used in combination.

<n-Type Semiconductor Material>

The photoelectric conversion film preferably further includes the n-type semiconductor material as another component in addition to the specific compound and coloring agent described above.

The n-type semiconductor material is an acceptor-property organic semiconductor material (a compound), and refers to an organic compound having a property of easily accepting an electron.

More specifically, the n-type semiconductor material is preferably an organic compound having a higher electron affinity than that of the specific compound in a case where the n-type semiconductor material is used by being brought in contact with the above-described specific compound.

In the present specification, a value (value multiplied by −1) of a reciprocal number of the LUMO value obtained by the calculation of B3LYP/6-31G (d) using Gaussian '09 (software manufactured by Gaussian, Inc.) as a value of the electron affinity.

In addition, the n-type semiconductor material is preferably an organic compound having a higher electron affinity than the coloring agent in a case where the n-type semiconductor material is used by being brought in contact with the above-described coloring agent.

The electron affinity of the n-type semiconductor material is preferably 3.0 to 5.0 eV.

Examples of the n-type semiconductor material include fullerenes selected from the group consisting of a fullerene and derivatives thereof, fused aromatic carbocyclic compounds (for example, a naphthalene derivative, an anthracene derivative, a phenanthrene derivative, a tetracene derivative, a pyrene derivative, a perylene derivative, and a fluoranthene derivative); a heterocyclic compound having a 5- to 7-membered ring having at least one of a nitrogen atom, an oxygen atom, or a sulfur atom (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, and thiazole); polyarylene compounds; fluorene compounds; cyclopentadiene compounds; silyl compounds; 1,4,5,8-naphthalenetetracarboxylic acid anhydride; 1,4,5,8-naphthalenetetracarboxylic acid anhydride imide derivative; oxadiazole derivative; anthraquinodimethane derivatives; diphenylquinone derivatives; bathocuproine, bathophenanthroline, and derivatives thereof; triazole compounds; a distyrylarylene derivative; a metal complex having a nitrogen-containing heterocyclic compound as a ligand; a silole compound; and compounds disclosed in paragraphs [0056] to [0057] of JP2006-100767A.

Among these, it is preferable that examples of the n-type semiconductor material include fullerenes selected from the group consisting of a fullerene and derivatives thereof.

Examples of the fullerenes include a fullerene C₆₀, a fullerene C₇₀, a fullerene C₇₆, a fullerene C₇₈, a fullerene C₈₀, a fullerene C₈₂, a fullerene C₈₄, a fullerene C₉₀, a fullerene C₉₆, a fullerene C₂₄₀, a fullerene C₅₄₀, and a mixed fullerene.

Examples of the fullerene derivatives include compounds in which a substituent is added to the above fullerenes. The substituent is preferably an alkyl group, an aryl group, or a heterocyclic group. The fullerene derivative is preferably compounds described in JP2007-123707A.

In a case where the photoelectric conversion film includes the n-type semiconductor material, a content of the n-type semiconductor material with respect to the total content of the specific compound, the coloring agent, and the n-type semiconductor material (=(film thicknesses of n-type semiconductor material in terms of single layer/(film thickness of specific compound in terms of single layer+film thicknesses of coloring agent in terms of single layer+film thickness of n-type semiconductor material in terms of single layer)×100)) is preferably 15% to 75% by volume, more preferably 20% to 60% by volume, and still more preferably 25% to 50% by volume.

The n-type semiconductor material may be used alone, or two or more thereof may be used in combination.

In addition, in a case where the n-type semiconductor material includes fullerenes, a content of the fullerenes to a total content of the n-type semiconductor material (=(film thickness of fullerenes in terms of single layer/total film thickness of n-type semiconductor materials in terms of single layer)×100) is preferably 50% to 100% by volume, and more preferably 80% to 100% by volume.

The fullerenes may be used alone, or two or more thereof may be used in combination.

The molecular weight of the n-type semiconductor material is preferably 200 to 1200, and more preferably 200 to 1000.

The photoelectric conversion film is substantially preferably composed of the specific compound, the coloring agent, and the n-type semiconductor material. “The photoelectric conversion film is substantially composed of only the specific compound, the coloring agent, and the n-type semiconductor material” means “the total content of the specific compound, the coloring agent, and the n-type semiconductor material with respect to the total mass of the photoelectric conversion film is 95% to 100% by mass”.

The photoelectric conversion film is preferably a mixture layer formed in a state where the specific compound and the coloring agent are mixed.

In addition, in a case where the photoelectric conversion film contains an n-type semiconductor material, the photoelectric conversion film is preferably a mixture layer formed in a state where the specific compound, and the n-type semiconductor material are mixed.

In a case where the photoelectric conversion film contains a coloring agent and an n-type semiconductor material, the photoelectric conversion film is preferably a mixture layer formed in a state where the specific compound, the coloring agent, and the n-type semiconductor material are mixed.

The mixture layer is a layer in which two or more materials are mixed in a single layer.

The photoelectric conversion film containing the specific compound is a non-light emitting film, and has a feature different from organic light emitting diodes (OLEDs). The non-light emitting film is intended for a film having a light emission quantum efficiency of 1% or less, and the light emission quantum efficiency is preferably 0.5% or less, and more preferably 0.1% or less.

<Film Formation Method>

The photoelectric conversion film can be formed mostly by a dry film formation method. Examples of the dry film formation method include a physical vapor deposition method such as a vapor deposition method (in particular, a vacuum vapor deposition method), a sputtering method, and an ion plating method, a molecular beam epitaxy (MBE) method, and a chemical vapor deposition (CVD) method such as plasma polymerization. Among these, the vacuum vapor deposition method is preferable. In a case where the photoelectric conversion film is formed by the vacuum vapor deposition method, manufacturing conditions such as a degree of vacuum and a vapor deposition temperature can be set according to the normal method.

The thickness of the photoelectric conversion film is preferably 10 to 1000 nm, more preferably 50 to 800 nm, still more preferably 50 to 500 nm, and particularly preferably 50 to 400 nm.

<Electrode>

Electrodes (the upper electrode (the transparent conductive film) 15 and the lower electrode (the conductive film) 11) are formed of conductive materials. Examples of the conductive material include metals, alloys, metal oxides, electrically conductive compounds, and mixtures thereof.

Since light is incident through the upper electrode 15, the upper electrode 15 is preferably transparent to light to be detected. Examples of the materials constituting the upper electrode 15 include conductive metal oxides such as tin oxide (antimony tin oxide (ATO), fluorine doped tin oxide (FTO)) doped with antimony, fluorine, or the like, tin oxide, zinc oxide, indium oxide, indium tin oxide (ITO), and indium zinc oxide (IZO); metal thin films such as gold, silver, chromium, and nickel; mixtures or laminates of these metals and the conductive metal oxides; organic conductive materials such as polyaniline, polythiophene, and polypyrrole; carbon materials such as graphene and carbon nanotubes. Among these, conductive metal oxides are preferable from the viewpoints of high conductivity, transparency, and the like.

In general, in a case where the conductive film is made to be thinner than a certain range, a resistance value is rapidly increased. However, in the solid-state imaging element into which the photoelectric conversion element according to the present embodiment is incorporated, the sheet resistance is, for example, 100 to 10000Ω/□, and a degree of freedom of a range of the film thickness that can be thinned is large. In addition, as the thickness of the upper electrode (the transparent conductive film) 15 is thinner, the amount of light that the upper electrode absorbs is smaller, and the light transmittance usually increases. The increase in the light transmittance causes an increase in light absorbance in the photoelectric conversion film and an increase in the photoelectric conversion ability, which is preferable. Considering the suppression of leakage current, an increase in the resistance value of the thin film, and an increase in transmittance accompanied by the thinning, the film thickness of the upper electrode 15 is preferably 5 to 100 nm, and more preferably 5 to 20 nm.

There is a case where the lower electrode 11 has transparency or an opposite case where the lower electrode 11 does not have transparency and reflects light, depending on the application. Examples of a material constituting the lower electrode 11 include conductive metal oxides such as tin oxide (ATO, FTO) doped with antimony, fluorine, or the like, tin oxide, zinc oxide, indium oxide, indium tin oxide (ITO), and indium zinc oxide (IZO); metals such as gold, silver, chromium, nickel, titanium, tungsten, and aluminum, and conductive compounds (for example, titanium nitride (TiN)) such as oxides or nitrides of these metals; mixtures or laminates of these metals and conductive metal oxides; organic conductive materials such as polyaniline, polythiophene, and polypyrrole; carbon materials such as graphene and carbon nanotubes.

The method of forming electrodes is not particularly limited, and can be appropriately selected in accordance with the electrode material. Specific examples thereof include a wet method such as a printing method and a coating method; a physical method such as a vacuum vapor deposition method, a sputtering method, and an ion plating method; and a chemical method such as a CVD method and a plasma CVD method.

In a case where the material of the electrode is ITO, examples thereof include an electron beam method, a sputtering method, a resistance heating vapor deposition method, a chemical reaction method (such as a sol-gel method), and a coating method with a dispersion of indium tin oxide.

<Charge Blocking Film: Electron Blocking Film and Positive Hole Blocking Film>

It is also preferable that the photoelectric conversion element according to the embodiment of the present invention has one or more interlayers between the conductive film and the transparent conductive film, in addition to the photoelectric conversion film. An example of the interlayer includes a charge blocking film. In a case where the photoelectric conversion element has this film, the characteristics (such as photoelectric conversion efficiency and responsiveness) of the obtained photoelectric conversion element are more excellent. Examples of the charge blocking film include an electron blocking film and a positive hole blocking film. Hereinafter, each of the films will be described in detail.

(Electron Blocking Film)

The electron blocking film is a donor organic semiconductor material (compound), and a p-type organic semiconductor described below can be used, for example. The p-type organic semiconductor may be used alone, or two or more thereof may be used in combination.

Examples of the p-type organic semiconductor include triarylamine compounds (for example, —N, N′-bis (3-methylphenyl)-(1,1′-biphenyl)-4,4′-diamine (TPD), 4,4′-bis [N-(naphthyl)-N-Phenyl-amino] biphenyl (α-NPD), compounds disclosed in paragraphs [0128] to [0148] of JP2011-228614A, compounds disclosed in paragraphs [0052] to [0063] of JP2011-176259A, compounds disclosed in paragraphs [0119] to [0158] of JP2011-225544A, compounds disclosed in paragraphs [0044] to [0051] of JP2015-153910A, and compounds disclosed in paragraphs [0086] to [0090] of JP2012-94660A, pyrazoline compounds, styrylamine compounds, hydrazone compounds, polysilane compounds, thiophene compounds (for example, a thienothiophene derivative, a dibenzothiophene derivative, a benzodithiophene derivative, a dithienothiophene derivative, a [1] benzothieno [3,2-b] thiophene (BTBT) derivative, a thieno [3,2-f: 4,5-f′] bis [1] benzothiophene (TBBT) derivative, compounds disclosed in paragraphs [0031] to [0036] of JP2018-014474A, compounds disclosed in paragraphs [0043] to [0045] of WO2016-194630A, compounds disclosed in paragraphs [0025] to [0037], and [0099] to [0109] of WO2017-159684A, compounds disclosed in paragraphs [0029] to [0034] of JP2017-076766A, compounds disclosed in paragraphs [0015] to [0025] of WO2018-207722A, compounds disclosed in paragraphs [0045] to [0053] of JP2019-054228A, compounds disclosed in paragraphs [0045] to [0055] of WO2019-058995A, compounds disclosed in paragraphs [0063] to [0089] of WO2019-081416A, compounds disclosed in paragraphs [0033] to [0036] of JP2019-080052A, compounds disclosed in paragraphs [0044] to [0054] of WO2019-054125A, compounds disclosed in paragraphs [0041] to [0046] of WO2019-093188A, and the like), a cyanine compound, an oxonol compound, a polyamine compound, an indole compound, a pyrrole compound, a pyrazole compound, a polyarylene compound, a fused aromatic carbocyclic compound (for example, a naphthalene derivative, an anthracene derivative, a phenanthrene derivative, a tetracene derivative, a pentacene derivative, a pyrene derivative, a perylene derivative, and a fluoranthene derivative), a porphyrin compound, a phthalocyanine compound, a triazole compound, an oxadiazole compound, an imidazole compound, a polyarylalkane compound, a pyrazolone compound, an amino-substituted chalcone compound, an oxazole compound, a fluorenone compound, a silazane compound, and a metal complex having nitrogen-containing heterocyclic compounds as ligands.

Examples of the p-type organic semiconductor include compounds having an ionization potential smaller than that of the n-type semiconductor material, and in a case where this condition is satisfied, the above-described coloring agents can be used.

A polymer material can also be used as the electron blocking film.

Examples of the polymer material include a polymer such as phenylenevinylene, fluorene, carbazole, indole, pyrene, pyrrole, picoline, thiophene, acetylene, and diacetylene, and a derivative thereof.

The electron blocking film may be formed of a plurality of films.

The electron blocking film may be formed of an inorganic material. In general, since an inorganic material has a dielectric constant larger than that of an organic material, in a case where the inorganic material is used in the electron blocking film, a large voltage is applied to the photoelectric conversion film. Therefore, the photoelectric conversion efficiency increases. Examples of the inorganic material that can be used for the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

(Positive Hole Blocking Film)

A positive hole blocking film is an acceptor-property organic semiconductor material (compound), and the n-type semiconductor material described above and the like can be used.

The method of manufacturing the charge blocking film is not particularly limited, and examples thereof include a dry film formation method and a wet film formation method. Examples of the dry film formation method include a vapor deposition method and a sputtering method. The vapor deposition method may be any of a physical vapor deposition (PVD) method and a chemical vapor deposition (CVD) method, and the physical vapor deposition method such as a vacuum vapor deposition method is preferable. Examples of the wet film formation method include an ink jet method, a spray method, a nozzle printing method, a spin coating method, a dip coating method, a casting method, a die coating method, a roll coating method, a bar coating method, and a gravure coating method, and an ink jet method is preferable from the viewpoint of high accuracy patterning.

Each thickness of the charge blocking films (the electron blocking film and the positive hole blocking film) is preferably 3 to 200 nm, more preferably 5 to 100 nm, and still more preferably 5 to 30 nm.

<Substrate>

The photoelectric conversion element may further include a substrate. Types of the substrate to be used are not particularly limited, and examples of the substrate include a semiconductor substrate, a glass substrate, and a plastic substrate.

A position of the substrate is not particularly limited, and in general, the conductive film, the photoelectric conversion film, and the transparent conductive film are laminated on the substrate in this order.

<Sealing Layer>

The photoelectric conversion element may further include a sealing layer. The performance of a photoelectric conversion material may deteriorate noticeably due to the presence of deterioration factors such as water molecules. The deterioration can be prevented by coating and sealing the entirety of the photoelectric conversion film with the sealing layer such as diamond-like carbon (DLC) or ceramics such as metal oxide, or metal nitride, and metal nitride oxide which are dense and into which water molecules do not permeate.

The material of the sealing layer may be selected and the sealing layer may be manufactured according to the description in paragraphs [0210] to [0215] of JP2011-082508A.

[Imaging Element and Optical Sensor]

An example of the application of the photoelectric conversion element includes an imaging element. The imaging element is an element that converts optical information of an image into an electric signal. In general, a plurality of the photoelectric conversion elements are arranged in a matrix on the same plane, and an optical signal is converted into an electric signal in each photoelectric conversion element (pixel) to sequentially output the electric signal to the outside of the imaging element for each pixel. Therefore, each pixel is formed of one or more photoelectric conversion elements and one or more transistors.

The imaging element is mounted on an imaging element such as a digital camera and a digital video camera, an electronic endoscope, and imaging modules such as a cellular phone.

The photoelectric conversion element according to the embodiment of the present invention is also preferably used for an optical sensor including the photoelectric conversion element of the present invention. The photoelectric conversion element may be used alone as the optical sensor, and the photoelectric conversion element may be used as a line sensor in which the photoelectric conversion elements are linearly arranged or as a two-dimensional sensor in which the photoelectric conversion elements are arranged in a plane shape.

[Compound]

The present invention also relates to a compound.

The compound according to the embodiment of the present invention is the same compound as the compound represented by Formula (2-2), the compound represented by Formula (3-2), the compound represented by Formula (3-3), the compound represented by Formula (3-4), the compound represented by Formula (3-5), the compound represented by Formula (3-6), the compound represented by Formula (1-4), the compound represented by Formula (1-5), the compound represented by Formula (1-6), the compound represented by Formula (1-7), and/or the compound represented by Formula (1-8) among the specific compounds described above, and the preferred conditions are also the same.

### Examples

The present invention will be described in more detail based on Examples below. Materials, used amounts, ratios, treatment contents, treatment procedures, and the like described in the following Examples can be appropriately changed within the range that does not depart from the gist of the present invention. Therefore, the range of the present invention should not be limitatively interpreted by the following Examples.

[Compound (Compound for Evaluation)]

<Synthesis of Compound (G-1)>

A compound (G-1) that is the specific compound was synthesized according to the following scheme.

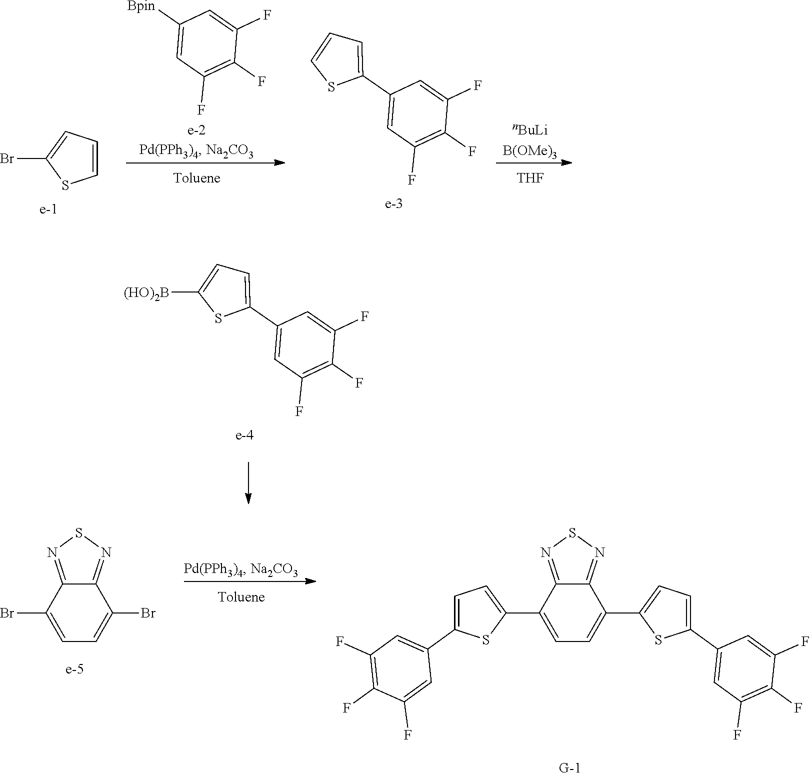

10 g of a compound (e-1) was charged into a three-necked flask, 1 equivalent of a compound (e-2), 2 equivalent of sodium carbonate, and 200 ml of toluene were added to the flask, and the mixed solution thus obtained was stirred. After stirring, the mixed solution was degassed under reduced pressure and substituted with nitrogen. Thereafter, 0.05 equivalent of tetrakis(triphenylphosphine)palladium(0) was added to the above-described mixed solution, and the was heated at 110° C. and stirred for 5 hours. Thereafter, the temperature of the mixed solution was lowered to room temperature (25° C.), and the mixed solution was separated into water and chloroform. The obtained organic phase was dried with magnesium sulfate and filtered. The obtained filtrate was concentrated and then purified by silica gel column chromatography to obtain 11.1 g of a compound (e-3).

11 g of the compound (e-3) was charged into a three-necked flask, 330 ml of tetrahydrofuran was further added to the flask under a nitrogen atmosphere, and the obtained mixed solution was stirred and cooled to −20° C. 1.03 equivalent of n-butyllithium in hexane was added to the above-described mixed solution, and the mixture was stirred for 30 minutes. 1.05 equivalent of trimethyl borate was added dropwise to the mixed solution and stirred for 30 minutes, and the mixed solution was then stirred at room temperature (25° C.) for 30 minutes. The mixed solution was separated into 0.5 N hydrochloric acid and ethyl acetate. The obtained organic phase was dried with magnesium sulfate and filtered. The obtained filtrate was concentrated and then purified by silica gel column chromatography to obtain 10.8 g of a compound (e-4).

4 g of a compound (e-5) was charged into a three-necked flask, 120 ml of toluene was further added to the flask under a nitrogen atmosphere, and the obtained mixed solution was stirred. 2.5 equivalent of the compound (e-4) and 3 equivalent of sodium carbonate were added to the mixed solution and stirred, and the mixed solution was then degassed under reduced pressure and substituted with nitrogen. Thereafter, 0.05 equivalent of tetrakis(triphenylphosphine)palladium(0) was added to the above-described mixed solution, and the mixture was heated at 110° C. and stirred for 8 hours. Thereafter, the temperature of the mixed solution was lowered to room temperature (25° C.), and was filtered. A residue (filtrate) thus obtained was suspended and washed with chloroform and then filtered. The residue (filtrate) thus obtained was dried under reduced pressure, and was sublimated and purified to obtain 3.05 g of a compound (G-1).

<Synthesis of Compound (I-2-5)>

A compound (I-2-5) that is a specific compound was synthesized according to the following scheme.

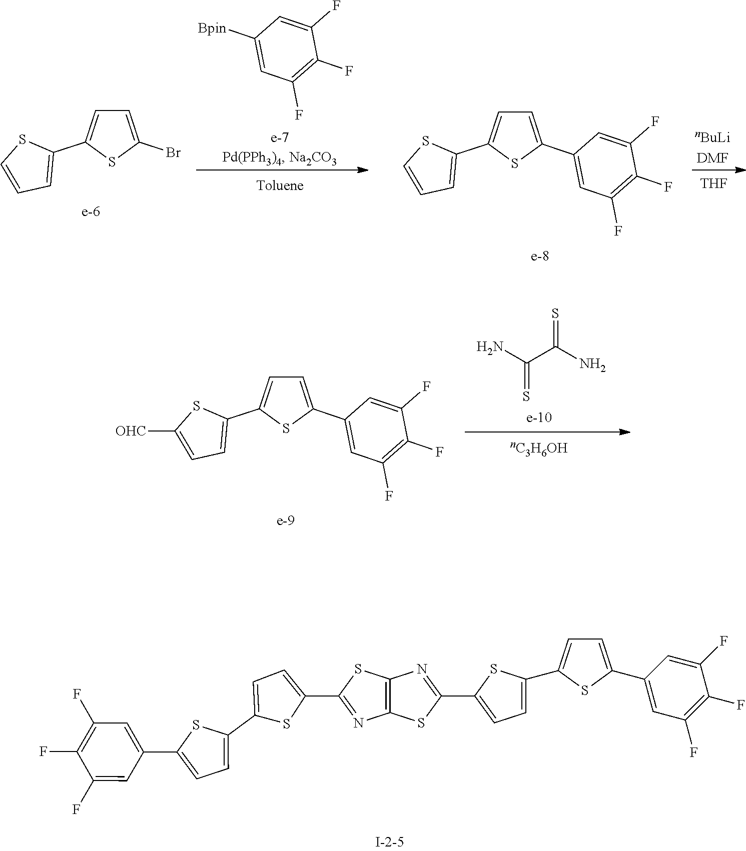

5 g of a compound (e-6) was charged into a three-necked flask, 1 equivalent of a compound (e-7), 2 equivalent of sodium carbonate, and 150 ml of toluene were further added to the flask, and the mixed solution thus obtained was stirred. After stirring, the mixed solution was degassed under reduced pressure and substituted with nitrogen. Thereafter, 0.05 equivalent of tetrakis(triphenylphosphine)palladium(0) was added to the above-described mixed solution, and the mixture was heated at 110° C. and stirred for 8 hours. Thereafter, the temperature of the mixed solution was lowered to room temperature (25° C.), and the mixed solution was separated into water and chloroform. The obtained organic phase was dried with magnesium sulfate and filtered. The obtained filtrate was concentrated and then purified by silica gel column chromatography to obtain 5.13 g of a compound (e-8).

5 g of the compound (e-8) was charged into a three-necked flask, 200 ml of tetrahydrofuran was further added to the flask under a nitrogen atmosphere, and the obtained mixed solution was stirred and cooled to −20° C. 1.03 compound equivalent of n-butyllithium in hexane was added to the above-described mixed solution, and the mixture was stirred at 0° C. for 30 minutes. Thereafter, 1.05 equivalent of dimethylformamide was added dropwise to the above-described mixed solution, and the mixture was stirred for 30 minutes and then stirred at room temperature (25° C.) for 1 hour. A saturated ammonium chloride aqueous solution and chloroform were added to the above-described mixed solution, the mixture was separated and extracted, and an organic phase thus obtained was dried with magnesium sulfate and then filtered. The obtained filtrate was concentrated and then purified by silica gel column chromatography to obtain 4.82 g of a compound (e-9).

4.8 g of the compound (e-9) was charged into a three-necked flask, and 0.5 equivalent of a compound (e-10) and 192 ml of n-propanol were further added to the flask under a nitrogen atmosphere to obtain a mixed solution, and the obtained mixed solution was heated and stirred at 100° C. for 8 hours. Thereafter, the temperature of the mixed solution was lowered to room temperature (25° C.), and was filtered. A residue (filtrate) thus obtained was suspended and washed with chloroform and then filtered. The residue (filtrate) thus obtained was dried under reduced pressure, and was sublimated and purified to obtain 4.62 g of a compound (I-2-5).

Other specific compounds were also synthesized with reference to the above-described synthesis method.

The measurement result of each synthesized specific compound by electrospray ionization mass spectrometry (ESI-MS) was as follows.

| | ESI-MS |
|---|---|
| | measurement |
| | result |
| Compound | (m/z (M⁺)) |
| F-1 | 511.96 |
| F-2 | 452.05 |
| F-3 | 488.03 |
| F-4 | 400.02 |
| F-5 | 588.02 |
| F-6 | 491.79 |
| F-7 | 455.81 |
| G-1 | 599.99 |
| G-2 | 524.01 |
| G-3 | 559.99 |
| G-4 | 335.97 |
| G-5 | 454.04 |
| G-6 | 502.04 |
| G-7 | 456.03 |
| H-1 | 417.89 |
| H-2 | 469.92 |
| H-3 | 458.00 |
| H-4 | 593.98 |
| H-5 | 461.76 |
| H-6 | 405.97 |
| I-1-1 | 355.93 |
| I-1-2 | 493.99 |
| I-1-3 | 565.95 |
| I-1-4 | 637.91 |
| I-1-5 | 461.98 |
| I-2-1 | 505.90 |
| I-2-2 | 519.91 |
| I-2-3 | 657.96 |
| I-2-4 | 693.94 |
| I-2-5 | 729.92 |
| I-2-6 | 801.89 |
| I-2-7 | 671.97 |
| I-2-8 | 625.96 |
| I-3-1 | 569.95 |
| I-4-1 | 553.9 |
| I-4-2 | 741.94 |
| I-5-1 | 453.87 |
| I-5-2 | 605.93 |
| I-5-3 | 641.91 |
| I-5-4 | 677.89 |
| I-5-5 | 705.92 |
| I-5-6 | 619.94 |
| I-5-7 | 573.93 |

The measurement results of compounds J-1 to J-23 and compounds K-1 to K-19 among the synthesized specific compounds by LDI-MS (soft laser desorption ionization mass analysis) were as follows.

| | LDI-MS |
|---|---|
| | measurement |
| | result |
| Compound | (m/z (M⁺)) |
| J-1 | 653.95 |
| J-2 | 591.96 |
| J-3 | 662.08 |
| J-4 | 596.99 |
| J-5 | 618.01 |
| J-6 | 653.98 |
| J-7 | 632.05 |
| J-8 | 626.00 |
| J-9 | 773.92 |
| J-10 | 643.95 |
| J-11 | 644.03 |
| J-12 | 677.99 |
| J-13 | 680.04 |
| J-14 | 679.89 |
| J-15 | 713.93 |
| J-16 | 677.99 |
| J-17 | 714.07 |
| J-18 | 714.10 |
| J-19 | 677.80 |
| J-20 | 620.01 |
| J-21 | 690.09 |
| J-22 | 655.91 |
| J-23 | 725.90 |
| K-1 | 653.95 |
| K-2 | 653.95 |
| K-3 | 653.95 |
| K-4 | 675.76 |
| K-5 | 640.03 |
| K-6 | 640.03 |
| K-7 | 640.03 |
| K-8 | 703.97 |
| K-9 | 667.96 |
| K-10 | 667.96 |
| K-11 | 667.96 |
| K-12 | 691.79 |
| K-13 | 579.85 |
| K-14 | 667.96 |
| K-15 | 656.05 |
| K-16 | 656.05 |
| K-17 | 455.99 |
| K-18 | 672.00 |
| K-19 | 659.96 |

The specific compound and comparative compound used in a test are shown below.

Hereinafter, the specific compound and the Comparative compound are collectively referred to as a compound for evaluation.

<Specific Compound Used in Test>

Specific compounds used in a test are as follows.

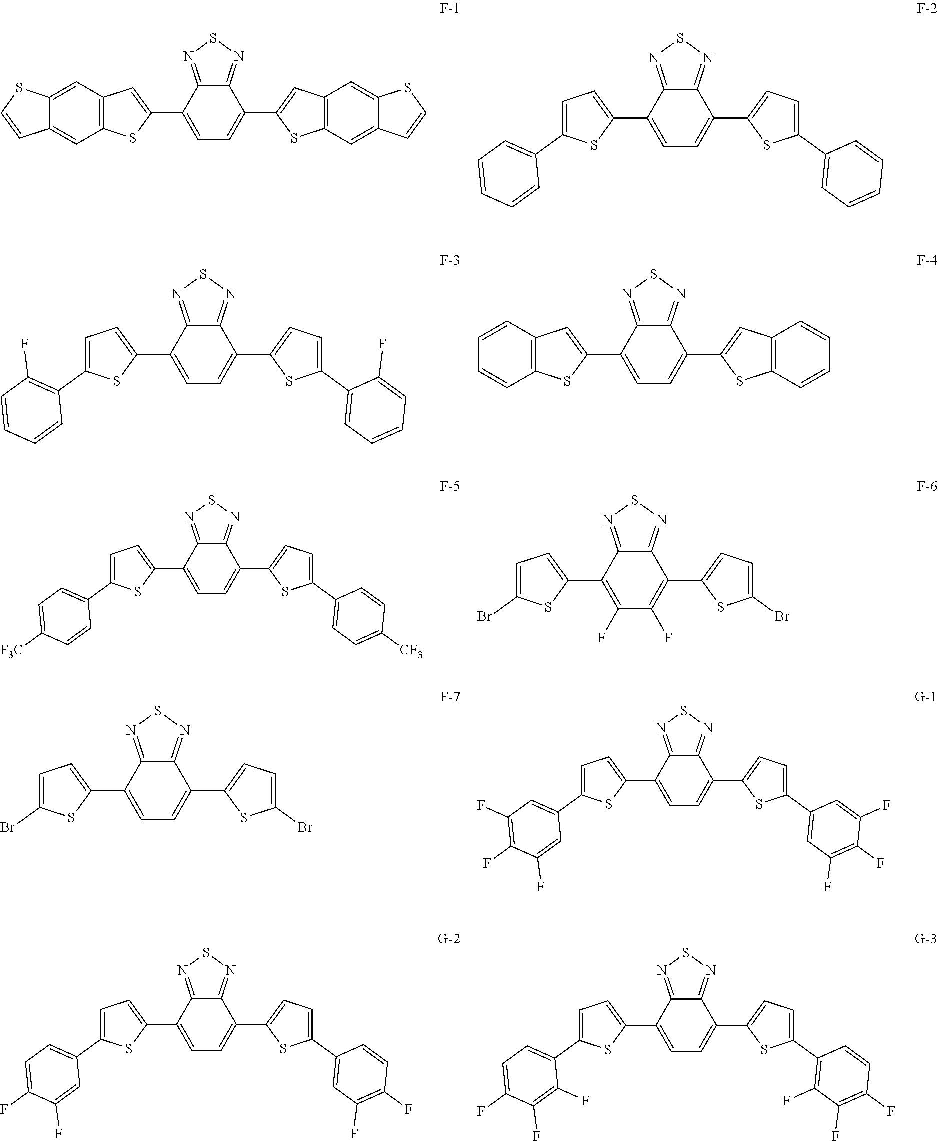

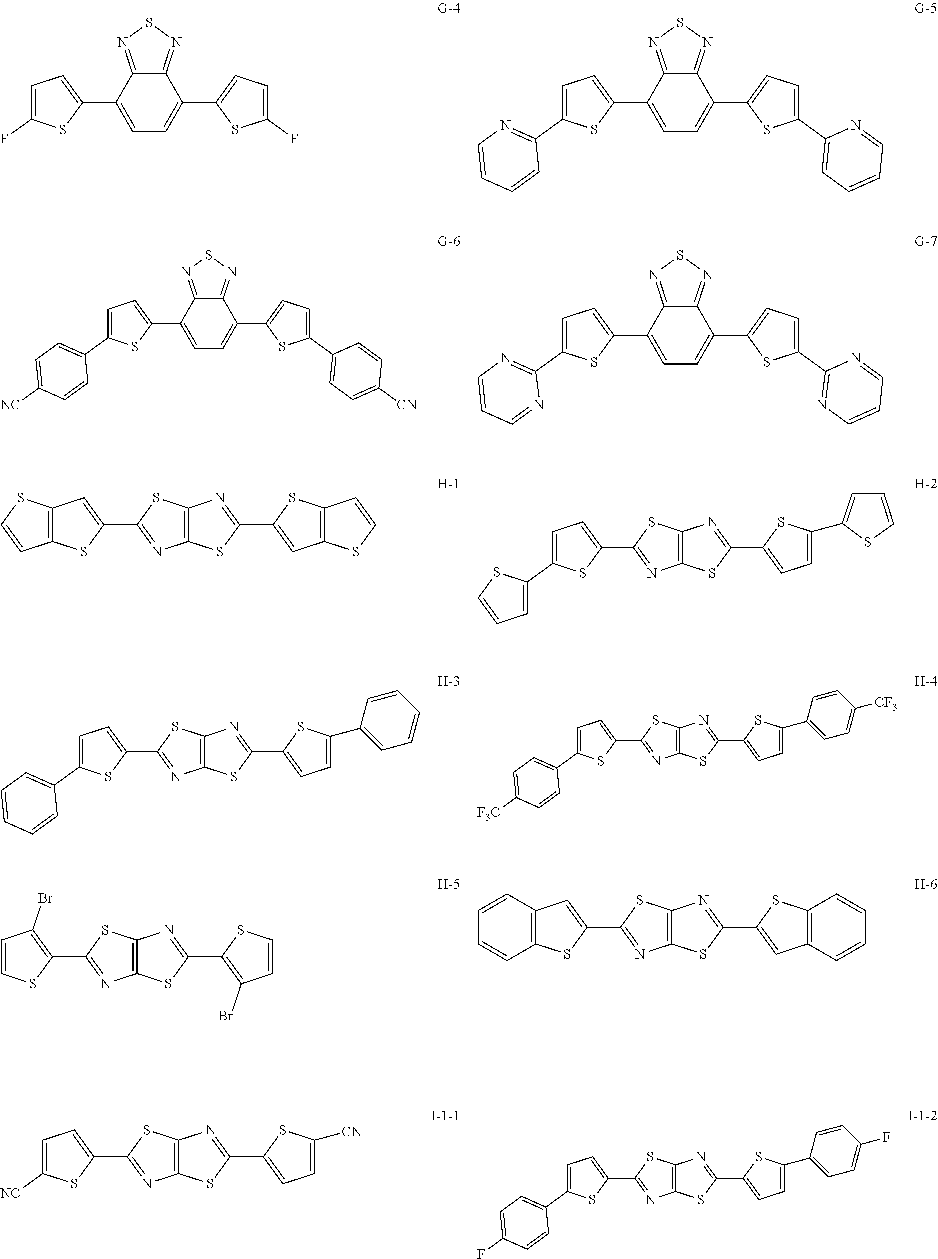

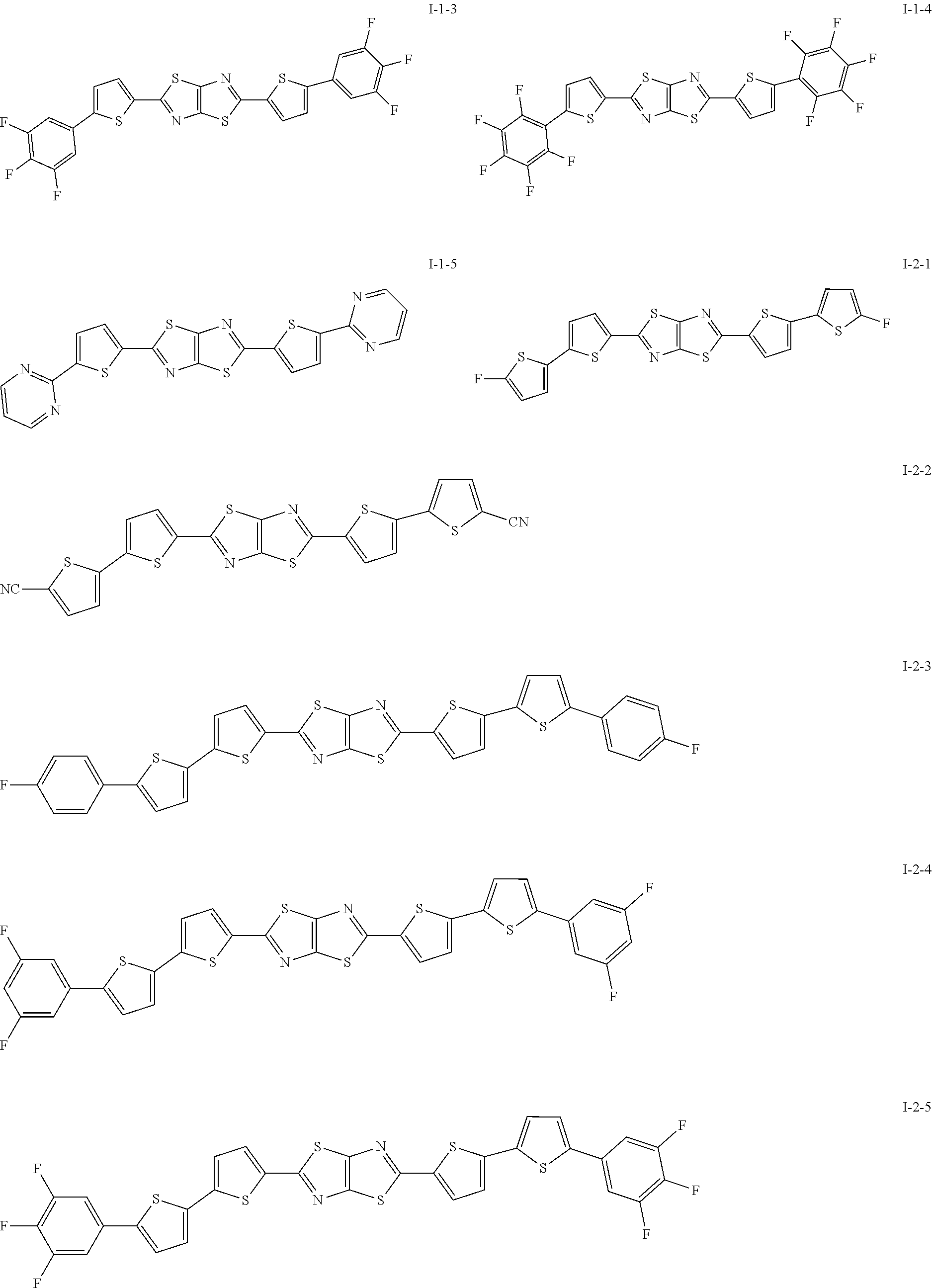

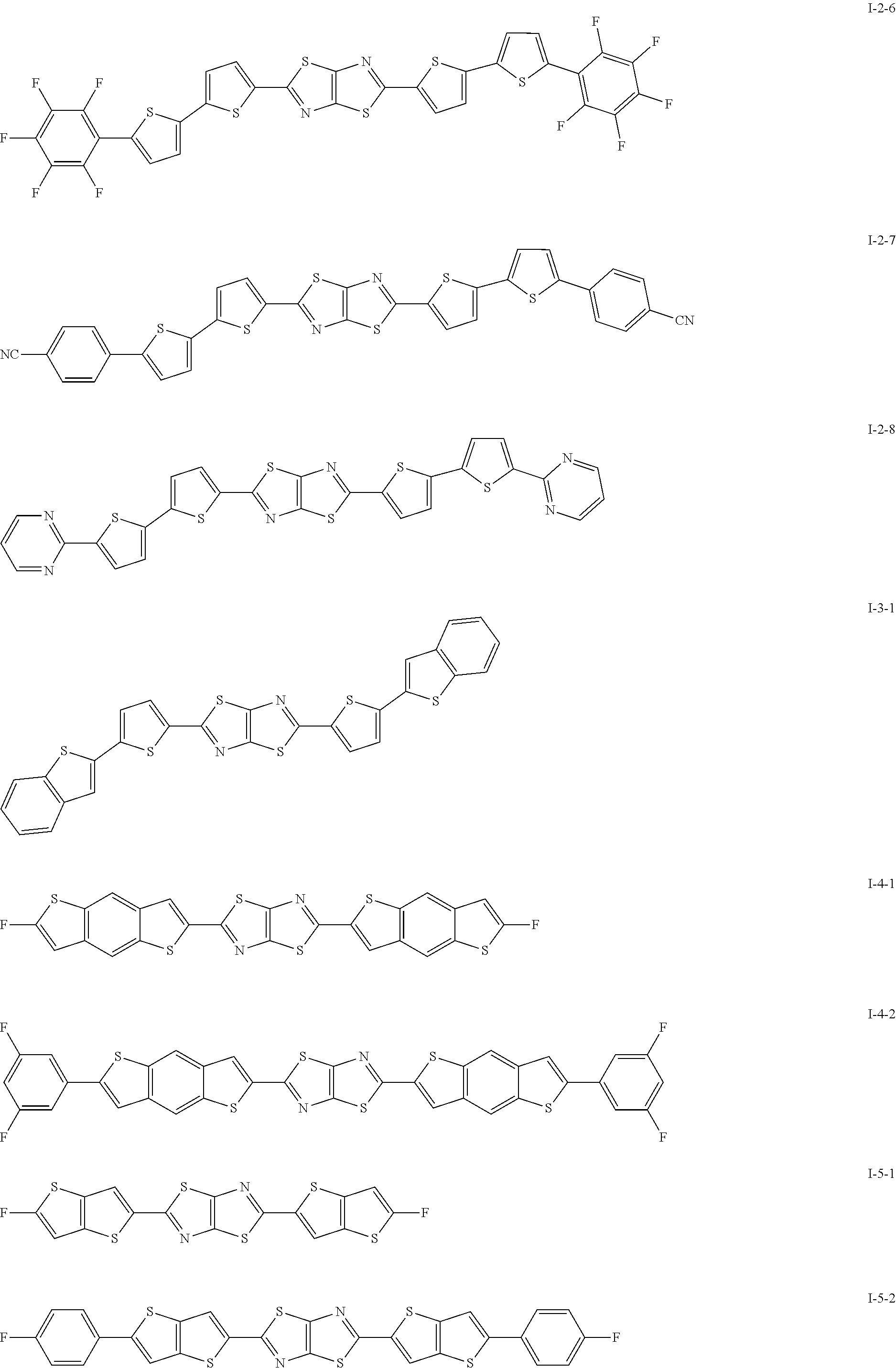

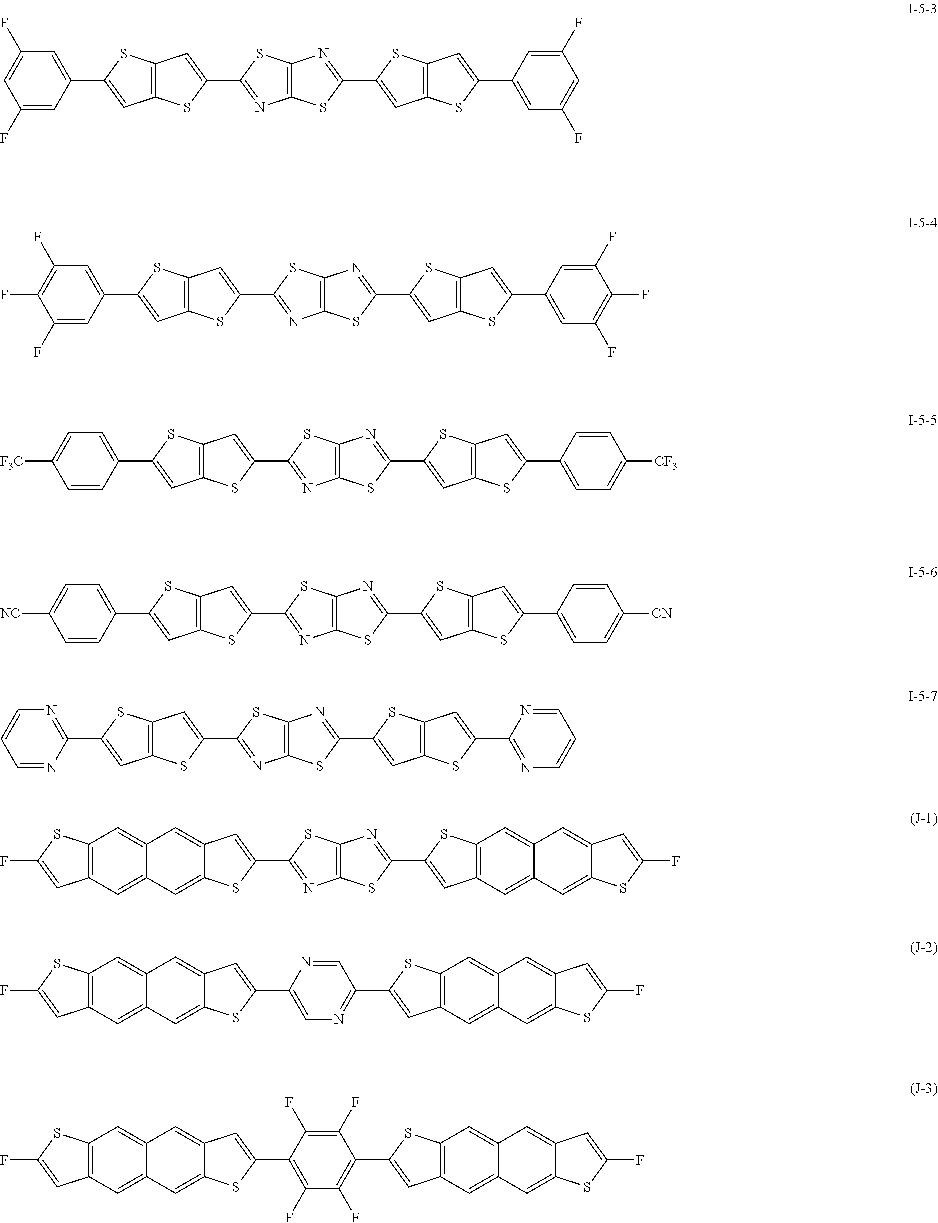

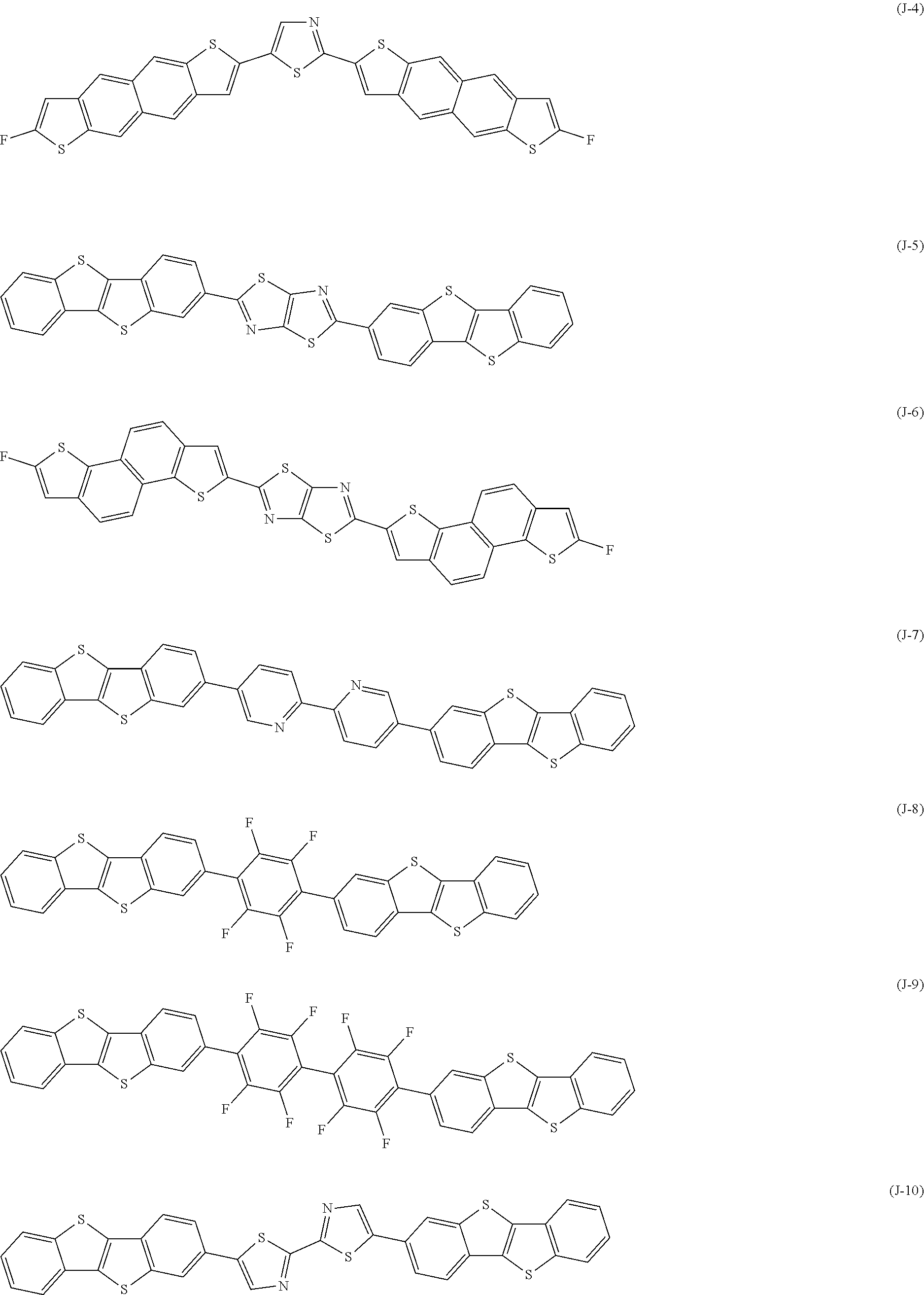

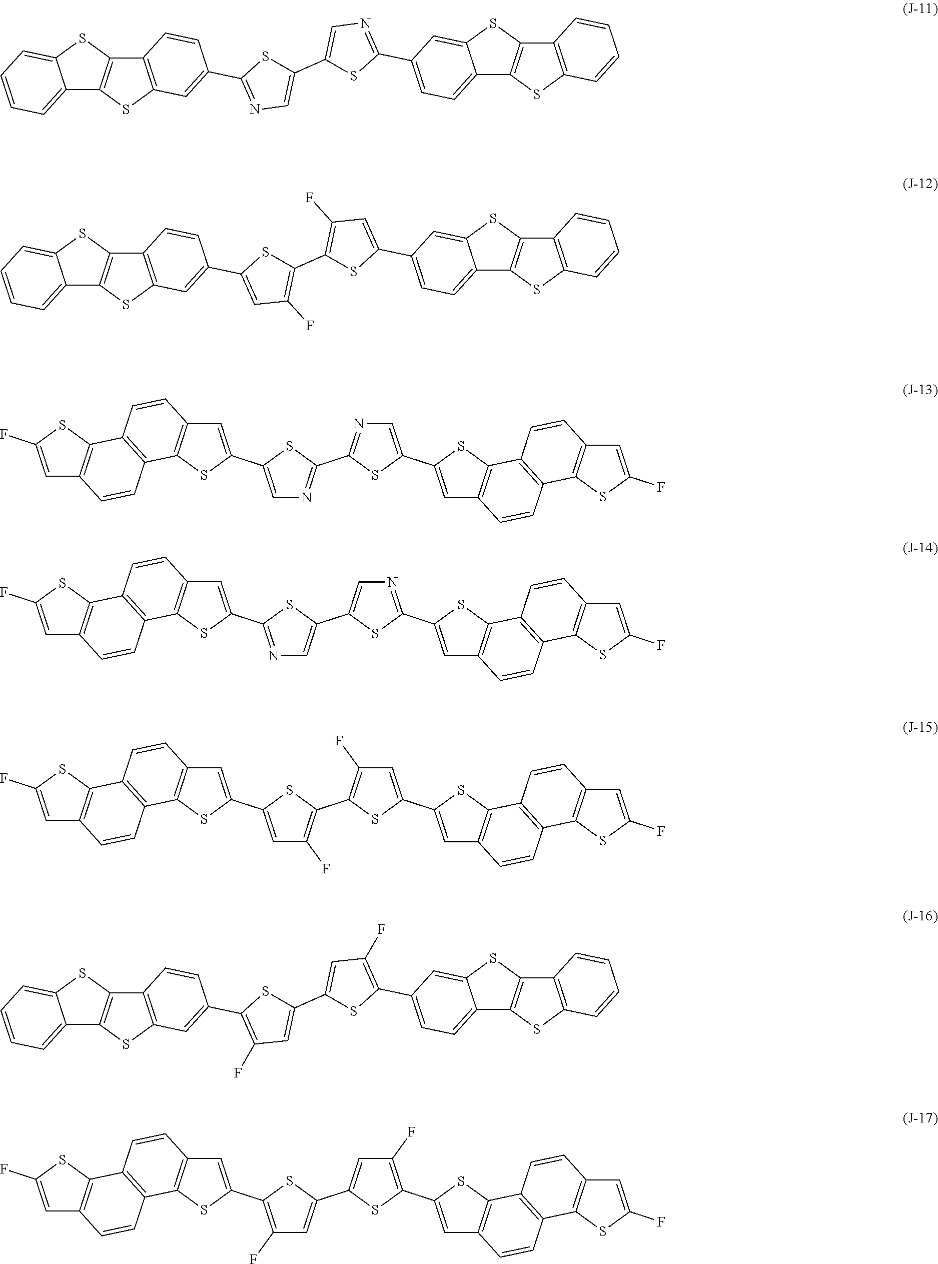

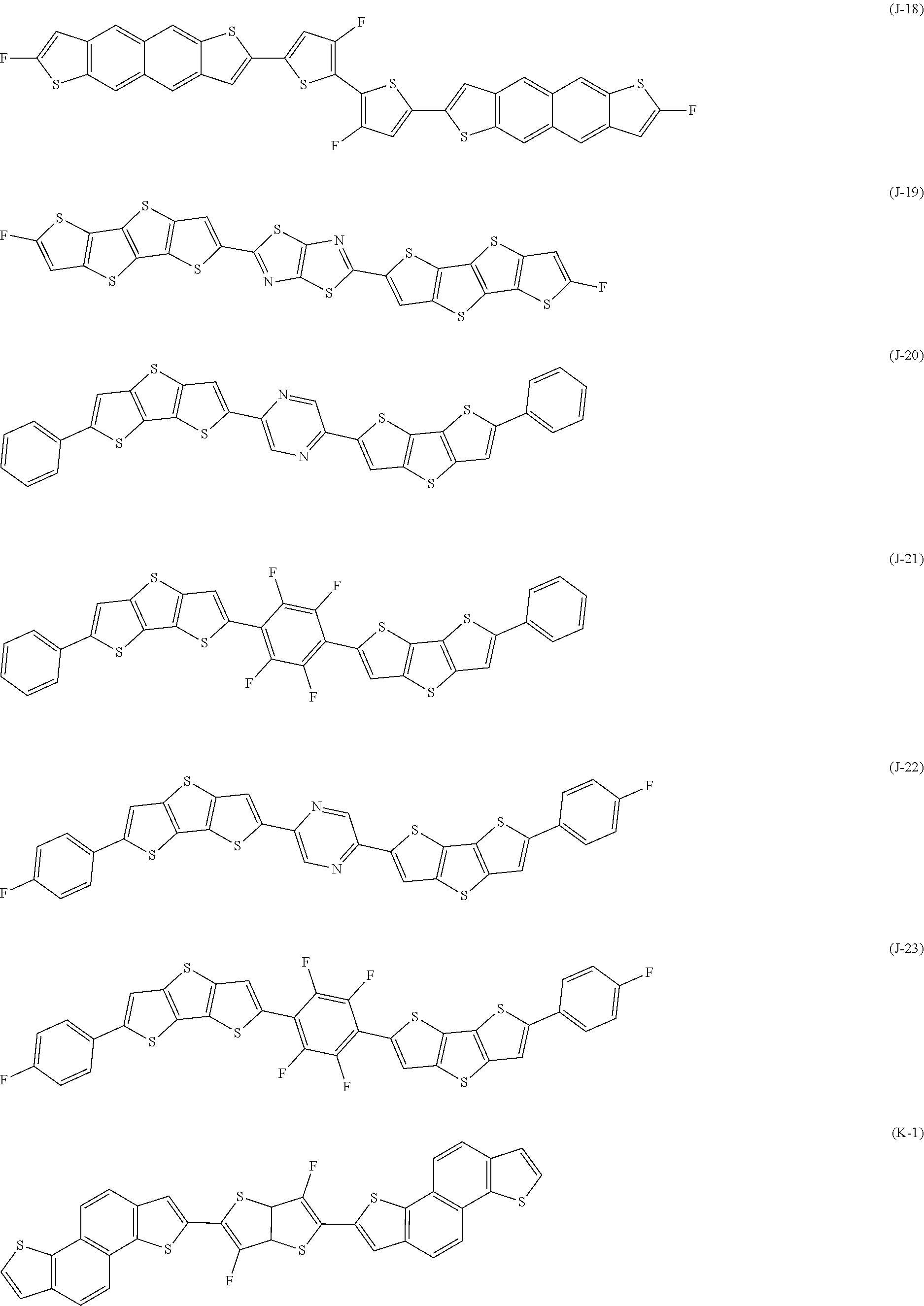

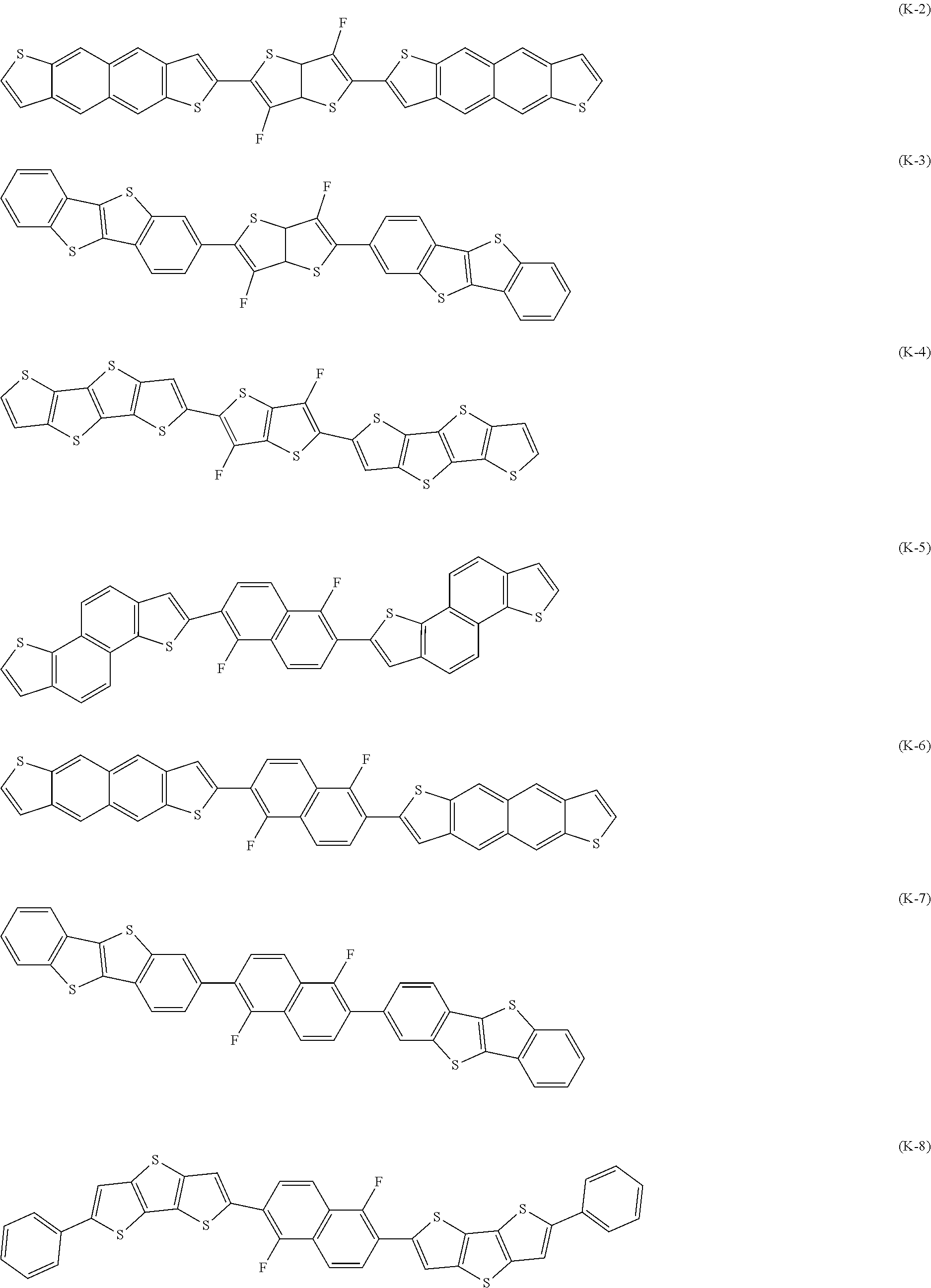

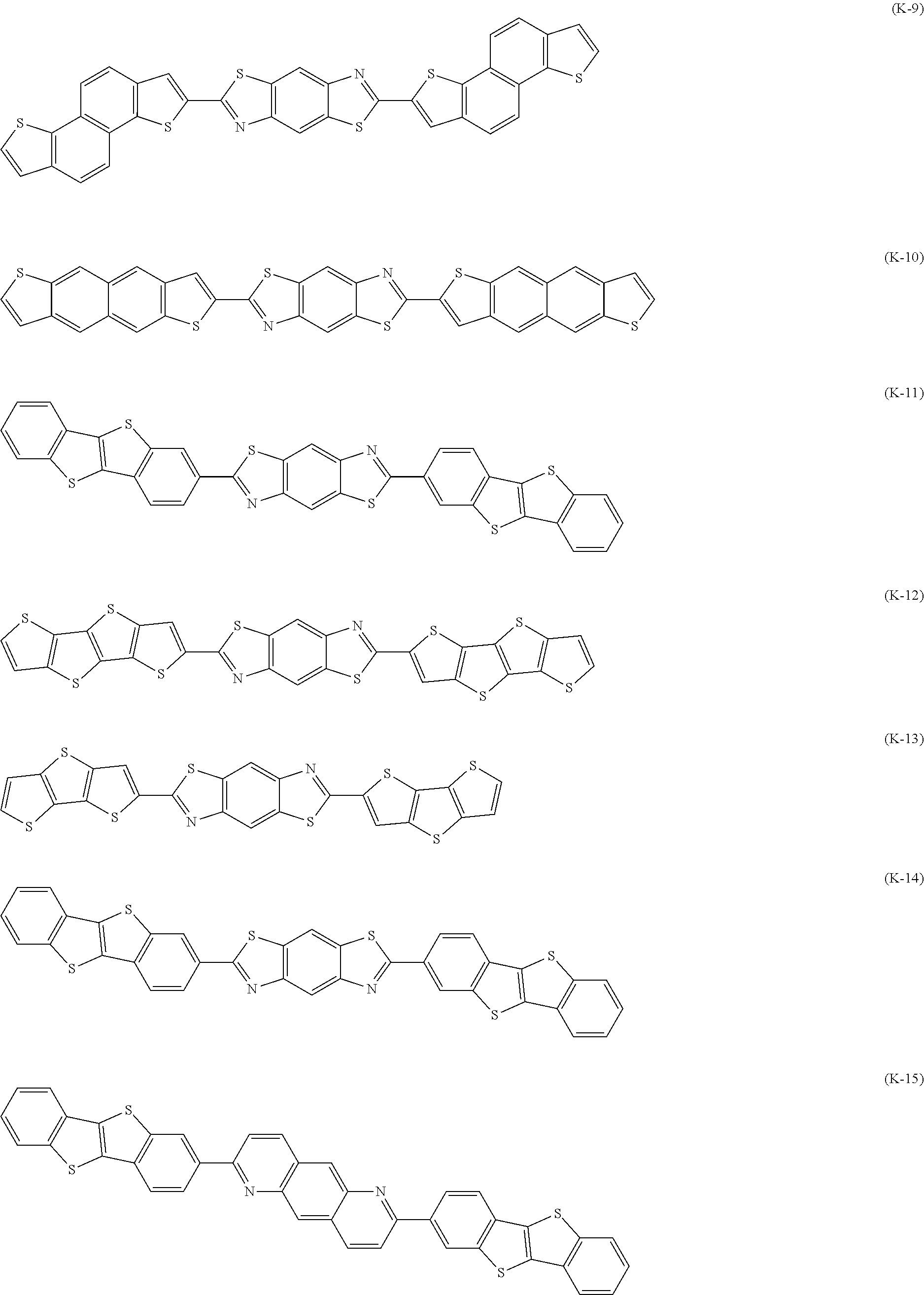

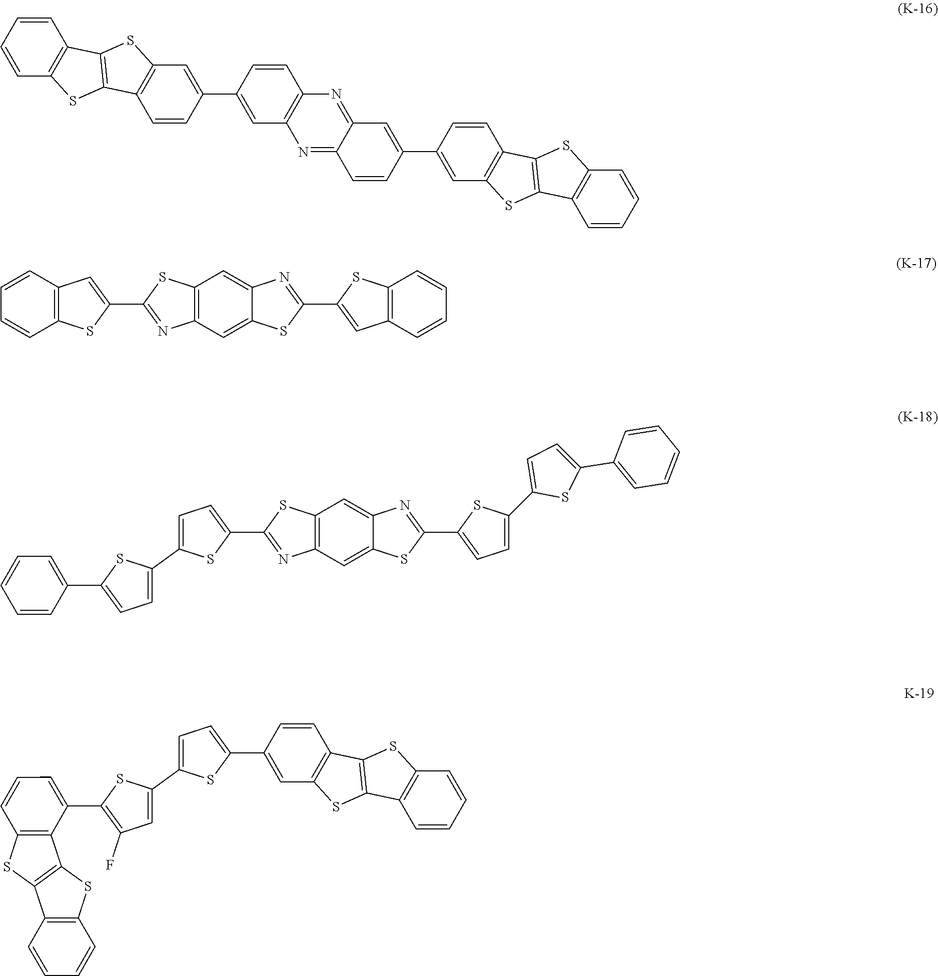

<Comparative Compound Used in Test>

Comparative compounds used in a test are as follows.

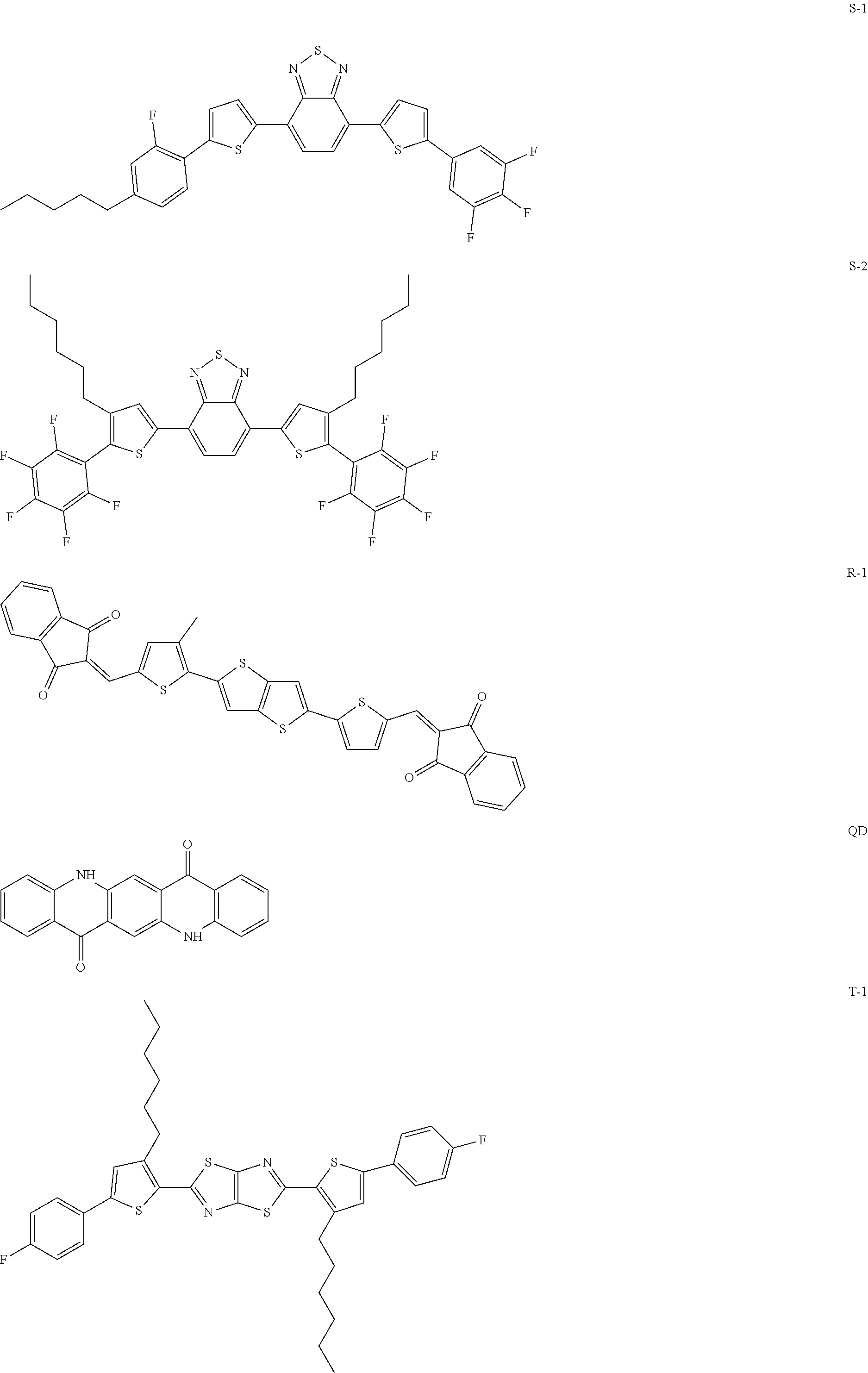 [SMILES: CCCCCc1ccc(-c2ccc(-c3ccc(-c4ccc(-c5cc(F)c(F)c(F)c5)s4)c4nsnc34)s2)c(F)c1 ; CCCCCCc1cc(-c2ccc(-c3cc(CCCCCC)c(-c4c(F)c(F)c(F)c(F)c4F)s3)c3nsnc23)sc1-c1c(F)c(F)c(F)c(F)c1F ; Cc1cc(C=C2C(=O)c3ccccc3C2=O)sc1-c1cc2sc(-c3ccc(C=C4C(=O)c5ccccc5C4=O)s3)cc2s1 ; O=c1c2ccccc2[nH]c2cc3c(=O)c4ccccc4[nH]c3cc12 ; CCCCCCc1cc(-c2ccc(F)cc2)sc1-c1nc2sc(-c3sc(-c4ccc(F)cc4)cc3CCCCCC)nc2s1]

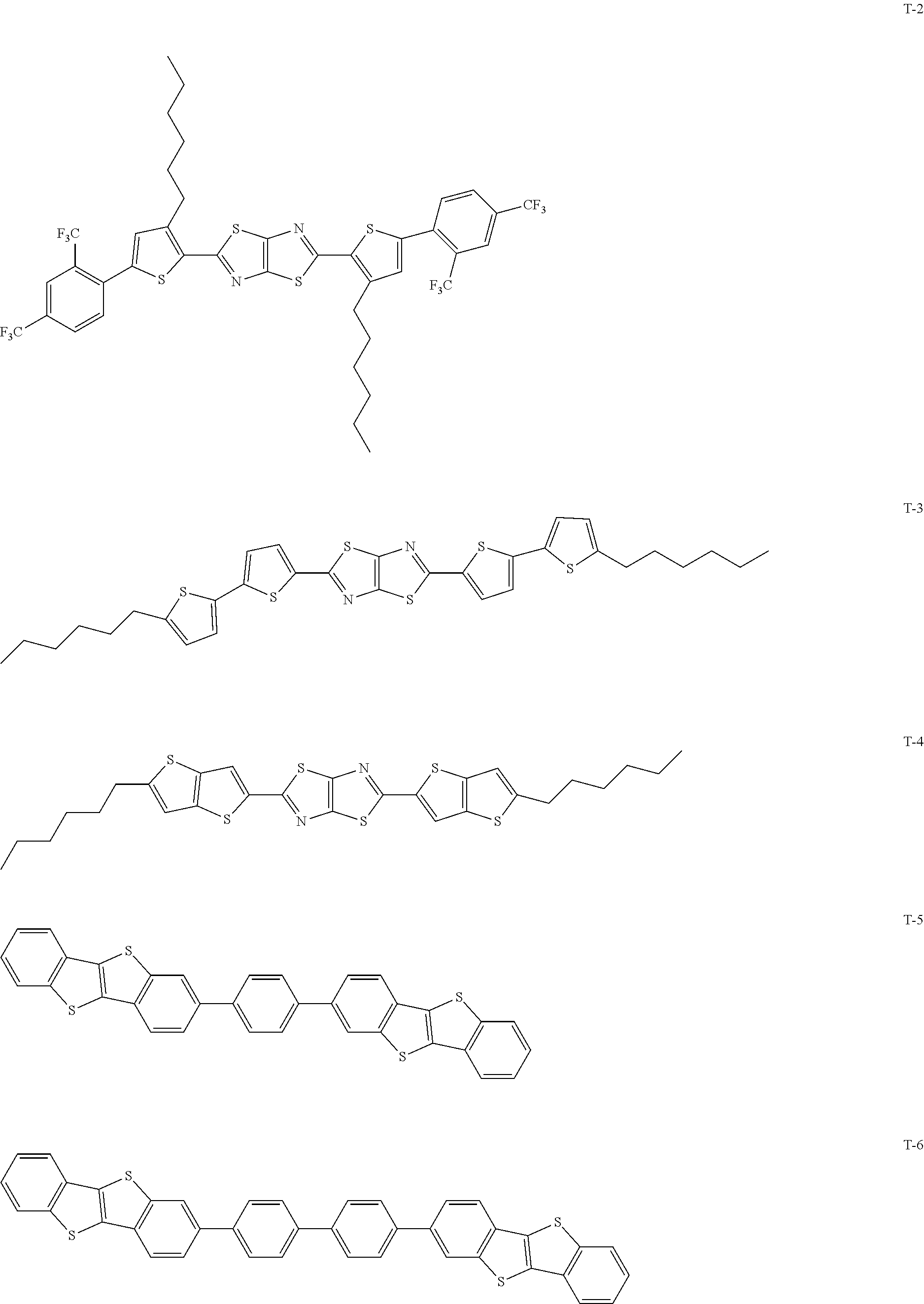

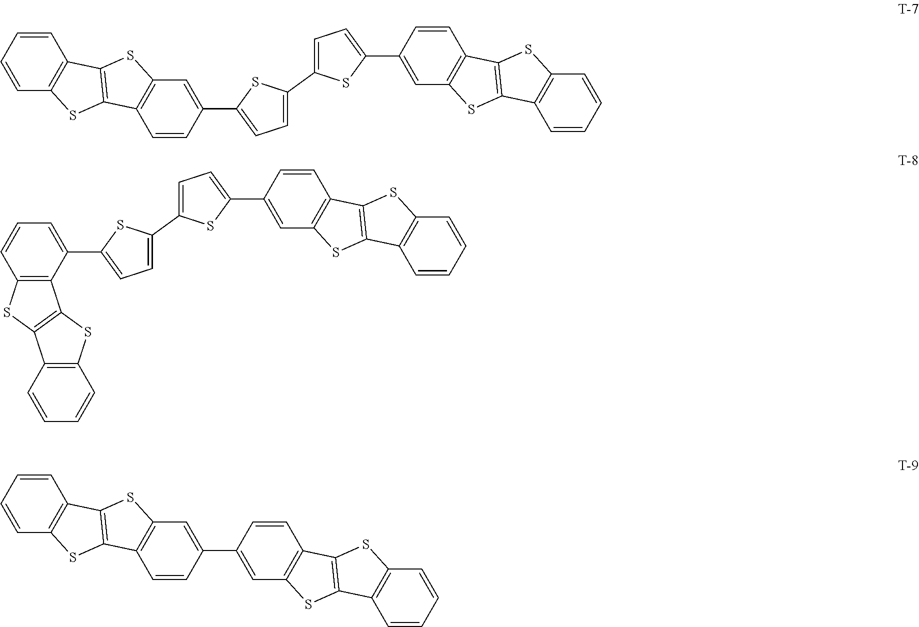

[Coloring Agent (Coloring Agent for Evaluation)]

Coloring agents shown below were coloring agents for evaluation used in the evaluation in Examples, and were used in the production of photoelectric conversion elements described later.

A compound (B-1), which is a coloring agent for evaluation, is the same compound as a compound (QD), which is a comparative compound.

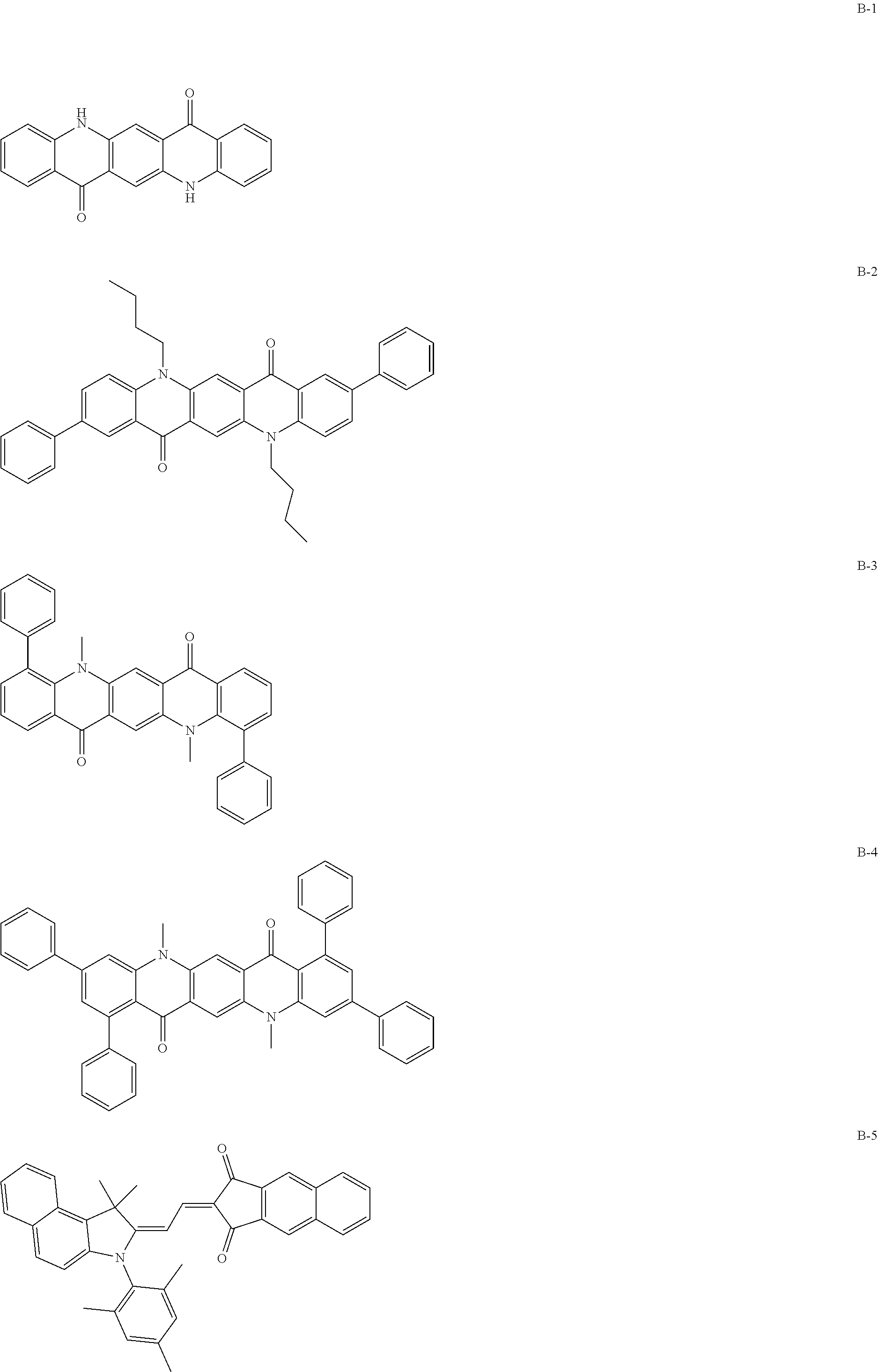 [SMILES: O=c1c2ccccc2[nH]c2cc3c(=O)c4ccccc4[nH]c3cc12 ; CCCCn1c2ccc(-c3ccccc3)cc2c(=O)c2cc3c(cc21)c(=O)c1cc(-c2ccccc2)ccc1n3CCCC ; Cn1c2cc3c(=O)c4cccc(-c5ccccc5)c4n(C)c3cc2c(=O)c2cccc(-c3ccccc3)c21 ; Cn1c2cc3c(=O)c4c(-c5ccccc5)cc(-c5ccccc5)cc4n(C)c3cc2c(=O)c2c(-c3ccccc3)cc(-c3ccccc3)cc21 ; Cc1cc(C)c(N2/C(=C/C=C3C(=O)c4cc5ccccc5cc4C3=O)C(C)(C)c3c2ccc2ccccc32)c(C)c1]

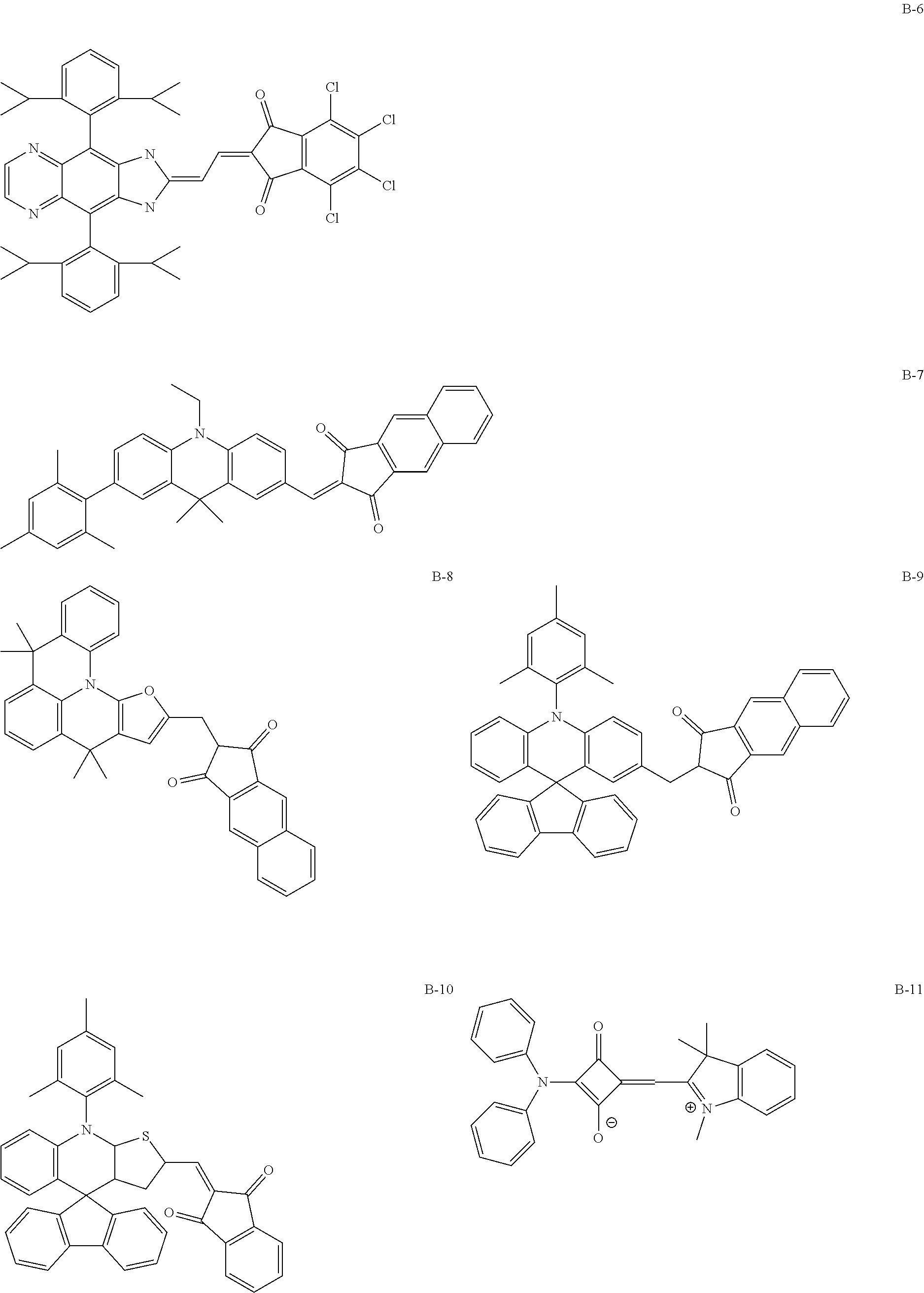

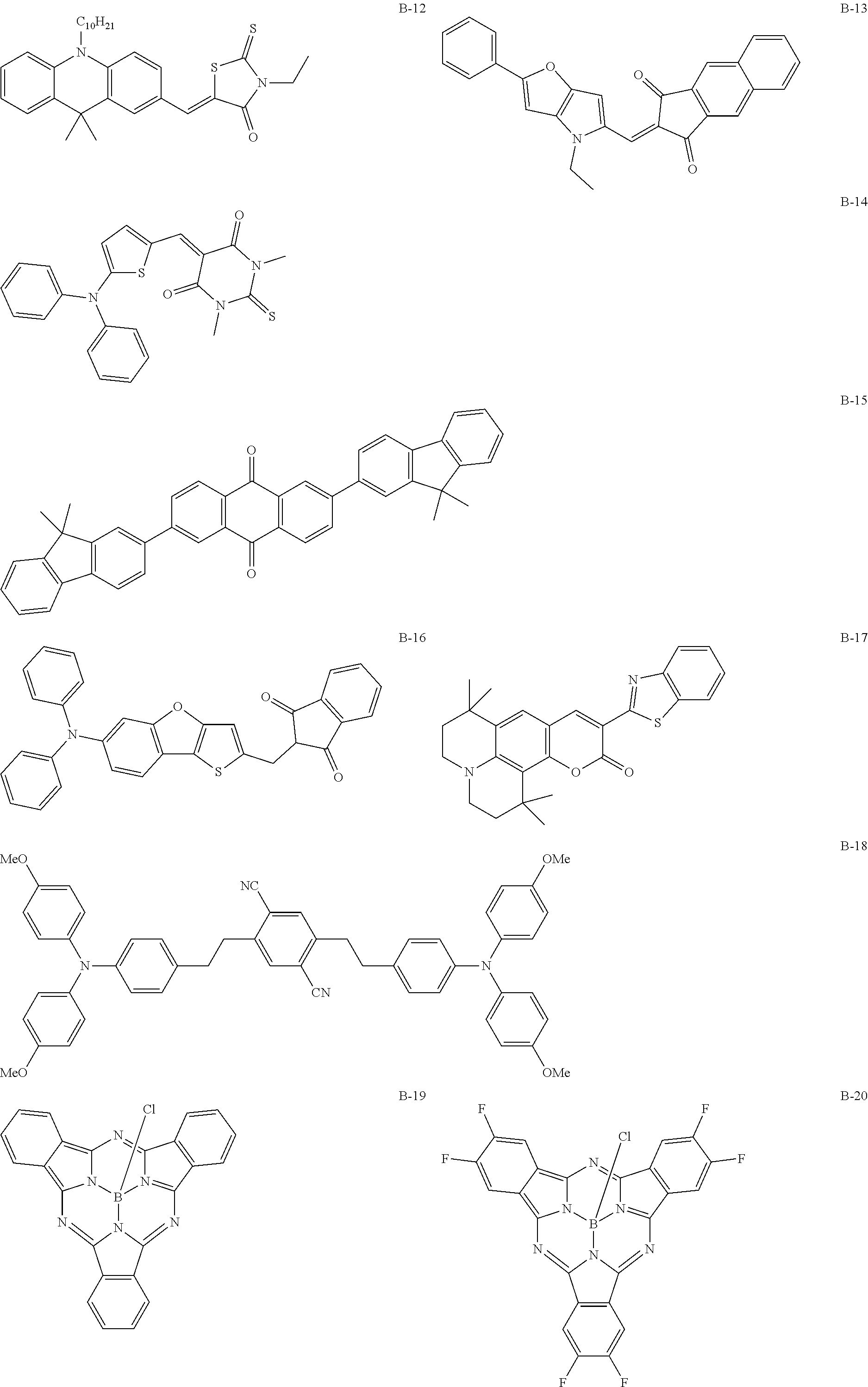

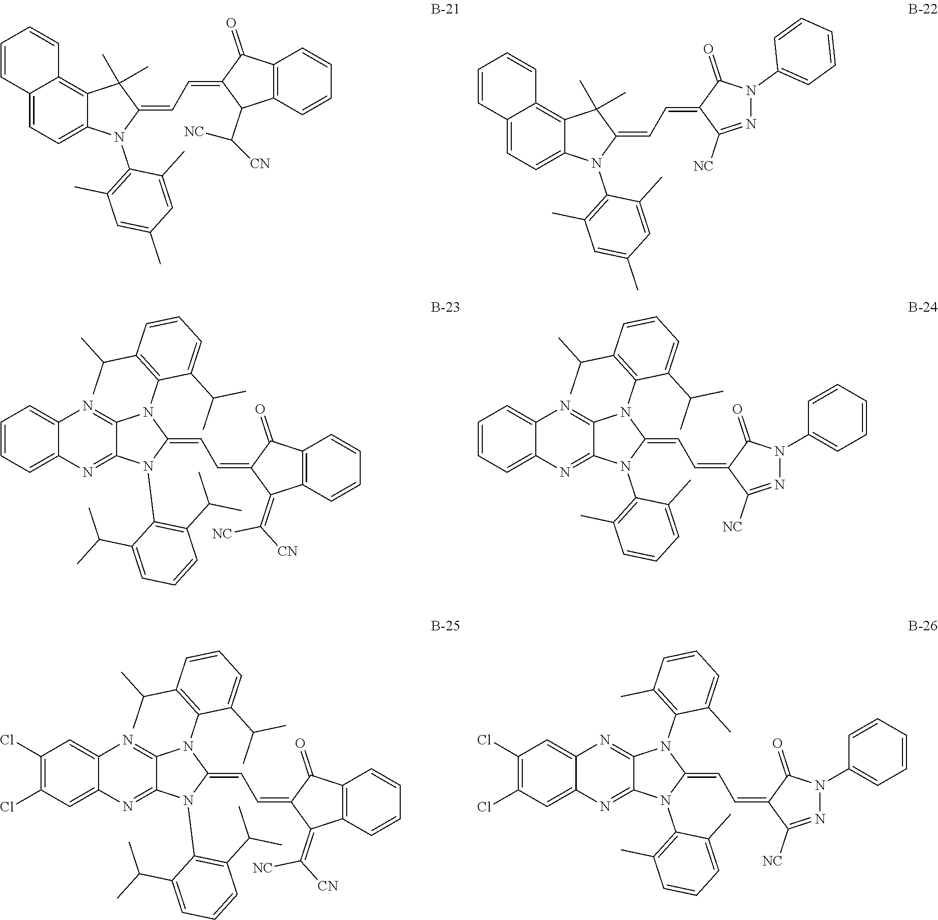

[n-Type Semiconductor Material]

Fullerene C₆₀ was used for the production of photoelectric conversion elements described later, as a n-type semiconductor material used for evaluations.

[Test]

### Examples and Comparative Examples: Production of Photoelectric Conversion Element

The photoelectric conversion element of the form illustrated in FIG. 2 was produced using the obtained compounds. Here, the photoelectric conversion element includes a lower electrode 11, an electron blocking film 16A, a photoelectric conversion film 12, a positive hole blocking film 16B, and an upper electrode 15.

Specifically, an amorphous ITO was formed into a film on a glass substrate by a sputtering method to form the lower electrode 11 (thickness: 30 nm). Furthermore, a compound (C-1) described below was formed into a film on the lower electrode 11 by a vacuum thermal vapor deposition method to form the electron blocking film 16A (thickness: 10 nm).

Furthermore, in a state where the temperature of the substrate is controlled to 25° C., the compound for evaluation (any of the above-described compounds for evaluation), the n-type semiconductor material (fullerene C₆₀), and the coloring agent (any of the coloring agents for evaluation described above) were set on the electron blocking film 16A at a vapor deposition rate of 2.0 Å/sec, and co-vapor deposition was carried out by a vacuum vapor deposition method so that each of them had a thickness of 100 nm in terms of a single layer, and a photoelectric conversion film 12 of which the total thickness was 300 nm, which is the mixture layer, was formed (photoelectric conversion film forming step). However, in Comparative Example 11, the photoelectric conversion film 12, which is the mixture layer having a total film thickness of 200 nm, was formed without using the n-type semiconductor material.

Furthermore, a compound (C-2) described below was formed into a film on the photoelectric conversion film 12 to form the positive hole blocking film 16B (thickness: 10 nm).

Furthermore, amorphous ITO was formed into a film on the positive hole blocking film 16B by a sputtering method to form the upper electrode 15 (the transparent conductive film) (thickness: 10 nm). A SiO film was formed as a sealing layer on the upper electrode 15 by a vacuum vapor deposition method, and thereafter, an aluminum oxide (Al₂O₃) layer was formed thereon by an atomic layer chemical vapor deposition (ALCVD) method to produce a photoelectric conversion element (simply referred to as an “element”) obtained in each of Examples or Comparative Examples.

By using the same combination of the compounds for evaluation and the coloring agents for evaluation, 10 elements of the same kind prepared in the same procedure were produced and subjected to evaluations described below.

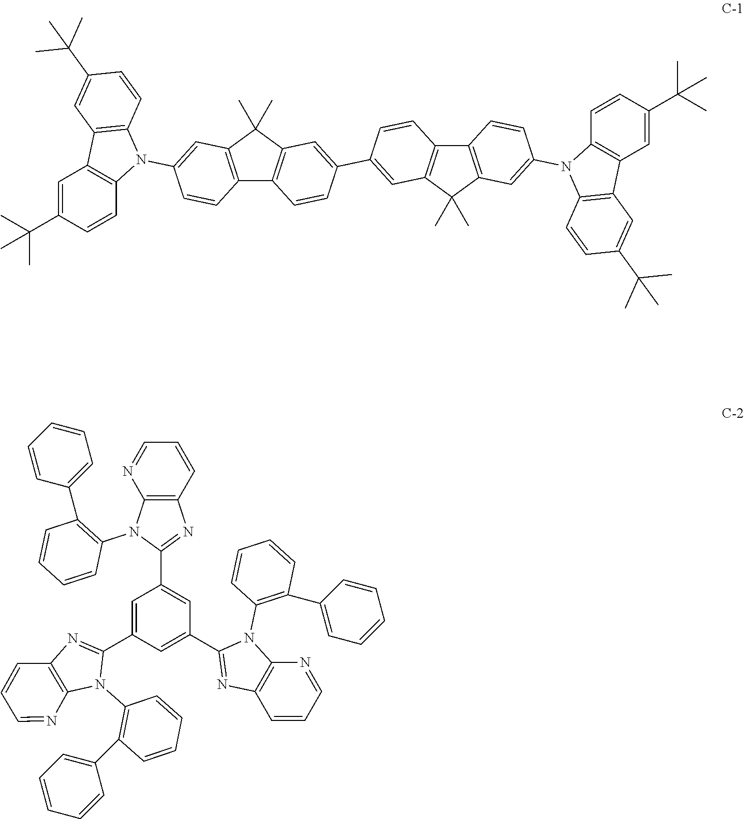 [SMILES: CC(C)(C)c1ccc2c(c1)c1cc(C(C)(C)C)ccc1n2-c1ccc2c(c1)C(C)(C)c1cc(-c3ccc4c(c3)C(C)(C)c3cc(-n5c6ccc(C(C)(C)C)cc6c6cc(C(C)(C)C)ccc65)ccc3-4)ccc1-2 ; c1ccc(-c2ccccc2-n2c(-c3cc(-c4nc5cccnc5n4-c4ccccc4-c4ccccc4)cc(-c4nc5cccnc5n4-c4ccccc4-c4ccccc4)c3)nc3cccnc32)cc1]

<Evaluation of Photoelectric Conversion Efficiency (Sensitivity)>

The drive of each of the obtained elements was confirmed. A voltage was applied to each element to have an electric field strength of 2.0×10⁵ V/cm. Thereafter, light was emitted from the upper electrode (transparent conductive film) side to evaluate the photoelectric conversion efficiency (external quantum efficiency) at 450 nm. The external quantum efficiency was measured using a constant energy quantum efficiency measuring device manufactured by Optel Co., Ltd. The amount of light emitted was 50 μW/cm².

An external quantum efficiency of each of the ten manufactured elements of the same kind was measured, and an average external quantum efficiency was used as the external quantum efficiency of the elements of that kind.

The external quantum efficiency was evaluated as “A” in a case of 92% or more, evaluated as “B” in a case of 90% or more and less than 92%, evaluated as “C” in a case of 87% or more and less than 90%, “D” in a case of 85% or more and less than 87%, evaluated as “E” in a case of 82% or more and less than 85%, and evaluated as “F” in a case of less than 82%.

Practically, evaluations of “A”, “B”, “C”, and “D” are preferable, “A”, “B”, and “C” are more preferable, and “A” and “B” are still more preferable.

<Measurement of Responsiveness>

Each of the obtained elements was used to perform the following evaluation of responsiveness.

Specifically, a voltage was applied to each element to have a strength of 2.0×10⁵ V/cm. Thereafter, light emitting diodes (LEDs) were instantaneously turned on to emit light from an upper electrode (transparent conductive film) side, and a photocurrent at that time was measured with an oscilloscope. At this time, assuming that the current intensity (signal intensity) before light emission was 0%, and the maximum signal intensity measured by light emission was 100%, a time (rise time) until the signal intensity reached from 0% to 97% was determined for each element.

The rise time of each of the produced 10 elements of the same kind was measured, and an average rise time was used as the rise time of the elements of that kind.

A relative value of a rise time of each element was obtained, where a rise time of an element of Comparative Example 1 was set to 1.

The responsiveness was evaluated as “AA” in a case where the relative value of the rise time is less than 0.10, evaluated as “A” in a case of 0.10 or more and less than 0.15, evaluated as “B” in a case of 0.15 or more and less than 0.20, evaluated as “C” in a case of 0.20 or more and less than 0.25, evaluated as “D” in a case of 0.25 or more and less than 0.30, evaluated as “E” in a case of 0.30 or more and less than 1.00, and evaluated as “F” in a case of 1.00 or more.

Practically, “A”, “B”, “C”, and “D” are preferable, “A”, “B”, and “C” are more preferable, and “A” and “B” are still more preferable.

<Evaluation of Preventing Properties of Variation in Response>

A rise time was measured for each of the produced ten elements of the same kind in the same manner as in the above-described <Measurement of Responsiveness>. An average value of the rise times of the 10 elements was standardized as 1, and the standard deviation of the rise times of the 10 elements was obtained to evaluate preventing properties of variation in response.

The preventing properties of variation in response were evaluated as “A” in a case where the standard deviation is less than 0.02, evaluated as “B” in a case where the standard deviation is 0.02 or more and less than 0.03, evaluated as “C” in a case where the standard deviation is 0.03 or more and less than 0.04, evaluated as “D” in a case where the standard deviation is 0.04 or more and less than 0.05, evaluated as “E” in a case where the standard deviation is 0.05 or more and less than 0.10, and evaluated as “F” in a case where the standard deviation is 0.10 or more.

The standard deviation can be calculated by the following Equation.

s: Standard deviation

n: 10

x: 1

xᵢ: Rise time of i-th element in a case where the average value of the rise times of the 10 elements is standardized as 1.

$$s = \sqrt{\frac{1}{n} {\sum }_{i = 1}^{n} {( {x}_{i} - x )}^{2}}$$

<Evaluation of Heat Resistance>

Each of the obtained elements was heat-treated in a glove box at 160° C. for 1 hour. Thereafter, the external quantum efficiency was evaluated by the same method as in the above-described <Evaluation of Photoelectric Conversion Efficiency (Sensitivity)>. Assuming that the external quantum efficiency before heat-treated was 1, a relative value of the external quantum efficiency after the same element was heat-treated was calculated.

A relative value was calculated for each of ten manufactured elements of the same kind, and the average relative value thereof was used as a relative value of the elements of that kind.

The heat resistance was evaluated as “A” in a case where the above-described relative value is 0.98 or more, “B” in a case where the above-described relative value is 0.95 or more and less than 0.98, “C” in a case where the above-described relative value is 0.93 or more and less than 0.95, “D” in a case where the above-described relative value is 0.90 or more and less than 0.93, and “E” in a case where the above-described relative value is less than 0.90.

The features of the photoelectric conversion element of each of Examples and Comparative Examples and the results of tests conducted using the photoelectric conversion element of each Examples and Comparative Examples are shown in Table 1 below.

In Tables, the “Kind” column in the “Compound” column indicates the kinds of the compounds used as the compounds for evaluation in the production of the elements.

The “Formula” column indicates whether the specific compound used is a compound represented by any of Formula (1), Formula (1-2), Formula (1-3), Formula (2-2), Formula (3-2), Formula (3-3), Formula (3-4), Formula (3-5), Formula (3-6), Formula (1-4), Formula (1-5), Formula (1-6), Formula (1-7), or Formula (1-8). In a case where a compound for evaluation is a compound that can be represented by a plurality of formulae among these formulae, the compound for evaluation has been represented by the last-described one. For example, in a case where the compound is a compound that can be represented by any of Formula (1), Formula (1-2), and Formula (3-2), the “Formula” column indicates a compound represented by Formula (3-2).

The “U” column indicates the form of a group represented by any of U¹ to U⁶ contained in the specific compound used. The “aromatic ring group” means that U² in Formula (1-2), Formula (1-4), Formula (1-5), Formula (1-6), Formula (1-7), and Formula (1-8) or U³ in Formula (1-3) is an aromatic ring group (this aromatic ring group may have, as a substituent, a group selected from the group consisting of a halogen atom, an alkyl group having a 1 or 2 carbon atoms, which may have a halogen atom, and a cyano group), “4-1F” means a group represented by Formula (4-1) having T¹ that is a fluorine atom, “4-1CN” means a group represented by Formula (4-1) having T¹ that is a cyano group, “4-2” means a group represented by Formula (4-2), “4-3F” means a group represented by Formula (4-3) having T³ that is a fluorine atom, “4-4” means a group represented by Formula (4-4), “4-5F” means a group represented by Formula (4-5) having T⁵ that is a fluorine atom, “4-5CN” means a group represented by Formula (4-5) having T⁵ that is a cyano group, “4-5AL” means a group represented by Formula (4-5) having T⁵ that is an alkyl group, and “4-6” means a group represented by Formula (4-6).

The “Coloring agent” column and the “n-type semiconductor material” column indicate the kind of the coloring agent or the n-type semiconductor material used in the production of the elements, respectively.

The heat resistance test was carried out only in some Examples or Comparative Examples, and in Examples in which the heat resistance test was not carried out, the evaluation column for “Heat resistance” was left blank.

TABLE 1

| | | | | | n-type | Evaluation | | | |
|---|---|---|---|---|---|---|---|---|---|
| | Compound | | | Coloring | semiconductor | | | Variation in | Heat |
| | Kind | Formula | U | agent | material | Sensitivity | Responsiveness | response | resistance |
| Example 1 | G-1 | 2-2 | 4-1F | B-2 | Fullerene C₆₀ | B | A | B | C |
| Example 2 | G-1 | 2-2 | 4-1F | B-3 | Fullerene C₆₀ | B | A | B | |
| Example 3 | G-1 | 2-2 | 4-1F | B-9 | Fullerene C₆₀ | B | A | B | |
| Example 4 | G-2 | 2-2 | 4-1F | B-1 | Fullerene C₆₀ | B | A | B | |
| Example 5 | G-2 | 2-2 | 4-1F | B-3 | Fullerene C₆₀ | B | A | B | |
| Example 6 | G-2 | 2-2 | 4-1F | B-5 | Fullerene C₆₀ | B | A | B | |
| Example 7 | G-2 | 2-2 | 4-1F | B-10 | Fullerene C₆₀ | B | A | B | |
| Example 8 | G-3 | 2-2 | 4-1F | B-3 | Fullerene C₆₀ | B | A | B | |
| Example 9 | G-3 | 2-2 | 4-1F | B-15 | Fullerene C₆₀ | B | A | B | |
| Example 10 | G-3 | 2-2 | 4-1F | B-20 | Fullerene C₆₀ | B | A | B | |
| Example 11 | G-4 | 2-2 | F | B-1 | Fullerene C₆₀ | B | A | B | |
| Example 12 | G-4 | 2-2 | F | B-18 | Fullerene C₆₀ | B | A | B | |
| Example 13 | G-5 | 2-2 | 4-2 | B-2 | Fullerene C₆₀ | A | AA | A | B |
| Example 14 | G-5 | 2-2 | 4-2 | B-4 | Fullerene C₆₀ | A | AA | A | |
| Example 15 | G-6 | 2-2 | 4-1CN | B-1 | Fullerene C₆₀ | A | AA | A | B |
| Example 16 | G-7 | 2-2 | 4-2 | B-1 | Fullerene C₆₀ | A | AA | A | B |
| Example 17 | G-7 | 2-2 | 4-2 | B-2 | Fullerene C₆₀ | A | AA | A | |
| Example 18 | G-7 | 2-2 | 4-2 | B-3 | Fullerene C₆₀ | A | AA | A | |
| Example 19 | G-7 | 2-2 | 4-2 | B-4 | Fullerene C₆₀ | A | AA | A | |
| Example 20 | G-7 | 2-2 | 4-2 | B-5 | Fullerene C₆₀ | A | AA | A | |
| Example 21 | G-7 | 2-2 | 4-2 | B-6 | Fullerene C₆₀ | A | AA | A | |
| Example 22 | G-7 | 2-2 | 4-2 | B-7 | Fullerene C₆₀ | A | AA | A | |
| Example 23 | G-7 | 2-2 | 4-2 | B-8 | Fullerene C₆₀ | A | AA | A | |
| Example 24 | G-7 | 2-2 | 4-2 | B-9 | Fullerene C₆₀ | A | AA | A | |
| Example 25 | G-7 | 2-2 | 4-2 | B-10 | Fullerene C₆₀ | A | AA | A | |
| Example 26 | G-7 | 2-2 | 4-2 | B-11 | Fullerene C₆₀ | A | AA | A | |
| Example 27 | G-7 | 2-2 | 4-2 | B-12 | Fullerene C₆₀ | A | AA | A | |
| Example 28 | G-7 | 2-2 | 4-2 | B-13 | Fullerene C₆₀ | A | AA | A | |
| Example 29 | G-7 | 2-2 | 4-2 | B-14 | Fullerene C₆₀ | A | AA | A | |
| Example 30 | G-7 | 2-2 | 4-2 | B-15 | Fullerene C₆₀ | A | AA | A | |
| Example 31 | G-7 | 2-2 | 4-2 | B-16 | Fullerene C₆₀ | A | AA | A | |
| Example 32 | G-7 | 2-2 | 4-2 | B-17 | Fullerene C₆₀ | A | AA | A | |
| Example 33 | G-7 | 2-2 | 4-2 | B-18 | Fullerene C₆₀ | A | AA | A | |
| Example 34 | G-7 | 2-2 | 4-2 | B-19 | Fullerene C₆₀ | A | AA | A | |
| Example 35 | G-7 | 2-2 | 4-2 | B-20 | Fullerene C₆₀ | A | AA | A | |
| Example 36 | F-2 | 1-2 | Aromatic ring group | B-3 | Fullerene C₆₀ | C | B | C | D |
| Example 37 | F-2 | 1-2 | Aromatic ring group | B-7 | Fullerene C₆₀ | C | B | C | |
| Example 38 | F-2 | 1-2 | Aromatic ring group | B-14 | Fullerene C₆₀ | C | B | C | |
| Example 39 | F-3 | 1-2 | Aromatic ring group | B-3 | Fullerene C₆₀ | C | B | C | |
| Example 40 | F-3 | 1-2 | Aromatic ring group | B-18 | Fullerene C₆₀ | C | B | C | |
| Example 41 | F-3 | 1-2 | Aromatic ring group | B-19 | Fullerene C₆₀ | C | B | C | |
| Example 42 | F-4 | 1-3 | — | B-3 | Fullerene C₆₀ | C | B | C | |
| Example 43 | F-4 | 1-3 | — | B-5 | Fullerene C₆₀ | C | B | C | |
| Example 44 | F-5 | 1-2 | Aromatic ring group | B-3 | Fullerene C₆₀ | C | B | C | |
| Example 45 | F-5 | 1-2 | Aromatic ring group | B-8 | Fullerene C₆₀ | C | B | C | |
| Example 46 | F-6 | 1-2 | Br | B-3 | Fullerene C₆₀ | D | C | D | D |
| Example 47 | F-6 | 1-2 | Br | B-12 | Fullerene C₆₀ | D | C | D | |
| Example 48 | F-7 | 1-2 | Br | B-3 | Fullerene C₆₀ | D | C | D | |
| Example 49 | F-7 | 1-2 | Br | B-17 | Fullerene C₆₀ | D | C | D | |
| Example 50 | F-7 | 1-2 | Br | B-13 | Fullerene C₆₀ | D | C | D | |
| Example 51 | F-1 | 1 | — | B-3 | Fullerene C₆₀ | D | D | D | |
| Example 52 | F-1 | 1 | — | B-1 | Fullerene C₆₀ | D | D | D | |
| Example 53 | I-1-1 | 3-2 | CN | B-3 | Fullerene C₆₀ | A | B | A | |
| Example 54 | I-1-1 | 3-2 | CN | B-1 | Fullerene C₆₀ | A | B | A | |
| Example 55 | I-1-1 | 3-2 | CN | B-6 | Fullerene C₆₀ | A | B | A | |
| Example 56 | I-1-1 | 3-2 | CN | B-9 | Fullerene C₆₀ | A | B | A | |
| Example 57 | I-1-2 | 3-2 | 4-3F | B-3 | Fullerene C₆₀ | A | A | A | |
| Example 58 | I-1-2 | 3-2 | 4-3F | B-8 | Fullerene C₆₀ | A | A | A | |
| Example 59 | I-1-2 | 3-2 | 4-3F | B-13 | Fullerene C₆₀ | A | A | A | |
| Example 60 | I-1-2 | 3-2 | 4-3F | B-15 | Fullerene C₆₀ | A | A | A | |
| Example 61 | I-1-3 | 3-2 | 4-3F | B-3 | Fullerene C₆₀ | A | A | A | |
| Example 62 | I-1-3 | 3-2 | 4-3F | B-2 | Fullerene C₆₀ | A | A | A | |
| Example 63 | I-1-3 | 3-2 | 4-3F | B-10 | Fullerene C₆₀ | A | A | A | |
| Example 64 | I-1-3 | 3-2 | 4-3F | B-19 | Fullerene C₆₀ | A | A | A | |
| Example 65 | I-1-4 | 3-2 | 4-3F | B-3 | Fullerene C₆₀ | A | A | A | |
| Example 66 | I-1-4 | 3-2 | 4-3F | B-5 | Fullerene C₆₀ | A | A | A | |
| Example 67 | I-1-4 | 3-2 | 4-3F | B-11 | Fullerene C₆₀ | A | A | A | |
| Example 68 | I-1-4 | 3-2 | 4-3F | B-12 | Fullerene C₆₀ | A | A | A | |
| Example 69 | I-1-5 | 3-2 | 4-4 | B-3 | Fullerene C₆₀ | A | AA | A | B |
| Example 70 | I-1-5 | 3-2 | 4-4 | B-6 | Fullerene C₆₀ | A | AA | A | |
| Example 71 | I-1-5 | 3-2 | 4-4 | B-12 | Fullerene C₆₀ | A | AA | A | |
| Example 72 | I-1-5 | 3-2 | 4-4 | B-17 | Fullerene C₆₀ | A | AA | A | |
| Example 73 | I-1-5 | 3-2 | 4-4 | B-20 | Fullerene C₆₀ | A | AA | A | |
| Example 74 | I-2-1 | 3-3 | F | B-3 | Fullerene C₆₀ | B | B | B | |
| Example 75 | I-2-1 | 3-3 | F | B-7 | Fullerene C₆₀ | B | B | B | |
| Example 76 | I-2-1 | 3-3 | F | B-13 | Fullerene C₆₀ | B | B | B | |
| Example 77 | I-2-1 | 3-3 | F | B-15 | Fullerene C₆₀ | B | B | B | |
| Example 78 | I-2-2 | 3-3 | CN | B-3 | Fullerene C₆₀ | A | B | A | |
| Example 79 | I-2-3 | 3-3 | 4-5F | B-3 | Fullerene C₆₀ | A | A | A | |
| Example 80 | I-2-3 | 3-3 | 4-5F | B-8 | Fullerene C₆₀ | A | A | A | |
| Example 81 | I-2-3 | 3-3 | 4-5F | B-12 | Fullerene C₆₀ | A | A | A | |
| Example 82 | I-2-3 | 3-3 | 4-5F | B-17 | Fullerene C₆₀ | A | A | A | |
| Example 83 | I-2-4 | 3-3 | 4-5F | B-3 | Fullerene C₆₀ | A | A | A | |
| Example 84 | I-2-5 | 3-3 | 4-5F | B-3 | Fullerene C₆₀ | A | A | A | |
| Example 85 | I-2-5 | 3-3 | 4-5F | B-5 | Fullerene C₆₀ | A | A | A | |
| Example 86 | I-2-5 | 3-3 | 4-5F | B-9 | Fullerene C₆₀ | A | A | A | |
| Example 87 | I-2-6 | 3-3 | 4-5F | B-3 | Fullerene C₆₀ | A | A | A | |
| Example 88 | I-2-7 | 3-3 | 4-5CN | B-1 | Fullerene C₆₀ | A | AA | A | B |
| Example 89 | I-2-7 | 3-3 | 4-5CN | B-2 | Fullerene C₆₀ | A | AA | A | |
| Example 90 | I-2-7 | 3-3 | 4-5CN | B-3 | Fullerene C₆₀ | A | AA | A | |
| Example 91 | I-2-7 | 3-3 | 4-5CN | B-4 | Fullerene C₆₀ | A | AA | A | |
| Example 92 | I-2-7 | 3-3 | 4-5CN | B-5 | Fullerene C₆₀ | A | AA | A | |
| Example 93 | I-2-7 | 3-3 | 4-5CN | B-6 | Fullerene C₆₀ | A | AA | A | |
| Example 94 | I-2-7 | 3-3 | 4-5CN | B-7 | Fullerene C₆₀ | A | AA | A | |
| Example 95 | I-2-7 | 3-3 | 4-5CN | B-8 | Fullerene C₆₀ | A | AA | A | |
| Example 96 | I-2-7 | 3-3 | 4-5CN | B-9 | Fullerene C₆₀ | A | AA | A | |
| Example 97 | I-2-7 | 3-3 | 4-5CN | B-10 | Fullerene C₆₀ | A | AA | A | |
| Example 98 | I-2-7 | 3-3 | 4-5CN | B-11 | Fullerene C₆₀ | A | AA | A | |
| Example 99 | I-2-7 | 3-3 | 4-5CN | B-12 | Fullerene C₆₀ | A | AA | A | |
| Example 100 | I-2-7 | 3-3 | 4-5CN | B-13 | Fullerene C₆₀ | A | AA | A | |
| Example 101 | I-2-7 | 3-3 | 4-5CN | B-14 | Fullerene C₆₀ | A | AA | A | |
| Example 102 | I-2-7 | 3-3 | 4-5CN | B-15 | Fullerene C₆₀ | A | AA | A | |
| Example 103 | I-2-7 | 3-3 | 4-5CN | B-16 | Fullerene C₆₀ | A | AA | A | |
| Example 104 | I-2-7 | 3-3 | 4-5CN | B-17 | Fullerene C₆₀ | A | AA | A | |
| Example 105 | I-2-7 | 3-3 | 4-5CN | B-18 | Fullerene C₆₀ | A | AA | A | |
| Example 106 | I-2-7 | 3-3 | 4-5CN | B-19 | Fullerene C₆₀ | A | AA | A | |
| Example 107 | I-2-7 | 3-3 | 4-5CN | B-20 | Fullerene C₆₀ | A | AA | A | |
| Example 108 | I-2-8 | 3-3 | 4-6 | B-3 | Fullerene C₆₀ | A | AA | A | B |
| Example 109 | I-2-8 | 3-3 | 4-6 | B-4 | Fullerene C₆₀ | A | AA | A | |
| Example 110 | I-2-8 | 3-3 | 4-6 | B-5 | Fullerene C₆₀ | A | AA | A | |
| Example 111 | I-3-1 | 3-4 | — | B-3 | Fullerene C₆₀ | A | A | A | |
| Example 112 | I-4-1 | 3-5 | F | B-3 | Fullerene C₆₀ | C | B | B | |
| Example 113 | I-4-1 | 3-5 | F | B-4 | Fullerene C₆₀ | C | B | B | |
| Example 114 | I-4-1 | 3-5 | F | B-16 | Fullerene C₆₀ | C | B | B | |
| Example 115 | I-4-1 | 3-5 | F | B-17 | Fullerene C₆₀ | C | B | B | |
| Example 116 | I-4-2 | 3-5 | 4-5F | B-3 | Fullerene C₆₀ | B | B | C | |
| Example 117 | I-4-2 | 3-5 | 4-5F | B-18 | Fullerene C₆₀ | B | B | C | |
| Example 118 | I-4-2 | 3-5 | 4-5F | B-19 | Fullerene C₆₀ | B | B | C | |
| Example 119 | I-5-1 | 3-6 | F | B-3 | Fullerene C₆₀ | B | C | B | |
| Example 120 | I-5-1 | 3-6 | F | B-11 | Fullerene C₆₀ | B | C | B | |
| Example 121 | I-5-1 | 3-6 | F | B-12 | Fullerene C₆₀ | B | C | B | |
| Example 122 | I-5-2 | 3-6 | F | B-3 | Fullerene C₆₀ | B | A | B | |
| Example 123 | I-5-2 | 3-6 | F | B-1 | Fullerene C₆₀ | B | A | B | |
| Example 124 | I-5-2 | 3-6 | F | B-17 | Fullerene C₆₀ | B | A | B | |
| Example 125 | I-5-3 | 3-6 | 4-5F | B-3 | Fullerene C₆₀ | B | A | B | |
| Example 126 | I-5-4 | 3-6 | 4-5F | B-3 | Fullerene C₆₀ | B | A | B | |
| Example 127 | I-5-5 | 3-6 | 4-5AL | B-3 | Fullerene C₆₀ | B | B | C | |
| Example 128 | I-5-5 | 3-6 | 4-5AL | B-2 | Fullerene C₆₀ | B | B | C | |
| Example 129 | I-5-5 | 3-6 | 4-5AL | B-9 | Fullerene C₆₀ | B | B | C | |
| Example 130 | I-5-5 | 3-6 | 4-5AL | B-10 | Fullerene C₆₀ | B | B | C | |
| Example 131 | I-5-6 | 3-6 | 4-5CN | B-3 | Fullerene C₆₀ | B | A | B | |
| Example 132 | I-5-6 | 3-6 | 4-5CN | B-12 | Fullerene C₆₀ | B | A | B | |
| Example 133 | I-5-6 | 3-6 | 4-5CN | B-16 | Fullerene C₆₀ | B | A | B | |
| Example 134 | I-5-6 | 3-6 | 4-5CN | B-18 | Fullerene C₆₀ | B | A | B | |
| Example 135 | I-5-7 | 3-6 | 4-6 | B-3 | Fullerene C₆₀ | A | AA | A | B |
| Example 136 | I-5-7 | 3-6 | 4-6 | B-5 | Fullerene C₆₀ | A | AA | A | |
| Example 137 | I-5-7 | 3-6 | 4-6 | B-6 | Fullerene C₆₀ | A | AA | A | |
| Example 138 | H-2 | 1-2 | H | B-3 | Fullerene C₆₀ | C | C | C | |
| Example 139 | H-2 | 1-2 | H | B-20 | Fullerene C₆₀ | C | C | C | |
| Example 140 | H-3 | 1-2 | Aromatic ring group | B-3 | Fullerene C₆₀ | C | C | C | |
| Example 141 | H-4 | 1-2 | Aromatic ring group | B-3 | Fullerene C₆₀ | C | C | C | |
| Example 142 | H-5 | 1-2 | H | B-3 | Fullerene C₆₀ | D | C | C | |
| Example 143 | H-5 | 1-2 | H | B-13 | Fullerene C₆₀ | D | C | C | |
| Example 144 | H-5 | 1-2 | H | B-14 | Fullerene C₆₀ | D | C | C | |
| Example 145 | H-5 | 1-2 | H | B-15 | Fullerene C₆₀ | D | C | C | |
| Example 146 | H-6 | 1-3 | — | B-3 | Fullerene C₆₀ | C | C | C | |
| Example 147 | H-1 | 1 | — | B-3 | Fullerene C₆₀ | D | D | D | |
| Example 148 | H-1 | 1 | — | B-2 | Fullerene C₆₀ | D | D | D | |
| Example 149 | J-1 | 1-4 | F | B-2 | Fullerene C₆₀ | A | AA | A | A |
| Example 150 | J-2 | 1-4 | F | B-10 | Fullerene C₆₀ | A | AA | A | A |
| Example 151 | J-3 | 1-4 | F | B-3 | Fullerene C₆₀ | A | AA | A | A |
| Example 152 | J-4 | 1-4 | F | B-2 | Fullerene C₆₀ | A | AA | A | A |
| Example 153 | J-5 | 1-8 | H | B-3 | Fullerene C₆₀ | A | AA | A | A |
| Example 154 | J-6 | 1-7 | F | B-9 | Fullerene C₆₀ | A | AA | A | A |
| Example 155 | J-7 | 1-8 | H | B-1 | Fullerene C₆₀ | A | AA | A | A |
| Example 156 | J-8 | 1-8 | H | B-3 | Fullerene C₆₀ | A | AA | A | A |
| Example 157 | J-9 | 1-8 | H | B-5 | Fullerene C₆₀ | A | AA | A | A |
| Example 158 | J-10 | 1-8 | H | B-10 | Fullerene C₆₀ | A | AA | A | A |
| Example 159 | J-11 | 1-8 | H | B-3 | Fullerene C₆₀ | A | AA | A | A |
| Example 160 | J-12 | 1-8 | H | B-15 | Fullerene C₆₀ | A | AA | A | A |
| Example 161 | J-13 | 1-7 | F | B-20 | Fullerene C₆₀ | A | AA | A | A |
| Example 162 | J-14 | 1-7 | F | B-1 | Fullerene C₆₀ | A | AA | A | A |
| Example 163 | J-15 | 1-7 | F | B-18 | Fullerene C₆₀ | A | AA | A | A |
| Example 164 | J-16 | 1-8 | H | B-2 | Fullerene C₆₀ | A | AA | A | A |
| Example 165 | J-17 | 1-7 | F | B-4 | Fullerene C₆₀ | A | AA | A | A |
| Example 166 | J-18 | 1-4 | F | B-1 | Fullerene C₆₀ | A | AA | A | A |
| Example 167 | J-19 | 1-6 | F | B-1 | Fullerene C₆₀ | A | AA | A | A |
| Example 168 | J-20 | 1-5 | Aromatic ring group | B-2 | Fullerene C₆₀ | A | AA | A | A |
| Example 169 | J-21 | 1-5 | Aromatic ring group | B-3 | Fullerene C₆₀ | A | AA | A | A |
| Example 170 | J-22 | 1-5 | Aromatic ring group | B-4 | Fullerene C₆₀ | A | AA | A | A |
| Example 171 | J-23 | 1-5 | Aromatic ring group | B-5 | Fullerene C₆₀ | A | AA | A | A |
| Example 172 | K-1 | 1-7 | H | B-2 | Fullerene C₆₀ | A | AA | A | A |
| Example 173 | K-2 | 1-4 | H | B-10 | Fullerene C₆₀ | A | AA | A | A |
| Example 174 | K-3 | 1-8 | H | B-3 | Fullerene C₆₀ | A | AA | A | A |
| Example 175 | K-4 | 1-6 | H | B-2 | Fullerene C₆₀ | A | AA | A | A |
| Example 176 | K-5 | 1-7 | H | B-3 | Fullerene C₆₀ | A | AA | A | A |
| Example 177 | K-6 | 1-4 | H | B-9 | Fullerene C₆₀ | A | AA | A | A |
| Example 178 | K-7 | 1-8 | H | B-1 | Fullerene C₆₀ | A | AA | A | A |
| Example 179 | K-8 | 1-5 | Aromatic ring group | B-3 | Fullerene C₆₀ | A | AA | A | A |
| Example 180 | K-9 | 1-7 | H | B-5 | Fullerene C₆₀ | A | AA | A | A |
| Example 181 | K-10 | 1-4 | H | B-10 | Fullerene C₆₀ | A | AA | A | A |
| Example 182 | K-11 | 1-8 | H | B-3 | Fullerene C₆₀ | A | AA | A | A |
| Example 183 | K-12 | 1-6 | H | B-15 | Fullerene C₆₀ | A | AA | A | A |
| Example 184 | K-13 | 1-5 | H | B-20 | Fullerene C₆₀ | A | AA | A | A |
| Example 185 | K-14 | 1-8 | H | B-1 | Fullerene C₆₀ | A | AA | A | A |
| Example 186 | K-15 | 1-8 | H | B-18 | Fullerene C₆₀ | A | AA | A | A |
| Example 187 | K-16 | 1-8 | H | B-2 | Fullerene C₆₀ | A | AA | A | A |
| Example 188 | K-17 | 1 | — | B-4 | Fullerene C₆₀ | D | D | D | D |
| Example 189 | K-18 | 1 | — | B-1 | Fullerene C₆₀ | D | D | D | D |
| Example 190 | K-1 | 1-7 | H | B-21 | Fullerene C₆₀ | A | AA | A | A |
| Example 191 | K-7 | 1-8 | H | B-22 | Fullerene C₆₀ | A | AA | A | A |
| Example 192 | K-9 | 1-7 | H | B-23 | Fullerene C₆₀ | A | AA | A | A |
| Example 193 | K-11 | 1-8 | H | B-24 | Fullerene C₆₀ | A | AA | A | A |
| Example 194 | K-13 | 1-5 | H | B-25 | Fullerene C₆₀ | A | AA | A | A |
| Example 195 | K-14 | 1-8 | H | B-26 | Fullerene C₆₀ | A | AA | A | A |
| Example 196 | K-19 | 1 | — | B-8 | Fullerene C₆₀ | D | D | D | D |
| Comparative Example 1 | T-1 | — | — | B-3 | Fullerene C₆₀ | F | F | E | E |
| Comparative Example 2 | T-1 | — | — | B-15 | Fullerene C₆₀ | F | E | E | |
| Comparative Example 3 | T-2 | — | — | B-3 | Fullerene C₆₀ | E | F | F | |
| Comparative Example 4 | T-3 | — | — | B-3 | Fullerene C₆₀ | F | F | E | |
| Comparative Example 5 | T-4 | — | — | B-3 | Fullerene C₆₀ | F | E | F | |
| Comparative Example 6 | S-1 | — | — | B-1 | Fullerene C₆₀ | F | E | F | |
| Comparative Example 7 | S-1 | — | — | B-3 | Fullerene C₆₀ | F | E | F | |
| Comparative Example 8 | S-1 | — | — | B-19 | Fullerene C₆₀ | F | E | F | |
| Comparative Example 9 | S-2 | — | — | B-3 | Fullerene C₆₀ | E | F | F | |
| Comparative Example 10 | S-2 | — | — | B-12 | Fullerene C₆₀ | F | E | F | |
| Comparative Example 11 | R-1 | — | — | B-19 | — | E | E | F | |
| Comparative Example 12 | QD | — | — | B-20 | Fullerene C₆₀ | E | E | F | |
| Comparative Example 13 | S-1 | — | — | B-20 | Fullerene C₆₀ | F | E | F | E |
| Comparative Example 14 | T-5 | — | — | B-5 | Fullerene C₆₀ | E | F | F | E |
| Comparative Example 15 | T-6 | — | — | B-6 | Fullerene C₆₀ | E | F | E | E |
| Comparative Example 16 | T-7 | — | — | B-7 | Fullerene C₆₀ | F | E | E | E |
| Comparative Example 17 | T-8 | — | — | B-8 | Fullerene C₆₀ | F | F | E | E |
| Comparative Example 18 | T-9 | — | — | B-9 | Fullerene C₆₀ | F | E | E | E |

From the result illustrated in Table 1, it was confirmed that the photoelectric conversion element according to the embodiment of the present invention using the specific compound for the photoelectric conversion film was excellent to exhibit the effect of the present invention.

On the other hand, in a case where the compounds different from the specific compounds were used, the sensitivity, responsiveness, and preventing properties of variation in response of the obtained photoelectric conversion elements deteriorated as compared to that of the photoelectric conversion element according to the embodiment of the present invention.

In addition, it was confirmed that the effect of the present invention was more excellent in a case where the specific compound was represented by Formula (1-2), Formula (1-3), Formula (2-2), Formula (3-2), Formula (3-3), Formula (3-4), Formula (3-5), Formula (3-6), Formula (1-4), Formula (1-5), Formula (1-6), Formula (1-7), or Formula (1-8).

It was confirmed that the effect of the present invention was still more excellent in a case where the specific compound was represented by Formula (2-2), Formula (3-2), Formula (3-3), Formula (3-4), Formula (3-5), Formula (3-6), Formula (1-4), Formula (1-5), Formula (1-6), Formula (1-7), or Formula (1-8).

From the viewpoint that the effect of the present invention is more excellent, in the compound represented by Formula (1-2), it was confirmed that the group represented by U² was preferably a hydrogen atom or an aromatic ring group. In addition, it was confirmed that R^{Z} is preferably a hydrogen atom (see comparison between results of Examples 36 to 41, 44 to 50, 138 to 145, and the like).

From the viewpoint that the effect of the present invention is more excellent, in the compound represented by Formula (2-2), it was confirmed that the group represented by U⁴ was preferably the group represented by Formula (4-1) having T¹ that is a cyano group, or the group represented by Formula (4-2) (see comparison between results of Examples 1 to 35, and the like).

From the viewpoint that the effect of the present invention is more excellent, in the compound represented by Formula (3-2), the group represented by U⁵ was preferably the group represented by Formula (4-3) or the group represented by Formula (4-4), and it was confirmed that the group represented by Formula (4-4) was more preferable (see comparison between results of Examples 1 to 35, and other examples).

From the viewpoint that the effect of the present invention is more excellent, in the compound represented by Formula (3-3), it was confirmed that the group represented by U⁶ is preferably a cyano group, a group represented by Formula (4-5) having T⁵ that is a fluorine atom, a group represented by Formula (4-5) having T⁵ that is a cyano group, or a group represented by Formula (4-6), more preferably a group represented by Formula (4-5) having T⁵ that is a fluorine atom, a group represented by Formula (4-5) having T⁵ that is a cyano group, or a group represented by Formula (4-6), and still more preferably a group represented by Formula (4-5) having T⁵ that is a cyano group, or a group represented by Formula (4-6) (see comparison between results of Examples 74 to 110).

From the viewpoint that the effect of the present invention is more excellent, in the compound represented by Formula (3-6), the group represented by U⁵ was preferably the group represented by Formula (4-5) having T⁵ that is a fluorine atom, the group represented by Formula (4-5) having T⁵ that is a cyano group, or the group represented by Formula (4-6), and it was confirmed that the group represented by Formula (4-6) was more preferable (see comparison between results of Examples 119 to 137, and the like).

From the viewpoint that the heat resistance of the photoelectric conversion element is more excellent, it was confirmed that the specific compound was preferably a compound represented by Formula (2-2), Formula (3-2), Formula (3-3), Formula (3-6), Formula (1-4), Formula (1-5), Formula (1-6), Formula (1-7), or Formula (1-8), more preferably a group represented by Formula (3-2), Formula (3-3), Formula (3-6), Formula (1-4), Formula (1-5), Formula (1-6), Formula (1-7), or Formula (1-8), and still more preferably a group represented by Formula (1-4), Formula (1-5), Formula (1-6), Formula (1-7), or Formula (1-8).

### EXPLANATION OF REFERENCES

10a, 10b: Photoelectric conversion element
11: Conductive film (lower electrode)
12: Photoelectric conversion film
15: Transparent conductive film (upper electrode)
16A: Electron blocking film
16B: Positive hole blocking film

What is claimed is:

1. A photoelectric conversion element comprising, in the following order:
a conductive film;
a photoelectric conversion film; and
a transparent conductive film,
wherein the photoelectric conversion film contains a compound represented by Formula (1), and a coloring agent, 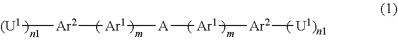
in Formula (1), A is a group represented by any of Formula (2) to Formula (8),
Ar¹ represents an aromatic ring group, where the aromatic ring group represented by Ar¹ may have, as a substituent, a group selected from the group consisting of a halogen atom and an alkyl group having 1 or 2 carbon atoms, which may have a halogen atom,
Ar² represents an aromatic ring group, where the aromatic ring group represented by Ar² may have, as a substituent other than U¹, a group selected from the group consisting of a halogen atom and an alkyl group having 1 or 2 carbon atoms, which may have a halogen atom,
U¹ represents a halogen atom, a cyano group, or an aromatic ring group, where the aromatic ring group represented by U¹ may have, as a substituent, a group selected from the group consisting of a halogen atom, an alkyl group having 1 or 2 carbon atoms, which may have a halogen atom, and a cyano group,
m represents an integer of 0 to 2, and
n1 represents an integer of 0 to 5, 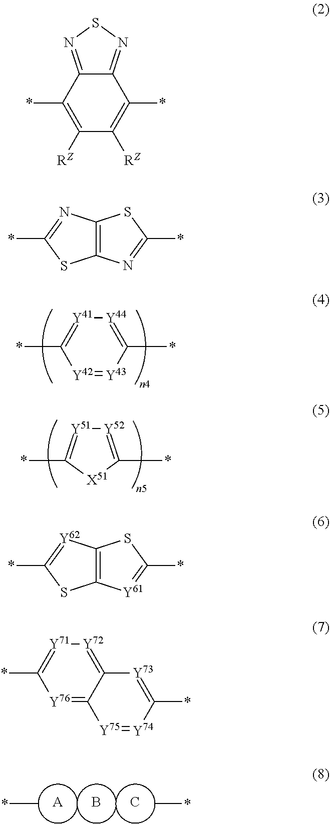
in Formula (2) to Formula (8), * represents a bonding position,
R^{Z} represents a hydrogen atom or a halogen atom,
Y⁴¹ to Y⁴⁴, Y⁵¹, Y⁵², and Y⁷¹ to Y⁷⁶ each independently represent —N═ or —CR═, Y⁶¹ and Y⁶² each independently represent —CR═, R represents a hydrogen atom or a substituent,
X⁵¹ represents a sulfur atom, an oxygen atom, or a selenium atom,
n4 represents 1 or 2,
n5 represents 1 or 2,
A, B, and C each independently represent an aromatic ring group having 5-membered ring or 6-membered ring, A, B, and C are fused with each other to form a fused ring,
in Formula (4), in a case where n4 is 1, at least one of a total of four Y⁴¹ to Y⁴⁴ is —N═ or —CF═, in a case where n4 is 2, at least one of a total of eight Y⁴¹ to Y⁴⁴ is —N═ or —CF═,
in Formula (5), in a case where n5 is 1, at least one of a total of two Y⁵¹ and Y⁵² is —N═ or —CF═, in a case where n5 is 2, at least one of a total of four Y⁵¹ and Y⁵² is —N═ or —CF═,
in Formula (6), at least one of a total of two Y⁶¹ and Y⁶² is —CF═,
in Formula (7), at least one of a total of six Y⁷¹ to Y⁷⁶ is —N═ or —CF═, and
in Formula (8), at least one of ring member atoms constituting the aromatic ring group represented by A, B, and C is —N═ or —CF═.

2. The photoelectric conversion element according to claim 1,
wherein the compound represented by Formula (1) is a compound represented by any of Formula (1-2) to Formula (1-8), 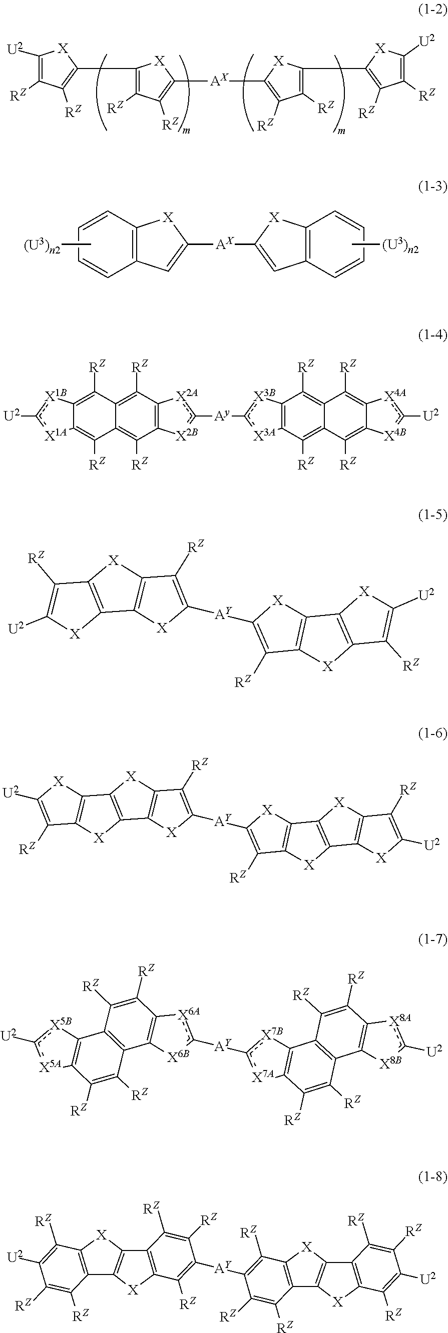
in Formula (1-2) to Formula (1-8), A^{X} is a group represented by Formula (2) or Formula (3),
A^{Y} is a group represented by any of Formula (2) to Formula (8),
U² represents a hydrogen atom, a halogen atom, a cyano group, or an aromatic ring group, where the aromatic ring group represented by U² may have, as a substituent, a group selected from the group consisting of a halogen atom, an alkyl group having 1 or 2 carbon atoms, which may have a halogen atom, and a cyano group,
U³ represents a halogen atom, a cyano group, or an aromatic ring group, where the aromatic ring group represented by U³ may have, as a substituent, a group selected from the group consisting of a halogen atom, an alkyl group having 1 or 2 carbon atoms, which may have a halogen atom, and a cyano group,
R^{Z} represents a hydrogen atom or a halogen atom,
X represents a sulfur atom, an oxygen atom, or a selenium atom,
in Formula (1-4), one of X^{1A} and X^{1B} represents a sulfur atom, an oxygen atom, or a selenium atom, and another of X^{1A} and X^{1B} represents —CR═,
in Formula (1-4), one of X^{2A} and X^{2B} represents a sulfur atom, an oxygen atom, or a selenium atom, and another of X^{2A} and X^{2B} represents —CR═,
in Formula (1-4), one of X^{3A} and X^{3B} represents a sulfur atom, an oxygen atom, or a selenium atom, and another of X^{3A} and X^{3B} represents —CR═,
in Formula (1-4), one of X^{4A} and X^{4B} represents a sulfur atom, an oxygen atom, or a selenium atom, and another of X^{4A} and X^{4B} represents —CR═,
in Formula (1-7), one of X^{5A} and X^{5B} represents a sulfur atom, an oxygen atom, or a selenium atom, and another of X^{5A} and X^{5B} represents —CR═,
in Formula (1-7), one of X^{6A} and X^{6B} represents a sulfur atom, an oxygen atom, or a selenium atom, and another of X^{6A} and X^{6B} represents —CR═,
in Formula (1-7), one of X^{7A} and X^{7B} represents a sulfur atom, an oxygen atom, or a selenium atom, and another of X^{7A} and X^{7B} represents —CR═,
in Formula (1-7), one of X^{8A} and X^{8B} represents a sulfur atom, an oxygen atom, or a selenium atom, and another of X^{8A} and X^{8B} represents —CR═,
R represents a hydrogen atom or a substituent,
m represents an integer of 0 to 2, and
n2 represents 0 or 1.

3. The photoelectric conversion element according to claim 1,
wherein the group represented by Formula (8) is either a group represented by Formula (9) or a group represented by Formula (10), 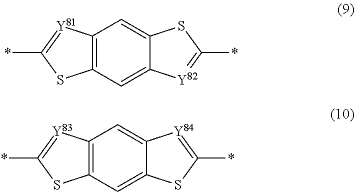
in Formula (9) and Formula (10), * represents a bonding position,
Y⁸¹ to Y⁸⁴ each independently represent —N═ or —CR═, R represents a hydrogen atom or a substituent,
in Formula (9), at least one of a total of two Y⁸¹ and Y⁸² is —N═ or —CF═, and
in Formula (10), at least one of a total of two Y⁸³ and Y⁸⁴ is —N═ or —CF═.

4. The photoelectric conversion element according to claim 1,
wherein the A is a group represented by any of Formula (3) to Formula (8).

5. The photoelectric conversion element according to claim 1,
wherein the compound represented by Formula (1) is a compound represented by any of Formula (1-4) to Formula (1-8).

6. The photoelectric conversion element according to claim 1,
wherein the compound represented by Formula (1) has a molecular weight of 400 to 900.

7. The photoelectric conversion element according to claim 1,
wherein the photoelectric conversion film is a mixture layer formed in a state where the compound represented by Formula (1) and the coloring agent are mixed.

8. The photoelectric conversion element according to claim 1, further comprising one or more interlayers between the conductive film and the transparent conductive film, in addition to the photoelectric conversion film.

9. The photoelectric conversion element according to claim 1,
wherein the photoelectric conversion film further contains a n-type semiconductor material.

10. The photoelectric conversion element according to claim 9,
wherein the n-type semiconductor material includes fullerenes selected from the group consisting of a fullerene and a derivative thereof.

11. An imaging element comprising the photoelectric conversion element according to claim 1.

12. An optical sensor comprising the photoelectric conversion element according to claim 1.

13. A compound represented by Formula (2-2), 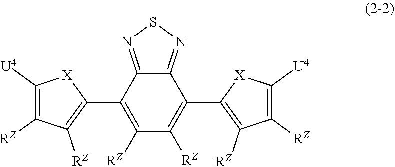
in Formula (2-2), R^{Z} represents a hydrogen atom or a halogen atom,
X represents a sulfur atom, an oxygen atom, or a selenium atom,
U⁴ is a fluorine atom, a group represented by Formula (4-1), or a group represented by Formula (4-2), 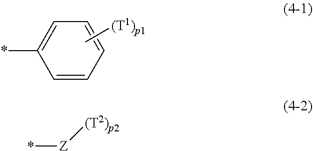
in Formula (4-1) and Formula (4-2), * represents a bonding position,
p1 represents an integer of 1 to 5,
in a case where p1 represents 1, T¹ represents a cyano group,
in a case where p1 represents an integer of 2 to 5, T¹ represents a halogen atom,
Z represents a nitrogen-containing aromatic ring group,
p2 represents an integer of 0 to 4, and
T² represents a halogen atom or a cyano group.

14. A compound represented by any of Formula (3-2) to Formula (3-6), 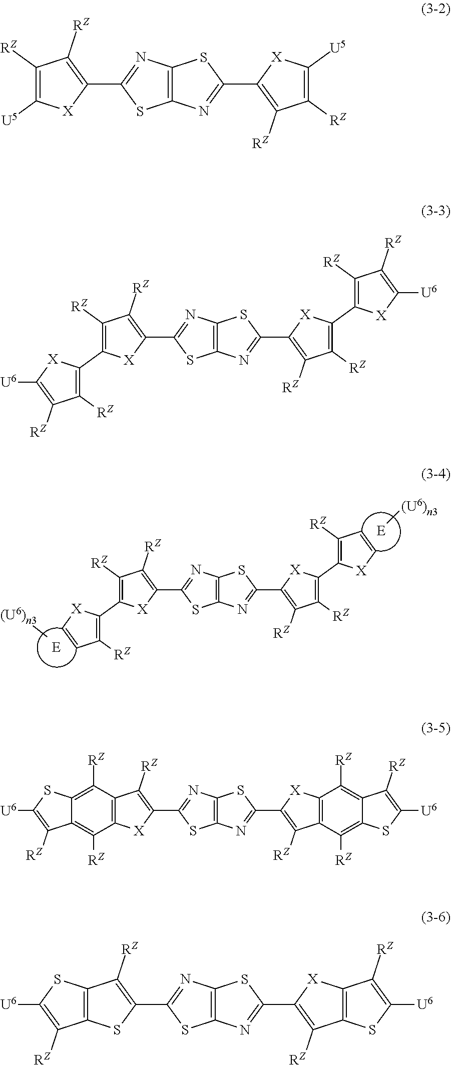
in Formula (3-2) to Formula (3-6), R^{Z} represents a hydrogen atom or a halogen atom,
X represents a sulfur atom, an oxygen atom, or a selenium atom,
E represents an aromatic ring, where the aromatic ring represented by E may have a halogen atom as a substituent other than U⁶,
n3 represents an integer of 0 to 4,
U⁵ is a fluorine atom, a cyano group, a group represented by Formula (4-3), or a group represented by Formula (4-4), and
U⁶ is a fluorine atom, a cyano group, a group represented by Formula (4-5), or a group represented by Formula (4-6), 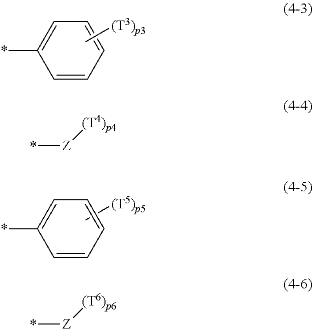
in Formula (4-3) to Formula (4-6), * represents a bonding position,
Z represents a nitrogen-containing aromatic ring group,
p3 represents an integer of 1 to 5,
T³ represents a halogen atom or a cyano group,
p4 represents an integer of 0 to 4,
T⁴ represents a halogen atom, an alkyl group having 1 or 2 carbon atoms, which may have a halogen atom, or a cyano group,
p5 represents an integer of 0 to 5,
T⁵ represents a halogen atom, an alkyl group having 1 or 2 carbon atoms, which may have a halogen atom, or a cyano group,
p6 represents an integer of 0 to 4, and
T⁶ represents a halogen atom, an alkyl group having 1 or 2 carbon atoms, which may have a halogen atom, or a cyano group.

15. A compound represented by Formula (1-4), 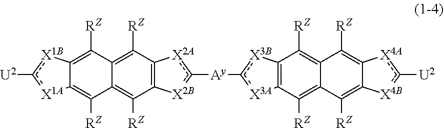
in Formula (1-4), A^{Y} is a group represented by any of Formula (2) to Formula (8),
U² represents a hydrogen atom, a halogen atom, a cyano group, or an aromatic ring group, where the aromatic ring group represented by U² may have, as a substituent, a group selected from the group consisting of a halogen atom, an alkyl group having 1 or 2 carbon atoms, which may have a halogen atom, and a cyano group,
R^{Z} represents a hydrogen atom or a halogen atom, and
in Formula (1-4), one of X^{1A} and X^{1B} represents a sulfur atom, an oxygen atom, or a selenium atom, and another of X^{1A} and X^{1B} represents —CR═,
in Formula (1-4), one of X^{2A} and X^{2B} represents a sulfur atom, an oxygen atom, or a selenium atom, and another of X^{2A} and X^{2B} represents —CR═,
in Formula (1-4), one of X^{3A} and X^{3B} represents a sulfur atom, an oxygen atom, or a selenium atom, and another of X^{3A} and X^{3B} represents —CR═,
in Formula (1-4), one of X^{4A} and X^{4B} represents a sulfur atom, an oxygen atom, or a selenium atom, and another of X^{4A} and X^{4B} represents —CR═, and
R represents a hydrogen atom or a substituent, 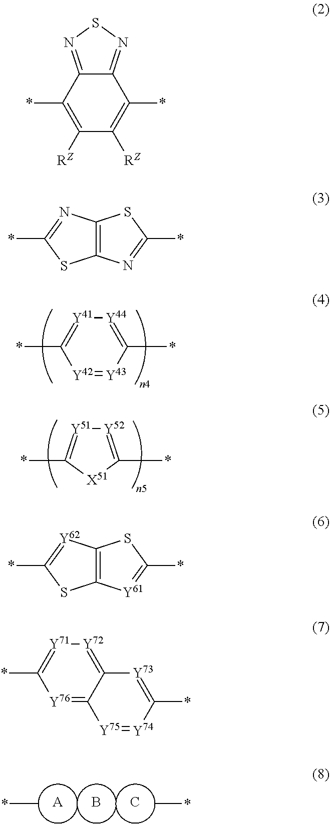
in Formula (2) to Formula (8), * represents a bonding position,
R^{Z} represents a hydrogen atom or a halogen atom,
Y⁴¹ to Y⁴⁴, Y⁵¹, Y⁵², and Y⁷¹ to Y⁷⁶ each independently represent —N═ or —CR═, Y⁶¹ and Y⁶² each independently represent —CR═, R represents a hydrogen atom or a substituent,
X⁵¹ represents a sulfur atom, an oxygen atom, or a selenium atom,
n4 represents 1 or 2,
n5 represents 1 or 2,
A, B, and C each independently represent an aromatic ring group having 5-membered ring or 6-membered ring, A, B, and C are fused with each other to form a fused ring,
in Formula (4), in a case where n4 is 1, at least one of a total of four Y⁴¹ to Y⁴⁴ is —N═ or —CF═, in a case where n4 is 2, at least one of a total of eight Y⁴¹ to Y⁴⁴ is —N═ or —CF═,
in Formula (5), in a case where n5 is 1, at least one of a total of two Y⁵¹ and Y⁵² is —N═ or —CF═, in a case where n5 is 2, at least one of a total of four Y⁵¹ and Y⁵² is —N═ or —CF═,
in Formula (6), at least one of a total of two Y⁶¹ and Y⁶² is —CF═,
in Formula (7), at least one of a total of six Y⁷¹ to Y⁷⁶ is —N═ or —CF═, and
in Formula (8), at least one of ring member atoms constituting the aromatic ring group represented by A, B, and C is —N═ or —CF═.

16. A compound represented by Formula (1-5), 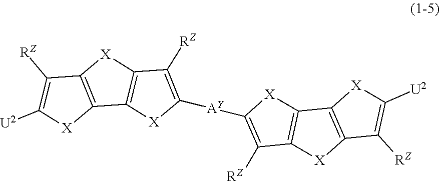
in Formula (1-5), A^{Y} is a group represented by any of Formula (2) to Formula (8),
U² represents a hydrogen atom, a halogen atom, a cyano group, or an aromatic ring group, where the aromatic ring group represented by U² may have, as a substituent, a group selected from the group consisting of a halogen atom, an alkyl group having 1 or 2 carbon atoms, which may have a halogen atom, and a cyano group,
R^{Z} represents a hydrogen atom or a halogen atom, and
X represents a sulfur atom, an oxygen atom, or a selenium atom, 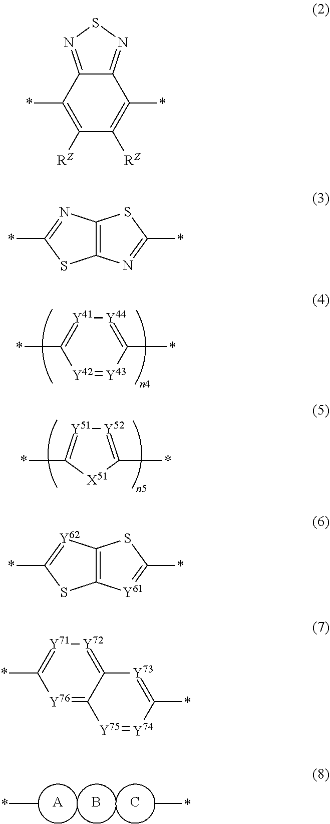
in Formula (2) to Formula (8), * represents a bonding position,
R^{Z} represents a hydrogen atom or a halogen atom,
Y⁴¹ to Y⁴⁴, Y⁵¹, Y⁵², and Y⁷¹ to Y⁷⁶ each independently represent —N═ or —CR═, Y⁶¹ and Y⁶² each independently represent —CR═, R represents a hydrogen atom or a substituent,
X⁵¹ represents a sulfur atom, an oxygen atom, or a selenium atom,
n4 represents 1 or 2,
n5 represents 1 or 2,
A, B, and C each independently represent an aromatic ring group having 5-membered ring or 6-membered ring, A, B, and C are fused with each other to form a fused ring,
in Formula (4), in a case where n4 is 1, at least one of a total of four Y⁴¹ to Y⁴⁴ is —N═ or —CF═, in a case where n4 is 2, at least one of a total of eight Y⁴¹ to Y⁴⁴ is —N═ or —CF═,
in Formula (5), in a case where n5 is 1, at least one of a total of two Y⁵¹ and Y⁵² is —N═ or —CF═, in a case where n5 is 2, at least one of a total of four Y⁵¹ and Y⁵² is —N═ or —CF═,
in Formula (6), at least one of a total of two Y⁶¹ and Y⁶² is —CF═,
in Formula (7), at least one of a total of six Y⁷¹ to Y⁷⁶ is —N═ or —CF═, and
in Formula (8), at least one of ring member atoms constituting the aromatic ring group represented by A, B, and C is —N═ or —CF═.

17. A compound represented by Formula (1-6), 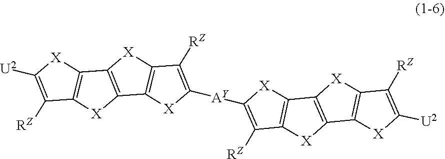
in Formula (1-6), A^{Y} is a group represented by any of Formula (2) to Formula (8),
U² represents a hydrogen atom, a halogen atom, a cyano group, or an aromatic ring group, where the aromatic ring group represented by U² may have, as a substituent, a group selected from the group consisting of a halogen atom, an alkyl group having 1 or 2 carbon atoms, which may have a halogen atom, and a cyano group,
R^{Z} represents a hydrogen atom or a halogen atom, and
X represents a sulfur atom, an oxygen atom, or a selenium atom, 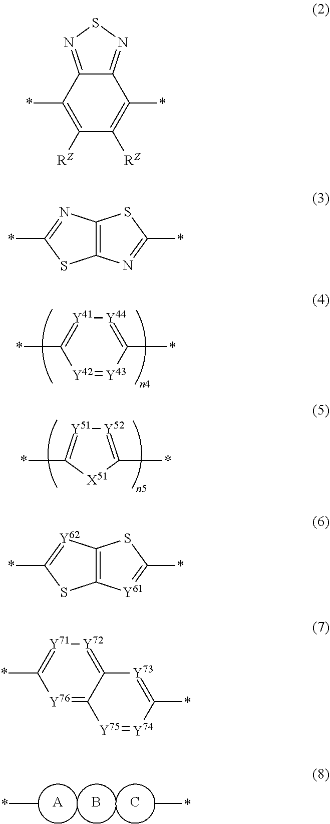
in Formula (2) to Formula (8), * represents a bonding position,
R^{Z} represents a hydrogen atom or a halogen atom,
Y⁴¹ to Y⁴⁴, Y⁵¹, Y⁵², and Y⁷¹ to Y⁷⁶ each independently represent —N═ or —CR═, Y⁶¹ and Y⁶² each independently represent —CR═, R represents a hydrogen atom or a substituent,
X⁵¹ represents a sulfur atom, an oxygen atom, or a selenium atom,
n4 represents 1 or 2,
n5 represents 1 or 2,
A, B, and C each independently represent an aromatic ring group having 5-membered ring or 6-membered ring, A, B, and C are fused with each other to form a fused ring,
in Formula (4), in a case where n4 is 1, at least one of a total of four Y⁴¹ to Y⁴⁴ is —N═ or —CF═, in a case where n4 is 2, at least one of a total of eight Y⁴¹ to Y⁴⁴ is —N═ or —CF═,
in Formula (5), in a case where n5 is 1, at least one of a total of two Y⁵¹ and Y⁵² is —N═ or —CF═, in a case where n5 is 2, at least one of a total of four Y⁵¹ and Y⁵² is —N═ or —CF═,
in Formula (6), at least one of a total of two Y⁶¹ and Y⁶² is —CF═,
in Formula (7), at least one of a total of six Y⁷¹ to Y⁷⁶ is —N═ or —CF═, and
in Formula (8), at least one of ring member atoms constituting the aromatic ring group represented by A, B, and C is —N═ or —CF═.

18. A compound represented by Formula (1-7), 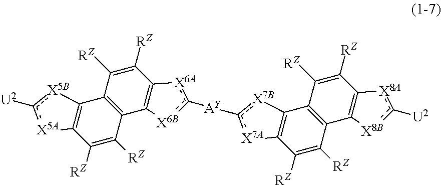
in Formula (1-7), A^{Y} is a group represented by any of Formula (2) to Formula (8),
U² represents a hydrogen atom, a halogen atom, a cyano group, or an aromatic ring group, where the aromatic ring group represented by U² may have, as a substituent, a group selected from the group consisting of a halogen atom, an alkyl group having 1 or 2 carbon atoms, which may have a halogen atom, and a cyano group,
R^{Z} represents a hydrogen atom or a halogen atom,
in Formula (1-7), one of X^{5A} and X^{5B} represents a sulfur atom, an oxygen atom, or a selenium atom, and another of X^{5A} and X^{5B} represents —CR═,
in Formula (1-7), one of X^{6A} and X^{6B} represents a sulfur atom, an oxygen atom, or a selenium atom, and another of X^{6A} and X^{6B} represents —CR═,
in Formula (1-7), one of X^{7A} and X^{7B} represents a sulfur atom, an oxygen atom, or a selenium atom, and another of X^{7A} and X^{7B} represents —CR═,
in Formula (1-7), one of X^{8A} and X^{8B} represents a sulfur atom, an oxygen atom, or a selenium atom, and another of X^{8A} and X^{8B} represents —CR═, and
R represents a hydrogen atom or a substituent, 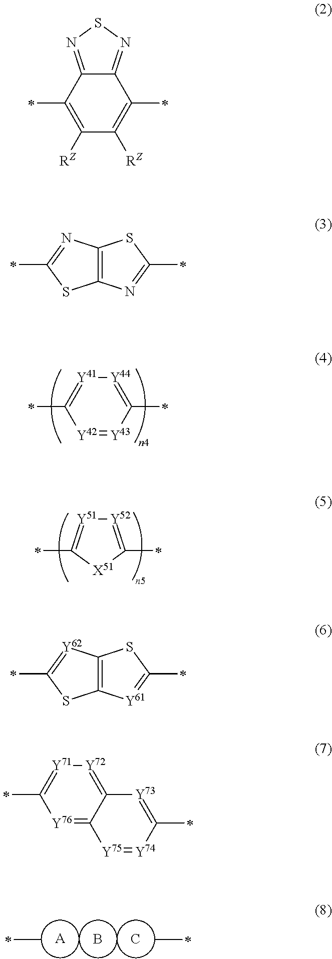
in Formula (2) to Formula (8), * represents a bonding position,
R^{Z} represents a hydrogen atom or a halogen atom,
Y⁴¹ to Y⁴⁴, Y⁵¹, Y⁵², and Y⁷¹ to Y⁷⁶ each independently represent —N═ or —CR═, Y⁶¹ and Y⁶² each independently represent —CR═, R represents a hydrogen atom or a substituent,
X⁵¹ represents a sulfur atom, an oxygen atom, or a selenium atom,
n4 represents 1 or 2,
n5 represents 1 or 2,
A, B, and C each independently represent an aromatic ring group having 5-membered ring or 6-membered ring, A, B, and C are fused with each other to form a fused ring,
in Formula (4), in a case where n4 is 1, at least one of a total of four Y⁴¹ to Y⁴⁴ is —N═ or —CF═, in a case where n4 is 2, at least one of a total of eight Y⁴¹ to Y⁴⁴ is —N═ or —CF═,
in Formula (5), in a case where n5 is 1, at least one of a total of two Y⁵¹ and Y⁵² is —N═ or —CF═, in a case where n5 is 2, at least one of a total of four Y⁵¹ and Y⁵² is —N═ or —CF═,
in Formula (6), at least one of a total of two Y⁶¹ and Y⁶² is —CF═,
in Formula (7), at least one of a total of six Y⁷¹ to Y⁷⁶ is —N═ or —CF═, and
in Formula (8), at least one of ring member atoms constituting the aromatic ring group represented by A, B, and C is —N═ or —CF═.

19. A compound represented by Formula (1-8), 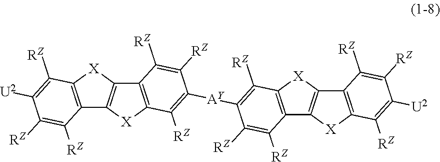
in Formula (1-8), A^{Y} is a group represented by any of Formula (2) to Formula (8),
U² represents a hydrogen atom, a halogen atom, a cyano group, or an aromatic ring group, where the aromatic ring group represented by U² may have, as a substituent, a group selected from the group consisting of a halogen atom, an alkyl group having 1 or 2 carbon atoms, which may have a halogen atom, and a cyano group,
R^{Z} represents a hydrogen atom or a halogen atom, and
X represents a sulfur atom, an oxygen atom, or a selenium atom, 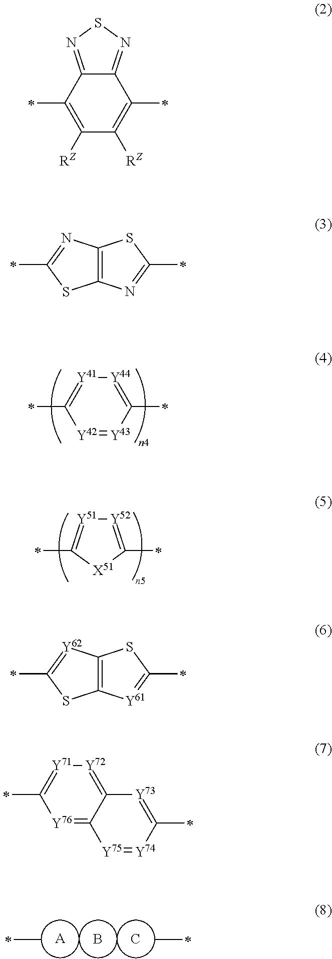
in Formula (2) to Formula (8), * represents a bonding position,
R^{Z} represents a hydrogen atom or a halogen atom,
Y⁴¹ to Y⁴⁴, Y⁵¹, Y⁵², and Y⁷¹ to Y⁷⁶ each independently represent —N═ or —CR═, Y⁶¹ and Y⁶² each independently represent —CR═, R represents a hydrogen atom or a substituent,
X⁵¹ represents a sulfur atom, an oxygen atom, or a selenium atom,
n4 represents 1 or 2,
n5 represents 1 or 2,
A, B, and C each independently represent an aromatic ring group having 5-membered ring or 6-membered ring, A, B, and C are fused with each other to form a fused ring,
in Formula (4), in a case where n4 is 1, at least one of a total of four Y⁴¹ to Y⁴⁴ is —N═ or —CF═, in a case where n4 is 2, at least one of a total of eight Y⁴¹ to Y⁴⁴ is —N═ or —CF═,
in Formula (5), in a case where n5 is 1, at least one of a total of two Y⁵¹ and Y⁵² is —N═ or —CF═, in a case where n5 is 2, at least one of a total of four Y⁵¹ and Y⁵² is —N═ or —CF═,
in Formula (6), at least one of a total of two Y⁶¹ and Y⁶² is —CF═,
in Formula (7), at least one of a total of six Y⁷¹ to Y⁷⁶ is —N═ or —CF═, and
in Formula (8), at least one of ring member atoms constituting the aromatic ring group represented by A, B, and C is —N═ or —CF═.

20. The compound according to claim 13,
wherein the compound has a molecular weight of 400 to 900.
